(19) United States
(12) Patent Application Publication
(10) Pub. No.: US 2024/0390511 A1
(43) Pub. Date: Nov. 28, 2024

(54) METHODS OF TREATING CANCER

(71) Applicant: The Regents of the University of California, Oakland, CA (US)

(72) Inventors: Dennis Slamon, Los Angeles, CA (US); Neil A. O'Brien, Los Angeles, CA (US)

(21) Appl. No.: 18/674,612

(22) Filed: May 24, 2024

Related U.S. Application Data
(60) Provisional application No. 63/468,817, filed on May 25, 2023.

(51) Int. Cl. A61K 47/68; A61P 35/00

(52) U.S. Cl. CPC A61K 47/6849; A61P 35/00

(57) ABSTRACT

Described herein are methods for inhibiting a solid tumor expressing claudin-6 in a human subject, comprising administering to the human subject an effective amount of a composition comprising conjugates of a CLDN6-specific antigen-binding protein covalently bound to heterologous moieties comprising structural formula (I):

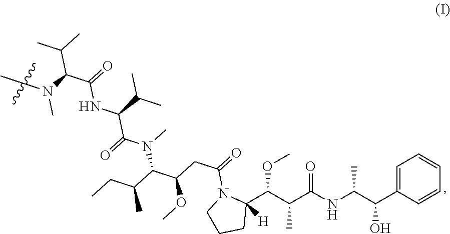 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein
a first plurality of the conjugates are bound to four heterologous moieties comprising structural formula (I);
at least about 95% of the first plurality of conjugates are structurally homogenous; and
the effective amount is in a range of about
1.0 mg of conjugate/kg weight of the subject to 10 mg of conjugate/kg weight of the subject.

### CROSS-REFERENCE TO RELATED APPLICATIONS

This application claims the benefit of U.S. Provisional Application No. 63/468,817, filed on May 25, 2023, the entire contents of which are incorporated herein in their entirety by this reference.

### REFERENCE TO ELECTRONIC SEQUENCE LISTING

The application contains a Sequence Listing which has been submitted electronically in XML format and is hereby incorporated by reference in its entirety. Said XML copy, created on May 23, 2024, is named “UCL-00525.xml” and is 839,728 bytes in size. The sequence listing contained in this XML file is part of the specification and is hereby incorporated by reference herein in its entirety.

### BACKGROUND

### Claudin-6

Claudin-6 (CLDN6) is a member of the claudin family that consists of 27 four-transmembrane domain proteins located at the tight junctions between epithelial cells where they play a critical role in barrier function. CLDN6 expression in non-cancerous tissues is limited to tissues involved in the early stages of development, while it has been reported to be aberrantly expressed in various cancer types including ovarian, gastric, embryonic, pediatric, and endometrial cancer.

CLDN6 is expressed in a significant portion of ovarian, endometrioid, and testicular cancers as well as in a subset of non-small cell lung carcinomas using The Cancer Genome Atlas (TCGA) dataset. Analysis of cancer cell lines revealed CLDN6-positive cell lines within the ovarian, lung, endometrial, and bladder cancer. No notable expression of CLDN6 was detected in normal tissue.

### Ovarian Cancer

Ovarian cancer accounts for more deaths than any other cancer of the female reproductive system. The overall 5-year relative survival rate is 49.1%.

Generally, ovarian cancers also include fallopian tube and peritoneal cancers as they are closely related and treated the same way. The most common type of ovarian cancer is epithelial carcinoma, which accounts for 85% to 90% of ovarian cancers. Rarer forms of ovarian cancer include germ cell malignancies and sex cord stromal tumors.

The majority of women are diagnosed with advanced stages of ovarian cancer, and approximately 80% of these patients will have tumor progression or recurrent disease after initial treatment. Ovarian cancer that recurs within 6 months of platinum-based chemotherapy is categorized as platinum-resistant, while ovarian cancer that recurs more than 6 months after platinum-based chemotherapy is platinum-sensitive. Patients with platinum-resistant ovarian cancer typically undergo additional treatment with single agents, such as pegylated liposomal doxorubicin (PLD), topotecan, gemcitabine, and weekly paclitaxel, but the objective response rates (ORR) for these agents are low (10-15%) and the duration of response (DOR) is short (4 months).

In addition to the single agents, bevacizumab in combination with chemotherapy and poly(adenosine diphosphate [ADP]-ribose polymerase (PARP) inhibitors have been studied in the treatment of advanced ovarian cancer. However, these treatments also present issues. While bevacizumab in combination showed increased progression free survival (PFS) in clinical trials, it is unclear whether this PFS is clinically significant and will lead to improvements in quality of life or overall survival (OS). Bevacizumab has also raised concerns regarding gastrointestinal (GI) toxicity. PARP inhibitors, such as olaparib and niraparib, are administered as maintenance therapy in patients with BRCA mutations, which represents a low percentage of patients (about 17% of patients in high serous subset of ovarian cancer).

Ovarian cancer patients eventually progress on all available treatments and thus, additional therapies to prolong survival are needed. Platinum-resistant advanced ovarian cancer remains an area of unmet need.

### Non-Small Cell Lung Cancer

Lung cancer is the second most common cancer and by far the leading cause of cancer deaths, accounting for almost 25% of cancer deaths. The 5-year relative survival rate is 21.7%. Cigarette smoking is the most important risk factor, accounting for 85% to 90% of lung cancers. However, the risk of developing lung cancer is associated with the extent of smoking and exposure to other carcinogenic factors, such as asbestos.

Lung cancer is made up of two different types: non-small cell lung cancer (NSCLC) and small cell lung cancer. NSCLC makes up the majority of cases in about 85% of patients. The different classifications of NSCLC include adenocarcinoma (approximately 40% of lung cancers), squamous cell carcinoma (25% to 30% of lung cancers), large cell carcinoma (5% to 10% of lung cancers), and NSCLC—not otherwise specified (NOS).

Systemic therapy for advanced NSCLC is selected according to the presence of specific biomarkers. Molecular alterations that predict response to treatment (e.g., epidermal growth factor receptor [EGFR] mutations, anaplastic lymphoma kinase [ALK] rearrangements, ROS1 rearrangements, and BRAF V600E mutations) are present in approximately 30% of patients with NSCLC. Targeted therapy for these alterations improves progression-free survival compared with cytotoxic chemotherapy. Recently inhibitors of KRAS G12C have demonstrated antitumor activity and sotorasib has been approved. For patients without biomarkers indicating susceptibility to specific targeted treatments, immune checkpoint inhibitor-containing regimens either as monotherapy or in combination with chemotherapy are superior to chemotherapy alone.

Despite numerous advances in NSCLC over the past decade with targeted therapies and immunotherapies, resistance is common and the development of new therapeutics is needed.

There exists a need for CLDN6-targeted therapeutics for treating cancer, such as ovarian cancer and NSCLC, in humans.

### SUMMARY

In certain embodiments, the disclosure relates to a method for inhibiting a solid tumor expressing claudin-6 in a human subject, comprising administering to the human subject an effective amount of a composition comprising conjugates of a CLDN6-specific antigen-binding protein covalently bound to heterologous moieties comprising structural formula (I):

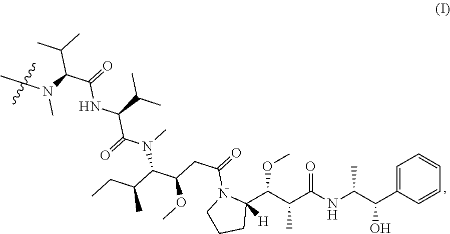 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein
a first plurality of the conjugates are bound to about four heterologous moieties comprising structural formula (I);
at least about 95% of the first plurality of conjugates are structurally homogenous; and
the effective amount is in a range of about
   1.7 mg/kg to 6 mg/kg;
   2.0 mg/kg to 6 mg/kg; or
   2.4 mg/kg to 6 mg/kg.

In certain embodiments, the disclosure relates to any of the methods disclosed herein, wherein the CLDN6-specific antigen-binding protein comprises an antibody that binds CLDN6 or antigen-binding fragment thereof comprising:
(i) HC CDR1 having a sequence GFTFSNYW (SEQ ID NO: 23);
(ii) HC CDR2 having a sequence IRLKSDNYAT (SEQ ID NO: 24),
(iii) HC CDR3 having a sequence XDGPPSGX (SEQ ID NO: 457), wherein X at position 1 is N and X at position 8 is S, T, A, C, or Y,
(iv) LC CDR1 having a sequence ENIYSY (SEQ ID NO: 20),
(v) LC CDR2 having a sequence NAK (SEQ ID NO: 21), and
(vi) LC CDR3 having a sequence QHHYTVPWT (SEQ ID NO: 22).

### BRIEF DESCRIPTION OF THE FIGURES

FIG. 1A is a bar graph showing tumor response (max change in tumor size by RECIST 1.1) at dose levels 0.2 to 2.4 mg/kg of TORL-1-23 in humans and FIG. 1B shows dose levels 0.2 to 3.0 mg/kg of TORL-1-23 in humans.

FIG. 2A is a graph showing change in tumor size over time at dose levels 0.2 to 2.4 mg/kg of TORL-1-23 in humans and FIG. 2B shows tumor size over time at dose levels 0.2 to 3.0 mg/kg of TORL-1-23 in humans.

FIG. 3 is a collection of line graphs assessing anti-tumor activity of TORL-1-23 administered intravenously once weekly at either 2.5 or 5.0 mg/kg in human cancer murine xenograft models with CLDN6-positive ovarian (OV90), bladder (UMUC4) or endometrial (ARK2) cancer or CLDN6-negative melanoma (M202) cancer.

FIGS. 4A and 4A continued are a collection of line graphs showing mean plasma concentrations of TORL-1-23 total antibody (conjugated and unconjugated to four MMAEs), TORL-1-23 (comprising 4 MMAEs), and unconjugated (or free) MMAE following TORL-1-23 administration every 3 weeks at doses of 0.2, 0.4, 0.8, 1.0, 1.3, 1.7, 2.0, and 2.4 mg/kg for cycle 1. FIGS. 4B and 4B continued are a collection of line graphs showing mean plasma concentrations of TORL-1-23 total antibody (conjugated and unconjugated to four MMAEs), TORL-1-23 (comprising 4 MMAEs), and unconjugated (or free) MMAE following TORL-1-23 administration every 3 weeks at doses of 0.2, 0.4, 0.8, 1.0, 1.3, 1.7, 2.0, 2.4, and 3.0 mg/kg for cycles 1 and 3.

FIG. 5 is a line graph showing dose normalized unconjugated MMAE concentrations after a single administration of 0.2, 0.4, 0.8, 1.0, 1.3, 1.7, 2.0, and 2.4 mg/kg of TORL-1-23 (filled circles) or after a single administration of 2.4 mg/kg dose of eight other leading ADCs, ADC1-ADC8, which have undergone phase 1 clinical trial (filled symbols).

### DETAILED DESCRIPTION

### Overview

Described herein are methods for inhibiting a solid tumor expressing claudin-6 in a human subject, comprising administering to the human subject an effective amount of a composition comprising conjugates of a CLDN6-specific antigen-binding protein covalently bound to heterologous moieties comprising structural formula (I):

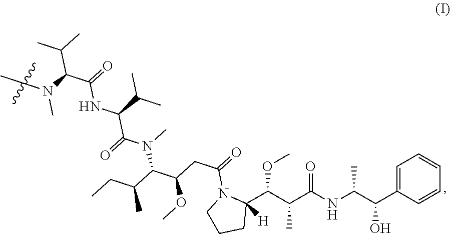 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein
a first plurality of the conjugates are bound to about four heterologous moieties comprising structural formula (I);
at least 95% of the first plurality of conjugates are structurally homogenous; and
the effective amount is in the range of about 1.0 mg conjugate/kg weight of the subject to about 10 mg conjugate/kg weight of the subject.

As used herein, the term “about” when used before a numerical designation, e.g., temperature, time, amount, concentration, and such other, including a range, indicates approximations which may vary by plus or minus (+/−) 5%, 4%, 3%, 2% or 1%.

In certain embodiments, the solid tumor is ovarian cancer, primary peritoneal cancer, fallopian tube cancer, non-small cell lung cancer, or testicular cancer.

In certain embodiments, after administration, the human subject does not experience peripheral neuropathy of grade 3 or higher severity, does not experience alopecia of grade 3 or higher severity, does not experience fatigue of grade 3 or higher severity, does not experience nausea, vomiting or anorexia of grade 3 or higher severity, or does not experience constipation of grade 3 or higher severity, or the human subject does not experience any combination of two or more of these adverse events. In certain embodiments, after administration, the human subject does not experience peripheral neuropathy of grade 3 or higher severity. In certain embodiments, after administration, the human subject does not experience alopecia of grade 3 or higher severity. In certain embodiments, after administration, the human subject does not experience fatigue of grade 3 or higher severity. In certain embodiments, after administration, the human subject does not experience nausea, vomiting or anorexia of grade 3 or higher severity. In certain embodiments, after administration, the human subject does not experience constipation of grade 3 or higher severity. For purposes of this disclosure, widely accepted criteria for documentation and classification of adverse events will be utilized (i.e., National Cancer Institute (NCI) Common Terminology Criteria for Adverse Events (CTCAE) Version 5.0). For example, a “grade 3” adverse event may be a severe or medically significant adverse event that is not immediately life-threatening, but which requires hospitalization. Grade 3 adverse events may limit a patient's ability to bathe, dress or undress, feed himself or herself, use the toilet, or take medication.

In certain embodiments, after administration, treatment-related grade 3 or higher severity peripheral neuropathy occurs in less than 30% of patients treated, treatment-related grade 3 or higher severity alopecia occurs in less than 30% of patients treated, or treatment-related grade 3 or higher severity nausea, vomiting or anorexia occurs in less than 30% of patients treated, or any combination of two or more of these treatment-related adverse events occurs in less than 30% of patients treated. Preferably, after administration, treatment-related grade 3 or higher severity peripheral neuropathy occurs in less than 20% of patients treated, treatment-related grade 3 or higher severity alopecia occurs in less than 20% of patients treated, or treatment-related grade 3 or higher severity nausea, vomiting or anorexia occurs in less than 20% of patients treated, or any combination of two or more of these treatment-related adverse events occurs in less than 20% of patients treated. More preferably, after administration, treatment-related grade 3 or higher severity peripheral neuropathy occurs in less than 10% of patients treated, treatment-related grade 3 or higher severity alopecia occurs in less than 10% of patients treated, or treatment-related grade 3 or higher severity nausea, vomiting or anorexia occurs in less than 10% of patients treated, or any combination of two or more of these treatment-related adverse events occurs in less than 10% of patients treated. In certain embodiments, after administration, no human subject experiences an adverse event of grade 3 or higher severity.

In certain embodiments, after administration, the human subject does not experience any adverse event of grade 3 or higher severity.

In certain embodiments, after administration, the human subject does not experience peripheral neuropathy of grade 2 or higher severity, does not experience alopecia of grade 2 or higher severity, does not experience fatigue of grade 2 or higher severity, does not experience nausea, vomiting or anorexia of grade 2 or higher severity, or does not experience constipation of grade 2 or higher severity, or the human subject does not experience any combination of two or more of these adverse events. In certain embodiments, after administration, the human subject does not experience peripheral neuropathy of grade 2 or higher severity. In certain embodiments, after administration, the human subject does not experience alopecia of grade 2 or higher severity. In certain embodiments, after administration, the human subject does not experience fatigue of grade 2 or higher severity. In certain embodiments, after administration, the human subject does not experience nausea, vomiting or anorexia of grade 2 or higher severity. In certain embodiments, after administration, the human subject does not experience constipation of grade 2 or higher severity. For purposes of this disclosure, widely accepted criteria for documentation and classification of adverse events will be utilized (i.e., National Cancer Institute (NCI) Common Terminology Criteria for Adverse Events (CTCAE) Version 5.0). For example, a “grade 2” adverse event is an adverse event of moderate severity, which limits age-appropriate instrumental activities of daily life, such as preparing meals, shopping for groceries or clothes, using the telephone, or managing money. Minimal, local or noninvasive intervention may be indicated for a subject experiencing a grade 2 adverse event.

In certain embodiments, after administration, treatment-related grade 2 or higher severity peripheral neuropathy occurs in less than 40% of patients treated, treatment-related grade 2 or higher severity alopecia occurs in less than 40% of patients treated, or treatment-related grade 2 or higher severity nausea, vomiting or anorexia occurs in less than 40% of patients treated, or any combination of two or more of these treatment-related adverse events occurs in less than 40% of patients treated. In some embodiments, after administration, treatment-related grade 2 or higher severity peripheral neuropathy occurs in less than 30% of patients treated, treatment-related grade 2 or higher severity alopecia occurs in less than 30% of patients treated, or treatment-related grade 2 or higher severity nausea, vomiting or anorexia occurs in less than 30% of patients treated, or any combination of two or more of these treatment-related adverse events occurs in less than 30% of patients treated. Preferably, after administration, treatment-related grade 2 or higher severity peripheral neuropathy occurs in less than 20% of patients treated, treatment-related grade 2 or higher severity alopecia occurs in less than 20% of patients treated, or treatment-related grade 2 or higher severity nausea, vomiting or anorexia occurs in less than 20% of patients treated, or any combination of two or more of these treatment-related adverse events occurs in less than 20% of patients treated. More preferably, after administration, treatment-related grade 2 or higher severity peripheral neuropathy occurs in less than 10% of patients treated, treatment-related grade 2 or higher severity alopecia occurs in less than 10% of patients treated, or treatment-related grade 2 or higher severity nausea, vomiting or anorexia occurs in less than 10% of patients treated, or any combination of two or more of these treatment-related adverse events occurs in less than 10% of patients treated.

In other embodiments, after administration, the human subject does not experience any adverse event of grade 2 or higher severity.

### Use

The conjugates of the present disclosure are useful for inhibiting tumor growth. Without being bound to a particular theory, the inhibiting action of the conjugates provided herein allow such entities to be useful in methods of treating cancer.

In some embodiments, the method provides that prior to administration of CLDN6 conjugates of the present disclosure to the human subject, a hematopoietic protein such as a colony stimulating factor (CSF) such as granulocyte-colony stimulating factor (G-CSF) or granulocyte macrophage-CSF (GM-CSF) is administered to the subject. In some embodiments, G-CSF pre-treatment is preferred.

Accordingly, provided herein are methods of inhibiting tumor growth in a subject and methods of reducing tumor size in a subject. In various embodiments, the methods comprise administering to the subject the pharmaceutical composition of the present disclosure in an amount effective for inhibiting tumor growth or reducing tumor size in the subject. In various aspects, the growth of an ovarian tumor, melanoma tumor, bladder tumor, or endometrial tumor is inhibited. In various aspects, the size of an ovarian tumor, melanoma tumor, bladder tumor, or endometrial tumor is reduced.

As used herein, the term “inhibit” or “reduce” and words stemming therefrom may not be a 100% or complete inhibition or reduction. Rather, there are varying degrees of inhibition or reduction of which one of ordinary skill in the art recognizes as having a potential benefit or therapeutic effect. In this respect, the antigen-binding proteins of the present disclosure may inhibit tumor growth or reduce tumor size to any amount or level. In various embodiments, the inhibition provided by the methods of the present disclosure is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition). In various embodiments, the reduction provided by the methods of the present disclosure is at least or about a 10% reduction (e.g., at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, at least or about a 95% reduction, at least or about a 98% reduction).

Additionally provided herein are methods of treating a subject with cancer, e.g., CLDN6-expressing cancer. In various embodiments, the method comprises administering to the subject the pharmaceutical composition of the present disclosure in an amount effective for treating the cancer in the subject.

In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In various aspects, the cancer is ovarian cancer, melanoma, bladder cancer, lung cancer, liver cancer, endometrial cancer. In various aspects, the cancer is any cancer characterized by moderate to high expression of CLDN6.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In various aspects, the methods treat by way of delaying the onset or recurrence of the cancer by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject.

### Dosages

The active agents of the disclosure are believed to be useful in methods of inhibiting tumor growth, as well as other methods, as further described herein, including methods of treating cancer. For purposes of the disclosure, the amount or dose of the active agent administered should be sufficient to effect, e.g., a therapeutic response, in the human subject over a reasonable time frame.

For example, the dose of the active agent of the present disclosure can be about 1.0 to about 10 mg/kg body weight of the subject being treated, from about 1.0 to about 6.0 mg/kg, from about 1.0 to about 5.0 mg/kg, from about 1.0 to about 4.0 mg/kg, or from about 1.0 to about 3.0 mg/kg.

Additionally, in another example, the dose can be about 1.7 to about 6.0 mg/kg body weight of the subject, from about 1.7 to about 5.0 mg/kg, from about 1.7 to about 4.0 mg/kg, from about 1.7 to about 3.0 mg/kg, or from about 1.7 to about 2.0 mg/kg.

In certain examples, the dose can be about 1.7 to about 5.0 mg/kg body weight of the subject, from about 1.7 to about 4.0 mg/kg, or from about 1.7 to about 3.0 mg/kg.

In yet another example, the dose can be about 2.0 to about 10.0 mg/kg body weight of the subject, from about 2.0 to about 6.0 mg/kg, from about 2.0 to about 5.0 mg/kg, from about 2.0 to about 4.0 mg/kg, or from about 2.0 to about 3.0 mg/kg.

In some examples, example, the dose can be about 2.0 to about 6.0 mg/kg body weight of the subject, from about 2.0 to about 5.0 mg/kg, from about 2.0 to about 4.0 mg/kg, or from about 2.0 to about 3.0 mg/kg.

In certain examples, example, the dose can be about 2.0 to about 5.0 mg/kg body weight of the subject, from about 2.0 to about 4.0 mg/kg, or from about 2.0 to about 3.0 mg/kg.

In further examples, the dose can be about 2.4 to about 10.0 mg/kg body weight of the subject, from about 2.4 to about 6.0 mg/kg, from about 2.4 to about 5.0 mg/kg, from about 2.4 to about 4.0 mg/kg, or from about 2.4 to about 3.0 mg/kg.

In some examples, the dose can be about 2.4 to about 6.0 mg/kg body weight of the subject, from about 2.4 to about 5.0 mg/kg, from about 2.4 to about 4.0 mg/kg, or from about 2.4 to about 3.0 mg/kg.

In certain examples, the dose can be about 2.4 to about 5.0 mg/kg body weight of the subject, from about 2.4 to about 4.0 mg/kg, or from about 2.4 to about 3.0 mg/kg.

In further examples, the dose can be about 3.0 to about 5.0 mg/kg body weight of the subject, from about 3.0 to about 4.0 mg/kg, or from about 3.0 to about 3.6 mg/kg.

### Conjugates and Conjugation

The present disclosure relates to antigen-binding proteins attached, linked or conjugated to a second moiety (e.g., a heterologous moiety) comprising structural formula (I):

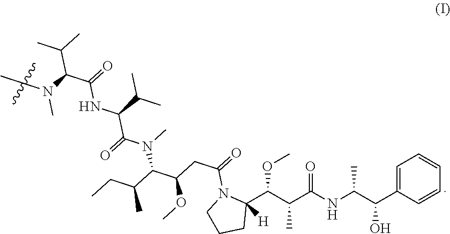 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Accordingly, the present disclosure provides a conjugate comprising an antigen-binding protein and a heterologous moiety and compositions thereof.

The heterologous moiety comprising structural formula (I) derives from monomethyl auristatin E (MMAE), which has the following structural formula:

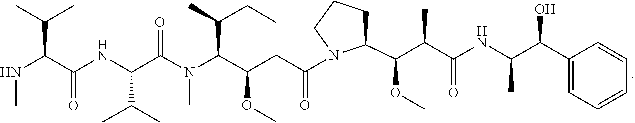 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

IUPAC name: (S)-N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide MMAE, or desmethyl-auristatin E, is a synthetic antineoplastic agent. It is a potent antimitotic drug that inhibits cell division by blocking the polymerization of tubulin.

In certain embodiments, the heterologous moiety (e.g., MMAE) is conjugated to an antigen-binding protein via a linker. In some such embodiments, the linker comprises a peptide having Valine-Citrulline (Val-Cit). In some embodiments, the linker comprises Valine-citrulline coupled with a self-immolative ρ-aminobenzyl (PAB) group, collectively referred to as Val-Cit-PAB. Accordingly, in some embodiments, a conjugate of the present disclosure comprises an antigen-binding protein, Val-Cit-PAB, and MMAE. In preferred embodiments, an antigen-binding protein is conjugated to at least one MMAE, wherein each MMAE is conjugated to the antigen-binding protein via Val-Cit-PAB.

In certain embodiments, the heterologous moiety has structural formula (II):

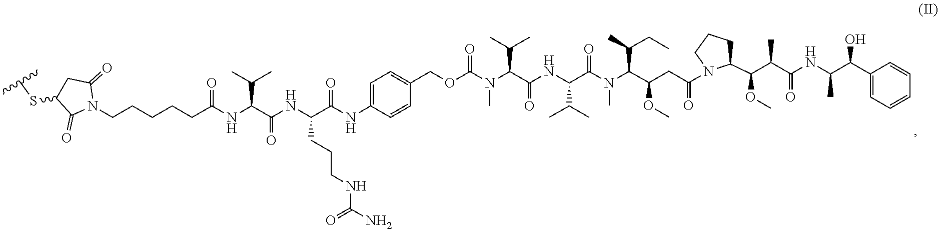 [SMILES: *SC1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C1=O]

wherein

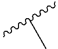

is a covalent thiol bond to the antigen-binding protein. The heterologous moiety having structural formula (II) is called vedotin using International Nonproprietary Names (INN) nomenclature.

The heterologous moiety-to-antigen-binding protein ratio (HAR) represents the number of a heterologous moiety linked per antigen-binding molecule. In some embodiments, the HAR ranges from about 1 to about 15, about 1 to about 10, about 1 to about 9, about 1 to about 8, about 1 to about 7, about 1 to about 6, about 1 to about 5, about 1 to about 4, about 1 to about 3, or about 1 to about 2. In some embodiments, the HAR ranges from about 2 to about 10, about 2 to about 9, about 2 to about 8, about 2 to about 7, about 2 to about 6, about 2 to about 5, about 2 to about 4 or about 2 to about 3. In other embodiments, the HAR is about 2, about 2.5, about 3, about 4, about 5, or about 6. In some embodiments, the HAR ranges from about 2 to about 4. The HAR may be characterized by conventional means such as mass spectrometry, UV/Vis spectroscopy, ELISA assay, and/or HPLC. In some embodiments, the HAR is about 3.0, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, or about 5.0.

In some embodiments, the invention provides a composition comprising structurally “homogeneous” conjugates, wherein the substantial percentage of the antigen-binding proteins are conjugated to a defined number of heterologous moieties at the same specific sites of the antigen-binding protein. In some embodiments, the structurally homogeneous conjugates comprise the HAR of about 1, about 2, about 3, about 4, about 5, about 6, about 7, about 8, about 9, or about 10. In some embodiments, the structurally homogeneous conjugates comprise the HAR of about 2, about 4, about 6, or about 8. In preferred embodiments, the structurally homogeneous conjugates comprise the HAR of about 4. In other preferred embodiments, the structurally homogeneous conjugates comprise the HAR of about 2. In some embodiments, the structurally homogeneous conjugates comprise the HAR of about 3.0, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, or about 5.0. In some embodiments, the structurally homogeneous conjugates comprise greater than or equal to about 85, 86, 87, 88, 89, 90, 91, 92, 93, 94, 95, 96, 97, 98, 99, or 100 percent conjugates with the defined HAR. In some embodiments, the structurally homogeneous conjugates comprise about 85, 86, 87, 88, 89, 90, 91, 92, 93, 94, 95, 96, 97, 98, 99, or 100 percent conjugates with the defined HAR. In some embodiments, the structurally homogeneous conjugates comprise at least about 85, 86, 87, 88, 89, 90, 91, 92, 93, 94, 95, 96, 97, 98, 99, or 100 percent conjugates with the defined HAR. In some embodiments, the homogeneity of the structurally homogeneous conjugates is determined by a chromatogram, e.g., HPLC or any suitable chromatography. In some embodiments, the chromatogram is a HIC chromatogram. In some embodiments, homogeneity of the homogeneous conjugates is determined, or further determined, by protease or tryptic digestion followed by liquid chromatography and tandem mass spectroscopy (e.g., nanoLC-MS/MS) analysis (Farràs et al. (2020) MAbs:12(1):1702262). The structurally homogeneous conjugate may be generated by a site-specific conjugation.

In some embodiments, the heterologous moiety is conjugated to the antigen-binding protein (e.g., antibody) in a site-specific manner. Various site-specific conjugation methods are known in the art, e.g., thiomab or TDC or conjugation at an unpaired cysteine residue or conjugation after reduction of interchain sulfide bonds (WO2020/164561; Junutula et al. (2008) Nat. Biotechnol. 26:925-932; Dimasi et al. (2017) Mol. Pharm. 14:1501-1516; Shen et al. (2012) Nat. Biotechnol. 30:184-9); thiol bridge linker (Behrens et al. (2015) Mol. Pharm. 12:3986-98); conjugation at glutamine using a transglutaminase (Dennler et al. (2013) Methods Mol. Bio. 1045:205-15; Dennler et al. (2014) Bioconjug Chem. 25:569-78); conjugation at engineered unnatural amino acid residues (Axup et al. (2012) Proc Natl Acad Sci U.S.A. 104-16101-6; Tian et al. (2014) Proc Natl Acad Sci U.S.A. 111:1766-71; VanBrunt et al. (2015) Bioconjug Chem 26:2249-60; Zimmerman et al. (2014) Bioconjug Chem 25:351-61); selenocysteine conjugation (Li et al. (2017) Cell Chem Biol 24:433-442); glycan-mediated conjugation (Okeley et al. (2013) Bioconjug Chem 24:1650-5); conjugation at galactose or GalNAc analogues (Ramakrishnan and Qasba (2002) J Biol Chem 277:20833-9; van Geel et al. (2015) Bioconjug Chem 26:2233-42); via glycan engineering (Zhou et al. (2014) Bioconjug Chem 25:510-20; Tang et al. (2017) Nat Protoc 12:1702-1721); via a short peptide tag, such as engineering a glutamine tag or sortase A-mediated transpeptidation (Strop et al. (2013) Chem Biol 20:161-7; Beerli et al. (2015) PLoS One 10:e0131177); and via an aldehyde tag (Wu et al. (2009) Proc Natl Acad Sci U.S.A. 106:3000-5).

### Antigen-Binding Proteins

Provided herein are methods of using antigen-binding proteins, such as antibodies, that bind to Claudin-6 (CLDN6) in ADCs for use in treating cancer.

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in various embodiments, the antibody is an antibody of isotype IgA, IgD, IgE, IgG, or IgM, including any one of IgG1, IgG2, IgG3 or IgG4. In various aspects, the antibody comprises a constant region comprising one or more amino acid modifications, relative to the naturally-occurring counterpart, in order to improve half-life/stability or to render the antibody more suitable for expression/manufacturability. In various instances, the antibody comprises a constant region wherein the C-terminal Lys residue that is present in the naturally-occurring counterpart is removed or clipped.

The antibody can be a monoclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence. Information, including sequence information for human antibody heavy and light chain constant regions is publicly available through the Uniprot database as well as other databases well-known to those in the field of antibody engineering and production. For example, the IgG2 constant region is available from the Uniprot database as Uniprot number P01859, incorporated herein by reference.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment.

In various embodiments, an anti-CLDN6 antibody is selected from the group consisting of a human antibody, a humanized antibody, a chimeric antibody, a monoclonal antibody, a recombinant antibody, an IgG1 antibody, an IgG2 antibody, an IgG3 antibody, and an IgG4 antibody.

### CLDN6 and Epitopes

In various aspects, the CLDN6 is a human CLDN6 having the amino acid sequence of:

(SEQ ID NO: 202)
MASAGMQILGVVLTLLGWVNGLVSCALPMWKVTAFIGNSIVVAQVVWEG
LWMSCVVQSTGQMQCKVYDSLLALPQDLQAARALCVIALLVALFGLLVY
LAGAKCTTCVEEKDSKARLVLTSGIVFVISGVLTLIPVCWTAHAXIRDF
YNPLVAEAQKRELGASLYLGWAASGLLLLGGGLLCCTCPSGGSQGPSHY
MARYSTSAPAISRGPSEYPTKNYV, wherein X is Ile or Val

In various aspects, the human CLDN6 comprises the amino acid sequence of any one of SEQ ID NOs: 1, 178, and 200-202.

In various aspects, the antigen-binding proteins of the present disclosure bind to an epitope within an amino acid sequence of CLDN6. In various aspects, CLDN6 is a human CLDN6 and the antigen-binding proteins of the present disclosure bind to an epitope within an amino acid sequence of human CLDN6, e.g., SEQ ID NOs: 1, 178, and 200-202. By “epitope” is meant the region of or within CLDN6 which is bound by the antigen-binding protein. In some embodiments, the epitope is a linear epitope. “Linear epitope” refers to the region of or within the CLDN6 which is bound by the antigen-binding protein and which region is composed of contiguous amino acids of the amino acid sequence of the CLDN6. The amino acids of a linear epitope are adjacent to each other in the primary structure of the CLDN6. Accordingly, a linear epitope is a fragment or portion of the amino acid sequence of the antigen, i.e., CLDN6. In other various embodiments, the epitope is a conformational or structural epitope. By “conformational epitope” or “structural epitope” is meant an epitope which is composed of amino acids which are located in close proximity to one another only when the CLDN6 is in its properly folded state. Unlike linear epitopes, the amino acids of a conformational or structural epitope are not adjacent to each other in the primary structure (i.e., amino acid sequence) of the CLDN6. A conformational or structural epitope is not made of contiguous amino acids of the amino acid sequence of the antigen (CLDN6).

In various aspects, the epitope is located within the extracellular domain (ECD) of CLDN6, e.g., human CLDN6. In various aspects, the antigen binding protein binds to Extracellular Loop 2 (EL2) of the ECD of CLDN6 having the amino acid sequence of WTAHAIIRDFYNPLVAEAQKREL (SEQ ID NO: 2). In various aspects, the epitope to which the antigen-binding protein binds is within SEQ ID NO: 2. In various aspects, the antigen-binding protein of the present disclosure binds to an N-terminal portion of SEQ ID NO: 2, e.g., TAHAIIRDFYNPL (SEQ ID NO: 3). In various aspects, the antigen-binding protein of the present disclosure binds to a C-terminal portion of SEQ ID NO: 2, e.g., LVAEAQKREL (SEQ ID NO: 4). In various instances, the antigen-binding protein of the present disclosure binds to EL2, but not to Extracellular Loop 1 (EL1) of CLDN6. In various aspects, the epitope(s) to which the antigen binding proteins of the present disclosure bind to is different from the epitope bound by an anti-CLDN6 antibody comprising a light chain variable region comprising the sequence of SEQ ID NO: 185 and a heavy chain variable region comprising the sequence of SEQ ID NO: 186. In various aspects, the epitope(s) to which the antigen binding proteins of the present disclosure bind to is different from the epitope bound by an anti-CLDN6 antibody comprising a light chain variable region comprising the sequence of SEQ ID NO: 181 and a heavy chain variable region comprising the sequence of SEQ ID NO: 182.

In various aspects, the antigen-binding proteins bind to human CLDN6 and a non-human CLDN6. In various instances, the non-human CLDN6 is a CLDN6 of chimpanzee, Rhesus monkey, dog, cow, mouse, rat, zebrafish, or frog. In various instances, the antigen-binding proteins bind to human CLDN6 and mouse CLDN6.

### Affinity and Avidity

The antigen-binding proteins provided herein bind to CLDN6 in a non-covalent and reversible manner. In various embodiments, the binding strength of the antigen-binding protein to CLDN6 may be described in terms of its affinity, a measure of the strength of interaction between the binding site of the antigen-binding protein and the epitope. In various aspects, the antigen-binding proteins provided herein have high-affinity for CLDN6 and thus will bind a greater amount of CLDN6 in a shorter period of time than low-affinity antigen-binding proteins. In various aspects, the antigen-binding protein has an equilibrium association constant, K_{A}, which is at least 10⁵ M⁻¹, at least 10⁶ M⁻¹, at least 10⁷ M⁻¹, at least 10⁸ M⁻¹, at least 10⁹ M⁻¹, or at least 10¹⁰ M⁻¹. As understood by the artisan of ordinary skill, K_{A} can be influenced by factors including pH, temperature and buffer composition.

In various embodiments, the binding strength of the antigen-binding protein to CLDN6 may be described in terms of its sensitivity. K_{D} is the equilibrium dissociation constant, a ratio of k_{off}/kₒₙ, between the antigen-binding protein and CLDN6. K_{D} and K_{A} are inversely related. The K_{D} value relates to the concentration of the antigen-binding protein (the amount of antigen-binding protein needed for a particular experiment) and so the lower the K_{D} value (lower concentration) the higher the affinity of the antigen-binding protein. In various aspects, the binding strength of the antigen-binding protein to CLDN6 may be described in terms of K_{D}. In various aspects, the K_{D} of the antigen-binding proteins provided herein is about 10⁻¹ M, about 10⁻² M, about 10⁻³ M, about 10⁻⁴ M, about 10⁻⁵ M, about 10⁻⁶ M, or less. In various aspects, the K_{D} of the antigen-binding proteins provided herein is micromolar, nanomolar, picomolar or femtomolar. In various aspects, the K_{D} of the antigen-binding proteins provided herein is within a range of about 10⁻⁴ M to 10⁻⁶ M, or 10⁻⁷ M to 10⁻⁹ M, or 10⁻¹⁰ M to 10⁻¹² M, or 10⁻¹³ M to 10⁻¹⁵ M. In various aspects, the K_{D} of the antigen-binding proteins provided herein is within a range of about 1.0×10⁻¹² M to about 1.0×10⁻⁸ M. In various aspects, the K_{D} of the antigen-binding proteins is within a range of about 1.0×10⁻¹¹ M to about 1.0×10⁻⁹ M.

In various aspects, the affinity of the antigen-binding proteins are measured or ranked using a flow cytometry- or Fluorescence-Activated Cell Sorting (FACS)-based assay. Flow cytometry-based binding assays are known in the art. See, e.g., Cedeno-Arias et al., Sci Pharm 79(3): 569-581 (2011); Rathanaswami et al., Analytical Biochem 373: 52-60 (2008); and Geuijen et al., J Immunol Methods 302(1-2): 68-77 (2005). In various aspects, the affinity of the antigen-binding proteins are measured or ranked using a competition assay as described in Trikha et al., Int J Cancer 110: 326-335 (2004) and Tam et al., Circulation 98(11): 1085-1091 (1998), as well as below. See section titled “Competition Assays” below. In Trikh et al., cells that express the antigen were used in a radioassay. The binding of ¹²⁵I-labeled antigen-binding protein (e.g., antibody) to the cell surface antigen is measured with the cells in suspension. In various aspects, the relative affinity of a CLDN6 antibody is determined via a FACS-based assay in which different concentrations of a CLDN6 antibody conjugated to a fluorophore are incubated with cells expressing CLDN6 and the fluorescence emitted (which is a direct measure of antibody-antigen binding) is determined. A curve plotting the fluorescence for each dose or concentration is made. The max value is the lowest concentration at which the fluorescence plateaus or reaches a maximum, which is when binding saturation occurs. Half of the max value is considered an EC50 or an IC50 and the antibody with the lowest EC50/IC50 is considered as having the highest affinity relative to other antibodies tested in the same manner.

In various aspects, the IC₅₀ value, as determined in a competitive binding inhibition assay, approximates the K_{D} of the antigen-binding protein. In various instances, as discussed below, the competition assay is a FACS-based assay carried out with a reference antibody, fluorophore-conjugated secondary antibody, and cells which express CLDN6. In various aspects, the cells are genetically-engineered to overexpress CLDN6. In some aspects, the cells are HEK293T cells transduced with a viral vector to express CLDN6. In alternative aspects, the cells endogenously express CLDN6. Before the FACS-based assay is carried out, in some aspects, the cells which endogenously express CLDN6 are pre-determined as low CLDN6-expressing cells or high CLDN6-expressing cells. In some aspects, the cells are cancer or tumor cells. In various aspects, the cells are cells from a cell line, e.g., an ovarian cell line, endometrial cell line, bladder cell line, lung cell line, gastrointestinal (GI) cell line, liver cell line, lung cell line, and the like. In various aspects, the cells which endogenously express CLDN6 as selected from the group consisting of OVCA429 ovarian cells, ARK2 endometrial cells, OAW28 ovarian cells, UMUC-4 bladder cells, PEO14 ovarian cells, OV177 ovarian cells, H1693 lung cells, MKN7 upper GI cells, OV-90 ovarian cells, HUH-7 liver cells, JHOS-4 ovarian cells, H1435 lung cells, and NUGC3 upper GI cells. In various aspects, the antigen-binding protein inhibits the binding interaction between human CLDN6 expressed by the cells and the reference antibody, which reference antibody is known to bind to CLDN6 but is not an antigen-binding protein of the present disclosure. In various instances, the antigen-binding proteins of the present disclosure compete with the reference antibody for binding to human CLDN6 and thereby reduce the amount of human CLDN6 bound to the reference antibody as determined by an in vitro competitive binding assay. In various aspects, the antigen-binding proteins of the present disclosure inhibit the binding interaction between human CLDN6 and the reference antibody and the inhibition is characterized by an IC₅₀. In various aspects, the antigen-binding proteins exhibit an IC₅₀ of less than about 2500 nM for inhibiting the binding interaction between human CLDN6 and the reference antibody. In various aspects, the antigen-binding proteins exhibit an IC₅₀ of less than about 2000 nM, less than about 1500 nM, less than about 1000 nM, less than about 900 nM, less than about 800 nM, less than about 700 nM, less than about 600 nM, less than about 500 nM, less than about 400 nM, less than about 300 nM, less than about 200 nM, or less than about 100 nM. In various aspects, the antigen-binding proteins exhibit an IC₅₀ of less than about 90 nM, less than about 80 nM, less than about 70 nM, less than about 60 nM, less than about 50 nM, less than about 40 nM, less than about 30 nM, less than about 20 nM, or less than about 10 nM. In various instances, the antigen binding proteins of the present disclosure compete against a reference antibody known to bind to CLDN6 (which reference antibody is different from any of the antigen-binding proteins of the present disclosure) for binding to CLDN6. See further description under Competition assays.

Avidity gives a measure of the overall strength of an antibody-antigen complex. It is dependent on three major parameters: affinity of the antigen-binding protein for the epitope, valency of both the antigen-binding protein and CLDN6, and structural arrangement of the parts that interact. The greater an antigen-binding protein's valency (number of antigen binding sites), the greater the amount of antigen (CLDN6) it can bind. In various aspects, the antigen-binding proteins have a strong avidity for CLDN6. In various aspects, the antigen-binding proteins are multivalent. In various aspects, the antigen-binding proteins are bivalent. In various instances, the antigen antigen-binding proteins are monovalent.

### Cross-Reactivity

In various embodiments, the antibodies of the present disclosure bind to CLDN6 and do not bind to any other member of the CLDN family, e.g., do not cross-react with any other member of the CLDN family. In various instances, the antibodies of the present disclosure are CLDN-6 specific. In various embodiments, the antibodies of the present disclosure have a selectivity for CLDN6 which is at least 10-fold, 5-fold, 4-fold, 3-fold, 2-fold greater than the selectivity of the antibodies for CLDN3, CLDN4, CLDN9, or a combination thereof. In various embodiments, the antibodies of the present disclosure have a selectivity for CLDN6 which is at least 10-fold, 5-fold, 4-fold, 3-fold, 2-fold greater than the selectivity of the antibodies for each of CLDN3, CLDN4, and CLDN9. Selectivity may be based on the K_{D} exhibited by the antibodies for CLDN6, or a CLDN family member, wherein the K_{D} may be determined by techniques known in the art, e.g., surface plasmon resonance, FACS-based affinity assays.

In various aspects, the antibodies of the present disclosure bind to CLDN6 and do not bind to any of Claudin3 (CLDN3), Claudin4 (CLDN4), and Claudin9 (CLDN9). In various aspects, the antibodies do not bind to any of CLDN3, CLDN4, and CLDN9 and exhibit an IC₅₀ of less than about 1200 nM (e.g., less than about 1000 nM, less than about 750 nM, less than about 500 nM, less than about 250 nM) in a FACS-based assay with OVCA429 cells endogenously expressing CLDN6. In various aspects, the antibodies do not bind to any of CLDN3, CLDN4, and CLDN9 and the concentration at which 50% of binding saturation is achieved with OVCA429 cells endogenously expressing CLDN6 is less than about 1200 nM (e.g., less than about 1000 nM, less than about 750 nM, less than about 500 nM, less than about 250 nM). In various aspects, the antibodies exhibit at least a 5-fold selectivity for CLDN 6 greater than that for CLDN3, CLDN4, and CLDN9 and the concentration at which 50% of binding saturation is achieved with OVCA429 cells endogenously expressing CLDN6 is less than about 1200 nM (e.g., less than about 1000 nM, less than about 750 nM, less than about 500 nM, less than about 250 nM). In various aspects, the antibodies exhibit an IC50 of less than about 1200 nM (e.g., less than about 1000 nM, less than about 750 nM, less than about 500 nM, less than about 250 nM) for CLDN6 artificial and endogenous models and exhibit a greater than about 5-fold ratio separating CLDN6 IC50s from CLDN3, CLDN4 and/or CLDN9. In various instances, the antibodies exhibit an IC50 of less than about 1200 nM (e.g., less than about 1000 nM, less than about 750 nM, less than about 500 nM, less than about 250 nM) for CLDN6 and exhibit an IC50 for any one of CLDN3, CLDN4, and CLDN9 at least 5-fold greater than the IC50.

### Competition Assays

In various embodiments, the antigen-binding protein inhibits a binding interaction between human CLDN6 and a reference antibody, which reference antibody is known to bind to CLDN6 but is not an antigen-binding protein of the present disclosure. In various instances, the antigen-binding proteins of the present disclosure compete with the reference antibody for binding to human CLDN6 and thereby reduce the amount of human CLDN6 bound to the reference antibody as determined by an in vitro competitive binding assay. In various embodiments, the reference antibody binds to an epitope within the amino acid sequence of the extracellular domain of human CLDN6, optionally, within EL2 or EL1. In various aspects, the reference antibody comprises a light chain variable sequence encoded by SEQ ID NO: 179, and a heavy chain variable sequence encoded by SEQ ID NO: 180. In various aspects, the reference antibody comprises a light chain variable sequence of SEQ ID NO: 181, and a heavy chain variable sequence of SEQ ID NO: 182. In various aspects, the antigen-binding proteins of the present disclosure inhibit the binding interaction between human CLDN6 and the reference antibody and the inhibition is characterized by an IC₅₀. In various aspects, the antigen-binding proteins exhibit an IC₅₀ of less than about 2500 nM for inhibiting the binding interaction between human CLDN6 and the reference antibody. In various aspects, the antigen-binding proteins exhibit an IC₅₀ of less than about 2000 nM, less than about 1500 nM, less than about 1000 nM, less than about 900 nM, less than about 800 nM, less than about 700 nM, less than about 600 nM, less than about 500 nM, less than about 400 nM, less than about 300 nM, less than about 200 nM, or less than about 100 nM. In various aspects, the antigen-binding proteins exhibit an IC₅₀ of less than about 90 nM, less than about 80 nM, less than about 70 nM, less than about 60 nM, less than about 50 nM, less than about 40 nM, less than about 30 nM, less than about 20 nM, or less than about 10 nM.

In various instances, the antigen-binding proteins of the present disclosure compete with the reference antibody for binding to human CLDN6 and thereby reduce the amount of human CLDN6 bound to the reference antibody as determined by an in vitro competitive binding assay. In various aspects, the in vitro competitive binding assay is a FACS-based assay in which the fluorescence of a fluorophore-conjugated secondary antibody which binds to the Fc of the reference antibody is measured in the absence or presence of a particular amount of the antigen-binding protein of the present disclosure. In various aspects, the FACS-based assay is carried out with the reference antibody, fluorophore-conjugated secondary antibody and cells which express CLDN6. In various aspects, the cells are genetically-engineered to overexpress CLDN6. In some aspects, the cells are HEK293T cells transduced with a viral vector to express CLDN6. In alternative aspects, the cells endogenously express CLDN6. Before the FACS-based assay is carried out, in some aspects, the cells which endogenously express CLDN6 are pre-determined as low CLDN6-expressing cells or high CLDN6-expressing cells. In some aspects, the cells are cancer or tumor cells. In various aspects, the cells are cells from a cell line, e.g., an ovarian cell line, endometrial cell line, bladder cell line, lung cell line, gastrointestinal (GI) cell line, liver cell line, lung cell line, and the like. In various aspects, the cells which endogenously express CLDN6 as selected from the group consisting of OVCA429 ovarian cells, ARK2 endometrial cells, OAW28 ovarian cells, UMUC-4 bladder cells, PEO14 ovarian cells, OV177 ovarian cells, H1693 lung cells, MKN7 upper GI cells, OV-90 ovarian cells, HUH-7 liver cells, JHOS-4 ovarian cells, H1435 lung cells, and NUGC3 upper GI cells. In various instances, the antigen binding proteins of the present disclosure bind to CLDN6 endogenously expressed by one or more of ARK2 cells, OVCA429 cells, LS513 cells, or MCF7 cells with high affinity. In various aspects, the antigen binding proteins exhibit an IC₅₀ of less than about 3000 nM as determined in a FACS-based competitive binding inhibition assay using one or more of ARK2 cells, OVCA429 cells, LS513 cells, or MCF7 cells. In various aspects, the antigen binding proteins exhibit an IC₅₀ of less than about 2500 nM, less than about 2000 nM, less than about 1750 nM, less than about 1500 nM, less than about 1250 nM, less than about 1000 nM, less than about 750 nM, or less than about 500 nM, as determined in a FACS-based competitive binding inhibition assay using one or more of ARK2 cells, OVCA429 cells, LS513 cells, or MCF7 cells. In various aspects, the antigen binding proteins exhibit an IC₅₀ of less than about 400 nM, less than about 300 nM, less than about 200 nM, less than about 100 nM, less than about 75 nM, less than about 50 nM, less than about 25 nM, or less than about 10 nM, as determined in a FACS-based competitive binding inhibition assay using one or more of ARK2 cells, OVCA429 cells, LS513 cells, or MCF7 cells.

Other binding assays, e.g., competitive binding assays or competition assays, which test the ability of an antibody to compete with a second antibody for binding to an antigen, or to an epitope thereof, are known in the art. See, e.g., Trikha et al., Int J Cancer 110: 326-335 (2004); Tam et al., Circulation 98(11): 1085-1091 (1998). U.S. Patent Application Publication No. US20140178905, Chand et al., Biologicals 46: 168-171 (2017); Liu et al., Anal Biochem 525: 89-91 (2017); and Goolia et al., J Vet Diagn Invest 29(2): 250-253 (2017). Also, other methods of comparing two antibodies are known in the art, and include, for example, surface plasmon resonance (SPR). SPR can be used to determine the binding constants of the antibody and second antibody and the two binding constants can be compared.

### Methods of Antibody Production and Related Methods

Suitable methods of making antibodies are known in the art. For instance, standard hybridoma methods for producing antibodies are described in, e.g., Harlow and Lane (eds.), Antibodies: A Laboratory Manual, CSH Press (1988), and CA. Janeway et al. (eds.), Immunobiology, 5^{th} Ed., Garland Publishing, New York, NY (2001)). In certain embodiments, methods of producing antibodies and conjugates for use in the methods disclosed herein are described in US2022372134, US2022040321, and US2023049752, each of which is incorporated herein by reference in its entirety.

Depending on the host species, various adjuvants can be used to increase the immunological response leading to greater antibody production by the host. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Other methods of antibody production are summarized in Table 1.

TABLE 1

| Technique | Various references |
|---|---|
| EBV-hybridoma methods and | Haskard and Archer, J. |
| Bacteriophage vector expression | Immunol. Methods, 74(2), |
| systems | 361-67 (1984), Roder et al., |
| | Methods Enzymol., 121, |
| | 140-67 (1986), and Huse |
| | et al., Science, 246, |
| | 1275-81 (1989)). |
| methods of producing antibodies | U.S. Pat. Nos. 5,545,806, and |
| in non-human animals | 5,714,352, and U.S. patent |
| | application publication No. |
| | 2002/0197266 |
| inducing in vivo production in | Orlandi et al (Proc Natl Acad |
| the lymphocyte population or | Sci 86: 3833-3837; 1989), |
| by screening recombinant | and Winter G and Milstein |
| immunoglobulin libraries or | C (Nature 349: 293-299, |
| panels of highly specific | 1991). |
| binding reagents | |
| methods of producing | “Protein production and |
| recombinant proteins | purification” Nat Methods |
| | 5(2): 135-146 (2008). |
| Phage display | Janeway et al., supra, Huse |
| | et al., supra, and U.S. Pat. |
| | No. 6,265,150). Related |
| | methods also are described |
| | in U.S. Pat. No. 5,403,484; |
| | U.S. Pat. No. 5,571,698; |
| | U.S. Pat. No. 5,837,500 |
| | U.S. Pat. No. 5,702,892. |
| | The techiniques described |
| | in U.S. Pat. No. 5,780,279; |
| | U.S. Pat. No. 5,821,047; |
| | U.S. Pat. No. 5,824,520; |
| | U.S. Pat. No. 5,855,885; |
| | U.S. Pat. No. 5,858,657; |
| | U.S. Pat. No. 5,871,907; |
| | U.S. Pat. No. 5,969,108; |
| | U.S. Pat. No. 6,057,098; |
| | and U.S. Pat. No. 6,225,447 |
| Antibodies can be produced | U.S. Pat. Nos. 5,545,806 and |
| by transgenic mice | 5,569,825, and Janeway et |
| | al., supra. |

Methods of testing antibodies for the ability to bind to the epitope of CLDN6 regardless of how the antibodies are produced are known in the art and include any antibody-antigen binding assay, such as, for example, radioimmunoassay (RIA), ELISA, Western blot, immunoprecipitation, SPR, and competitive inhibition assays (see, e.g., Janeway et al., infra, and U.S. Patent Application Publication No. 2002/0197266, and the above section relating to competition assays).

### Sequences/Structure

CLND6 binding proteins are known in the art. For example, US2022372134 and US2023049752, each of which is hereby incorporated by reference in its entirety, describe CLDN6 binding proteins. CLDN6 antigen binding proteins are also described in US20220168438, US20200261594, US20220168440, US20220125943 (for example, SEQ ID NOS: 19, 21, 23, and 25 from this publication), US2020339677 (for example, SEQ ID NOS: 34-41 and FIGS. 25-26 from this publication), US2021179730 (for example, SEQ ID NOS: 34-41, FIGS. 25-26 from this publication), US2021079113, US2020385460 (for example, SEQ ID NOS: 3-12 from this publication), US2020399370, US2022306711, US2022162302 (for example, SEQ ID NOS: 40-45 from this publication), US2019309067, and WO2022187275 (for example, SEQ ID NOS: 1-4 and 23-31 from this publication), each of which is hereby incorporated by reference in its entirety. Sequences are listed with SEQ ID NOS in the Sequence Listing. Provided herein are antigen-binding proteins comprising (a) a heavy chain (HC) complementarity-determining region (CDR) 1 amino acid sequence set forth in Table A or a sequence selected from the group consisting of: SEQ ID NOs: 11, 17, 23, 29, 35, 41, 47, 53, 59, 65, 71, 107, 125, and 131, or a variant sequence thereof which differs by only one or two amino acids or which has at least or about 70% (e.g., at least about 80%, at least about 85%, at least about 90%, at least about 95%) sequence identity; (b) an HC CDR2 amino acid sequence set forth in Table A or a sequence selected from the group consisting of: SEQ ID NOs: 12, 18, 24, 30, 36, 42, 48, 54, 60, 66, 72, 108, 126, and 132, or a variant sequence thereof which differs by only one or two amino acids or which has at least or about 70% (e.g., at least about 80%, at least about 85%, at least about 90%, at least about 95%) sequence identity; (c) an HC CDR3 amino acid sequence set forth in Table A or a sequence selected from the group consisting of: SEQ ID NOs: 13, 19, 25, 31, 37, 43, 49, 55, 61, 67, 73, 109, 127, and 133, or a variant sequence thereof which differs by only one or two amino acids or which has at least or about 70% (e.g., at least about 80%, at least about 85%, at least about 90%, at least about 95%) sequence identity; (d) a light chain (LC) CDR1 amino acid sequence set forth in Table A or a sequence selected from the group consisting of: SEQ ID NOs: 8, 14, 20, 32, 38, 44, 50, 56, 62, 68, 104, 122, and 128, or a variant sequence thereof which differs by only one or two amino acids or which has at least or about 70% (e.g., at least about 80%, at least about 85%, at least about 90%, at least about 95%) sequence identity; (e) an LC CDR2 amino acid sequence set forth in Table A or a sequence selected from the group consisting of: SEQ ID NOs: 9, 15, 21, 27, 33, 39, 45, 51, 57, 63, 69, 105, 123, and 129, or a variant sequence thereof which differs by only one or two amino acids or which has at least or about 70% (e.g., at least about 80%, at least about 85%, at least about 90%, at least about 95%) sequence identity; (f) an LC CDR3 amino acid sequence set forth in Table A or a sequence selected from the group consisting of: SEQ ID NOs: 10, 16, 22, 28, 34, 40, 46, 52, 58, 64, 70, 106, 124, and 130, or a variant sequence thereof which differs by only one or two amino acids or which has at least or about 70% (e.g., at least about 80%, at least about 85%, at least about 90%, at least about 95%) sequence identity; or (g) a combination of any two or more of (a)-(f).

TABLE A

| | LC CDR1 | LC CDR2 | LC CDR3 | HC CDR1 | HC CDR2 | HC CDR3 |
|---|---|---|---|---|---|---|
| AB1 | 8 | 9 | 10 | 11 | 12 | 13 |
| AB2 | 14 | 15 | 16 | 17 | 18 | 19 |
| AB3 | 20 | 21 | 22 | 23 | 24 | 25 |
| AB4 | 26 | 27 | 28 | 29 | 30 | 31 |
| AB5 | 32 | 33 | 34 | 35 | 36 | 37 |
| AB6 | 38 | 39 | 40 | 41 | 42 | 43 |
| AB7 | 44 | 45 | 46 | 47 | 48 | 49 |
| AB8 | 50 | 51 | 52 | 53 | 54 | 55 |
| AB9 | 56 | 57 | 58 | 59 | 60 | 61 |
| AB10 | 62 | 63 | 64 | 65 | 66 | 67 |
| AB11 | 68 | 69 | 70 | 71 | 72 | 73 |
| AB17 | 104 | 105 | 106 | 107 | 108 | 109 |
| AB20 | 122 | 123 | 124 | 125 | 126 | 127 |
| AB21 | 128 | 129 | 130 | 131 | 132 | 133 |
| AB-D1 | Dx4 | Dx5 | Dx6 | Dx1 | Dx2 | Dx3 |
| AB-D2 | Dx10 | Dx11 | Dx12 | Dx7 | Dx8 | Dx9 |
| AB-D3 | Dx16 | Dx17 | Dx18 | Dx13 | Dx14 | Dx15 |
| AB-D4 | Dx22 | Dx23 | Dx24 | Dx19 | Dx20 | Dx21 |

SEQ ID NOs:

Dx1 (SEQ ID NO: 514):
Gly Tyr Thr Phe Thr Glu Tyr Thr Met His
Dx2 (SEQ ID NO: 515):
Gly Val Asn Pro Asn Ser Gly Asp Thr Ser
Dx3 (SEQ ID NO: 516):
Pro Gly Gly Tyr Asp Val Gly Tyr Tyr Ala Met Asp
Tyr
Dx4 (SEQ ID NO: 517):
Arg Ala Ser Gln Asp Ile Asn Asn Tyr Leu Asn
Dx5 (SEQ ID NO: 518):
Phe Thr Ser Arg Leu His Ser
Dx6 (SEQ ID NO: 519):
Gln Gln Gly Tyr Pro Leu Pro Trp Thr
Dx7 (SEQ ID NO: 520):
Gly Tyr Thr Phe Thr Glu Tyr Thr Met His
Dx8 (SEQ ID NO: 521):
Gly Val Asn Pro Asn Ser Gly Asp Thr Ser
Dx9 (SEQ ID NO: 522):
Pro Gly Gly Tyr Asp Val Gly Tyr Tyr Ala Met Asp
Tyr
Dx10 (SEQ ID NO: 523):
Arg Ala Ser Gln Asp Ile Asn Asn Tyr Leu Asn
Dx11 (SEQ ID NO: 524):
Ser Thr Ser Arg Leu His Ser
Dx12 (SEQ ID NO: 525):
Gln Gln Gly Tyr Pro Leu Pro Trp Thr
Dx13 (SEQ ID NO: 526):
Gly Tyr Thr Phe Thr Glu Tyr Thr Met His
Dx14 (SEQ ID NO: 527):
Gly Val Asn Pro Asn Ser Gly Asp Thr Ser
Dx15 (SEQ ID NO: 528):
Pro Gly Gly Tyr Asp Val Gly Tyr Tyr Ala Met Asp
Tyr
Dx16 (SEQ ID NO: 529):
Arg Ala Ser Gln Asp Ile Asn Asn Tyr Leu Asn
Dx17 (SEQ ID NO: 530):
Phe Thr Ser Arg Leu His Ser
Dx18 (SEQ ID NO: 531):
Gln Gln Gly Tyr Pro Leu Pro Trp Thr
Dx19 (SEQ ID NO: 532):
Gly Tyr Thr Phe Thr Glu Tyr Thr Met His
Dx20 (SEQ ID NO: 533):
Gly Val Asn Pro Asn Ser Gly Asp Thr Ser
Dx21 (SEQ ID NO: 534):
Pro Gly Gly Tyr Asp Val Gly Tyr Tyr Ala Met Asp
Tyr
Dx22 (SEQ ID NO: 535):
Arg Ala Ser Gln Asp Ile Asn Asn Tyr Leu Asn
Dx23 (SEQ ID NO: 536):
Ser Thr Ser Arg Leu His Ser
Dx24 (SEQ ID NO: 537):
Gln Gln Gly Tyr Pro Leu Pro Trp Thr

In various aspects, the antigen-binding protein comprises a LC CDR1 amino acid sequence, a LC CDR2 amino acid sequence, and a LC CDR3 amino acid sequence set forth in Table A and at least 1 or 2 of the HC CDR amino acid sequences set forth in Table A. In various aspects, the antigen-binding protein comprises a HC CDR1 amino acid sequence, a HC CDR2 amino acid sequence, and a HC CDR3 amino acid sequence set forth in Table A and at least 1 or 2 of the LC CDR amino acid sequences set forth in Table A.

In various embodiments, the antigen-binding protein comprises at least 3, 4, or 5 of the amino acid sequences designated by the SEQ ID NOs: in a single row of Table A. In various embodiments, the antigen-binding protein comprises each of the LC CDR amino acid sequences designated by the SEQ ID NOs: of a single row of Table A and at least 1 or 2 of the HC CDR amino acid sequences designated by the SEQ ID NOs: in of a single row of Table A. In various embodiments, the antigen-binding protein comprises each of the HC CDR amino acid sequences designated by the SEQ ID NOs: of a single row of Table A and at least 1 or 2 of the LC CDR amino acid sequences designated by the SEQ ID NOs: of a single row of Table A. In various embodiments, the antigen-binding protein comprises all 6 of the CDR amino acid sequences designated by the SEQ ID NOs: of a single row of Table A. In various embodiments, the antigen-binding protein comprises six CDR amino acid sequences selected from the group consisting of: SEQ ID NOs: 50-55; SEQ ID NOs: 122-127; SEQ ID NOs: 26-31; SEQ ID NOs: 128-133; SEQ ID NOs: 38-43; SEQ ID NOs: 62-67; SEQ ID NOs: 44-49; SEQ ID NOs: 104-109; SEQ ID NOs: 56-61; SEQ ID NOs: 32-37; SEQ ID NOs: 8-13; SEQ ID NOs: 68-73; SEQ ID NOs: 14-19; and SEQ ID NOs: 20-25.

In various instances, the amino acid sequences of Table A are separated by at least one or more (e.g., at least 2, 3, 4, 5, 6, 7, 8, 9, 10, or more) intervening amino acid(s). In various instances, there are about 10 to about 20 amino acids between the sequences of the LC CDR1 and the LC CDR2 and about 25 to about 40 amino acids between the sequences of the LC CDR2 and the LC CDR3. In various instances, there are about 14 to about 16 amino acids between the sequences of the LC CDR1 and the LC CDR2 and about 30 to about 35 amino acids between the sequences of LC CDR2 and the LC CDR3. In various instances, there are about 10 to about 20 amino acids between the sequences of the HC CDR1 and HC CDR2 and about 25 to about 40 amino acids between the sequences of the HC CDR2 and the HC CDR3. In various instances, there are about 14 to about 16 amino acids between the sequences of the HC CDR1 and HC CDR2 and about 30 to about 35 amino acids between the sequences of the HC CDR2 and HC CDR3.

In various embodiments, the antigen-binding protein comprises (a) a heavy chain variable region amino acid sequence set forth in in Table B or a sequence selected from the group consisting of: SEQ ID NOs: 135, 137, 139, 141, 143, 145, 147, 149, 151, 153, 155, 167, 173, and 175, or a variant sequence thereof which differs by only one or two amino acids or which has at least or about 70% (e.g., at least about 80%, at least about 85%, at least about 90%, at least about 95%) sequence identity; or (b) a light chain variable region amino acid sequence set forth in Table B or a sequence selected from the group consisting of: SEQ ID NOs: 134, 136, 138, 140, 142, 144, 146, 148, 150, 152, 154, 166, 172, 174, and 176, or a variant sequence thereof which differs by only one or two amino acids or which has at least or about 70% (e.g., at least about 80%, at least about 85%, at least about 90%, at least about 95%) sequence identity; or (c) both (a) and (b).

TABLE B

| | Light Chain | Heavy Chain |
|---|---|---|
| | Variable | Variable |
| | Region | Region |
| AB1 | 134 | 135 |
| AB2 | 136 | 137 |
| AB3 | 138 | 139 |
| AB4 | 140 | 141 |
| AB5 | 142 | 143 |
| AB6 | 144 | 145 |
| AB7 | 146 | 147 |
| AB8 | 148 | 149 |
| AB9 | 150 | 151 |
| AB10 | 152 | 153 |
| AB11 | 154 | 155 |
| AB17 | 166 | 167 |
| AB20 | 172 | 173 |
| AB21 | 174 | 175 |

In various embodiments, the antigen-binding protein comprises a pair of amino acid sequences selected from the group consisting of: SEQ ID NOs: 148 and 149; SEQ ID NOs: 172 and 173; SEQ ID NOs: 140 and 141; SEQ ID NOs: 174 and 175; SEQ ID NOs: 144 and 145; SEQ ID NOs: 152 and 153; SEQ ID NOs: 146 and 147; SEQ ID NOs: 166 and 167; SEQ ID NOs: 150 and 151; SEQ ID NOs: 142 and 143; SEQ ID NOs: 164 and 165; SEQ ID NOs: 162 and 163; SEQ ID NOs: 134 and 135; SEQ ID NOs: 154 and 155; SEQ ID NOs: 136 and 137; and SEQ ID NOs: 138 and 139.

In various aspects, the antigen-binding protein comprises an amino acid sequence which is similar to an above-referenced amino acid sequence, yet the antigen-binding protein substantially retains its biological function, e.g., its ability to bind to human CLDN6, reduce tumor growth, inhibit tumor growth, and/or treat cancer.

In various aspects, the antigen-binding protein comprises an amino acid sequence which differs by only 1, 2, 3, 4, 5, 6, or more amino acids, relative to the above-referenced amino acid sequence(s). In various aspects, the antigen-binding protein comprises a variant sequence of the referenced sequence, which variant sequence differs by only one or two amino acids, relative to the referenced sequence. In various aspects, the antigen-binding protein comprising one or more amino acid substitutions that occur outside of the CDRs, e.g., the one or more amino acid substitutions occur within the framework region(s) of the heavy or light chain. In various aspects, the antigen-binding protein comprising one or more amino acid substitutions yet the antigen-binding protein retains the amino acid sequences of the six CDRs. In various aspects, the antigen-binding protein comprises an amino acid sequence having only 1, 2, 3, 4, 5, 6, or more conservative amino acid substitutions, relative to the above-referenced amino acid sequence(s). As used herein, the term “conservative amino acid substitution” refers to the substitution of one amino acid with another amino acid having similar properties, e.g., size, charge, hydrophobicity, hydrophilicity, and/or aromaticity, and includes exchanges within one of the following five groups:
I. Small aliphatic, nonpolar or slightly polar residues:
   Ala, Ser, Thr, Pro, Gly;
II. Polar, negatively charged residues and their amides and esters:
   Asp, Asn, Glu, Gln, cysteic acid and homocysteic acid;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine

In various aspects, the conservative amino acid substitution is an exchange within one of the following groups of amino acids:
I. aliphatic amino acids: Gly, Ala, Val, Leu, Ile
II. non-aromatic amino acids comprising a side chain hydroxyl: Ser, Thr
III. amino acids comprising a sulfur side chain: Cys, Met
IV: amino acids comprising a side chain aromatic ring: Phe, Tyr, Trp
V: acidic amino acid: Glu; Asp
VI: basic amino acid: Arg; Lys
VII: amino acid comprising a side chain amide: Gln, Asn
VIII: amino acid comprising a side chain imidazole: His, alpha-dimethyl imidiazole acetic acid (DMIA)
IX: imino acid: Pro, 4-hydroxy-Pro, 4-amino-Pro

In various aspects, the antigen-binding protein comprises an amino acid sequence which has greater than or about 30%, greater than or about 50%, or greater than or about 70% sequence identity to the above-referenced amino acid sequence. In various aspects, the antigen-binding protein comprises an amino acid sequence which has at least 30%, at least 40%, at least 50%, at least 60%, at least 70%, at least 80%, at least 85%, at least 90% or has greater than 90% sequence identity to the above-referenced amino acid sequence. In various aspects, the antigen-binding protein comprises an amino acid sequence that has at least 70%, at least 80%, at least 85%, at least 90% or has greater than 90% sequence identity along the full-length of the above-referenced amino acid sequence. In various aspects, the antigen-binding protein comprises an amino acid sequence having at least 95%, 96%, 97%, 98% or 99% sequence identity along the full-length of the above-referenced amino acid sequence.

In various aspects, the antigen-binding protein comprises a variant sequence of the referenced sequence, which variant sequence has at least or about 70% sequence identity, relative to the above-referenced sequence. In various aspects, the antigen-binding protein comprises a variant sequence of the referenced sequence, which variant sequence has at least or about 80% sequence identity, relative to the above-referenced sequence. In various aspects, the antigen-binding protein comprises a variant sequence of the referenced sequence, which variant sequence has at least or about 90% sequence identity, relative to the above-referenced sequence. In various aspects, the antigen-binding protein comprises a variant sequence of the referenced sequence, which variant sequence has at least or about 95% sequence identity, relative to the above-referenced sequence.

In various embodiments, the antigen-binding protein comprises one, two, three, four, or five sequences of the SEQ ID NOs. in a single row of Table A and at least one variant sequence having at least or about 70% (e.g., at least about 80%, at least about 85%, at least about 90%, at least about 95%) sequence identity to any of SEQ ID NOs: 8-133. In various embodiments, the antigen-binding protein comprises one, two, three, four, or five sequences of a set of sequences selected from: SEQ ID NOs: 50-55; SEQ ID NOs: 122-127; SEQ ID NOs: 26-31; SEQ ID NOs: 128-133; SEQ ID NOs: 38-43; SEQ ID NOs: 62-67; SEQ ID NOs: 44-49; SEQ ID NOs: 104-109; SEQ ID NOs: 56-61; SEQ ID NOs: 32-37; SEQ ID NOs: 8-13; SEQ ID NOs: 68-73; SEQ ID NOs: 14-19; and SEQ ID NOs: 20-25, wherein the antigen-binding protein further comprises at least one variant sequence having at least or about 70% (e.g., at least about 80%, at least about 85%, at least about 90%, at least about 95%) sequence identity to at least one of the sequences of the set.

In various embodiments, the antigen-binding protein comprises a pair of variant sequences having at least or about 70% (e.g., at least about 80%, at least about 85%, at least about 90%, at least about 95%) sequence identity to any of SEQ ID NOs: 134-155 and 166-167 and 172-175. In various instances, the antigen binding protein comprises a pair of variant sequences which have at least or about 70% (e.g., at least about 80%, at least about 85%, at least about 90%, at least about 95%) sequence identity to SEQ ID NOs: 148 and 149; SEQ ID NOs: 172 and 173; SEQ ID NOs: 140 and 141; SEQ ID NOs: 174 and 175; SEQ ID NOs: 144 and 145; SEQ ID NOs: 152 and 153; SEQ ID NOs: 146 and 147; SEQ ID NOs: 166 and 167; SEQ ID NOs: 150 and 151; SEQ ID NOs: 142 and 143; SEQ ID NOs: 134 and 135; SEQ ID NOs: 154 and 155; SEQ ID NOs: 136 and 137; and SEQ ID NOs: 138 and 139. In various embodiments, the antigen-binding protein comprises a pair of sequences: one sequence of Table B and another sequence which is a variant sequence having at least or about 70% (e.g., at least about 80%, at least about 85%, at least about 90%, at least about 95%) sequence identity to any of SEQ ID NOs: 134-155 and 166-167 and 172-175. In various embodiments, the antigen-binding protein comprises a pair of sequences: one sequence selected from SEQ ID NOs: 148 and 149; SEQ ID NOs: 172 and 173; SEQ ID NOs: 140 and 141; SEQ ID NOs: 174 and 175; SEQ ID NOs: 144 and 145; SEQ ID NOs: 152 and 153; SEQ ID NOs: 146 and 147; SEQ ID NOs: 166 and 167; SEQ ID NOs: 150 and 151; SEQ ID NOs: 142 and 143; SEQ ID NOs: 134 and 135; SEQ ID NOs: 154 and 155; SEQ ID NOs: 136 and 137; and SEQ ID NOs: 138 and 139, and another sequence which is a variant sequence having at least or about 70% (e.g., at least about 80%, at least about 85%, at least about 90%, at least about 95%) sequence identity to a sequence above. For instance, in various aspects, the antigen-binding protein comprises a sequences of SEQ ID NO: 134 and the antigen-binding protein further comprises a variant sequence having at least or about 70% (e.g., at least about 80%, at least about 85%, at least about 90%, at least about 95%) sequence identity to SEQ ID NO 135.

In various instances, the antigen-binding protein comprises an amino acid sequence of an above-referenced amino acid sequence with one or more amino acid substitutions to reduce or eliminate reactive amino acids to decrease or prevent unwanted side chain reactions. For instance, the antigen-binding protein comprises an amino acid sequence of an above-referenced amino acid sequence with one or more (i) Trp residues substituted with His, Tyr, or Phe; (ii) Asn residues substituted with Gln, Ser, Ala, or Asp; (iii) Asp residues occurring immediately before a Pro residue substituted with Ala, Ser, or Glu, (iv) Asn residues substituted with Gln, Ser, or Ala; and/or (v) Cys residues substituted with Tyr, Ser, or Ala. In various aspects, the antigen-binding protein comprises an amino acid sequence of an above-referenced amino acid sequence with an amino acid substitution predicted to have greater binding affinity, greater stability, or other positive attribute, based on SHM events or based on statistical analyses of a multitude of other similar antibody sequences. In some aspects, the antigen-binding protein comprises (a) an HC CDR1 amino acid sequence set forth in Table A1 or a sequence selected from the group consisting of: SEQ ID NOs: 452, 455, 461, 465, and 71, or a variant sequence thereof which differs by only one or two amino acids or which has at least or about 70% (e.g., at least about 80%, at least about 85%, at least about 90%, at least about 95%) sequence identity; (b) an HC CDR2 amino acid sequence set forth in Table A1 or a sequence selected from the group consisting of: SEQ ID NOs: 475, 456, 462, 466, and 468; or a variant sequence thereof which differs by only one or two amino acids or which has at least or about 70% (e.g., at least about 80%, at least about 85%, at least about 90%, at least about 95%) sequence identity; (c) an HC CDR3 amino acid sequence set forth in Table A1 or a sequence selected from the group consisting of: SEQ ID NOs: 453, 457, 463, 467, and 469; or a variant sequence thereof which differs by only one or two amino acids or which has at least or about 70% (e.g., at least about 80%, at least about 85%, at least about 90%, at least about 95%) sequence identity; (d) a LC CDR1 amino acid sequence set forth in Table A1 or a sequence selected from the group consisting of: SEQ ID NOs: 449, 476, 458, 464, and 68; or a variant sequence thereof which differs by only one or two amino acids or which has at least or about 70% (e.g., at least about 80%, at least about 85%, at least about 90%, at least about 95%) sequence identity; (e) an LC CDR2 amino acid sequence set forth in Table A1 or a sequence selected from the group consisting of: SEQ ID NOs: 450, 477, 459, 57, and 69; or a variant sequence thereof which differs by only one or two amino acids or which has at least or about 70% (e.g., at least about 80%, at least about 85%, at least about 90%, at least about 95%) sequence identity; (f) an LC CDR3 amino acid sequence set forth in Table A1 or a sequence selected from the group consisting of: SEQ ID NOs: 451, 454, 460, 58, and 70, or a variant sequence thereof which differs by only one or two amino acids or which has at least or about 70% (e.g., at least about 80%, at least about 85%, at least about 90%, at least about 95%) sequence identity or (g) a combination of any two or more of (a)-(f).

TABLE A1

| | LC CDR1 | LC CDR2 | LC CDR3 | HC CDR1 | HC CDR2 | HC CDR3 |
|---|---|---|---|---|---|---|
| AB1* | 449 | 450 | 451 | 452 | 475 | 453 |
| AB3* | 476 | 477 | 454 | 455 | 456 | 457 |
| AB4* | 458 | 459 | 460 | 461 | 462 | 463 |
| AB9* | 464 | 57 | 58 | 465 | 466 | 467 |
| AB11* | 68 | 69 | 70 | 71 | 468 | 469 |

In some aspects, the HC CDR1 comprises Gly immediately N-terminal of SEQ ID NO: 452 and, optionally, in some aspects, the HC CDR1 comprises MX immediately C-terminal of SEQ ID NO: 452, wherein X is H, N, or S. In various aspects, the HC CDR3 comprises Ala immediately N-terminal of SEQ ID NO: 453. In various aspects, the LC CDR1 further comprises TAS immediately N-terminal of SEQ ID NO: 449, and, optionally, XH immediately C-terminal of SEQ ID NO: 449, wherein X is H, S, Y, or Q. In some aspects, as described below, the first amino acid of SEQ ID NO: 449 is S or Q. In some aspects, as described below, the first amino acid of SEQ ID NO: 451 is S or Q.

In various aspects, the HC CDR1 comprises Gly immediately N-terminal of SEQ ID NO: 455, and optionally, in various aspects, the HC CDR1 comprises MX immediately C-terminal of SEQ ID NO: 455, wherein X is N, S, or H. In some aspects, HC CDR2 comprises Gln immediately N-terminal of SEQ ID NO: SEQ ID NO: 456, and optionally H immediately C-terminal of SEQ ID NO: 456. In various aspects, the LC CDR1 comprises RIS immediately N-terminal of SEQ ID NO: 476, and optionally, comprises LA immediately C-terminal of SEQ ID NO: 476. In various aspects, the LC CDR2 comprises XLVE immediately C-terminal of SEQ ID NO: 477, wherein X is I or S.

In various aspects, the HC CDR1 comprises MH immediately C-terminal of SEQ ID NO: 461. In various aspects, the HC CDR2 comprises Tyr immediately N-terminal of SEQ ID NO: 462, and optionally, TH immediately C-terminal of SEQ ID NO: 462. In exemplary aspects, the HC CDR3 does not include the first two amino acids of SEQ ID NO: 463. In various aspects, the LC CDR1 comprises RSS immediately N-terminal of SEQ ID NO: 458, and optionally, LN immediately C-terminal of SEQ ID NO: 458. In various aspects, the LC CDR2 comprises XRFS immediately C-terminal of SEQ ID NO: 459, wherein X is Q, S, A, or D.

In various aspects, the HC CDR1 comprises MH immediately C-terminal of SEQ ID NO: 465. In various aspects, the HC CDR2 comprises YI immediately N-terminal of SEQ ID NO: 466, and optionally, Xaa immediately C-terminal of SEQ ID NO: 466, wherein Xaa is N, S, Q, or A. In various aspects, the LC CDR1 comprises LAS immediately N-terminal of SEQ ID NO: 464, and optionally, LA immediately C-terminal of SEQ ID NO: 464. In various aspects, the LC CDR2 comprises SLAD immediately C-terminal of SEQ ID NO: 57.

In various aspects, the HC CDR1 comprises MH immediately C-terminal of SEQ ID NO: 71. In various aspects, the HC CDR2 comprises Tyr immediately N-terminal of SEQ ID NO: 468 and optionally IY immediately C-terminal of SEQ ID NO: 468. In various aspects, the LC CDR1 comprises RAS immediately N-terminal of SEQ ID NO: 68, and optionally SYIH immediately C-terminal to SEQ 68. In various aspects, the LC CDR2 comprises XLES immediately C-terminal to SEQ ID NO: 69, wherein X is N, Q, S, A, or D.

In various aspects, the antigen-binding protein comprises a LC CDR1 amino acid sequence, a LC CDR2 amino acid sequence, and a LC CDR3 amino acid sequence set forth in Table A1 and at least 1 or 2 of the HC CDR amino acid sequences set forth in Table A1. In various aspects, the antigen-binding protein comprises a HC CDR1 amino acid sequence, a HC CDR2 amino acid sequence, and a HC CDR3 amino acid sequence set forth in Table A1 and at least 1 or 2 of the LC CDR amino acid sequences set forth in Table A1.

In various embodiments, the antigen-binding protein comprises at least 3, 4, or 5 of the amino acid sequences designated by the SEQ ID NOs: in a single row of Table A1. In various embodiments, the antigen-binding protein comprises each of the LC CDR amino acid sequences designated by the SEQ ID NOs: of a single row of Table A1 and at least 1 or 2 of the HC CDR amino acid sequences designated by the SEQ ID NOs: in of a single row of Table A1. In various embodiments, the antigen-binding protein comprises each of the HC CDR amino acid sequences designated by the SEQ ID NOs: of a single row of Table A1 and at least 1 or 2 of the LC CDR amino acid sequences designated by the SEQ ID NOs: of a single row of Table A1. In various embodiments, the antigen-binding protein comprises all 6 of the CDR amino acid sequences designated by the SEQ ID NOs: of a single row of Table A1. In various embodiments, the antigen-binding protein comprises six CDR amino acid sequences selected from the group consisting of: (a) SEQ ID NOs: 449-453 and 475; (b) SEQ ID NOs: 476-477, 454-457; (c) SEQ ID NOs: 458-463; (d) SEQ ID NOs: 57, 58, 464-467; and (e) SEQ ID NOs: 68-71 and 468-469.

In various instances, the amino acid sequences of Table A1 are separated by at least one or more (e.g., at least 2, 3, 4, 5, 6, 7, 8, 9, 10, or more) intervening amino acid(s). In various instances, there are about 10 to about 20 amino acids between the sequences of the LC CDR1 and the LC CDR2 and about 25 to about 40 amino acids between the sequences of the LC CDR2 and the LC CDR3. In various instances, there are about 14 to about 16 amino acids between the sequences of the LC CDR1 and the LC CDR2 and about 30 to about 35 amino acids between the sequences of LC CDR2 and the LC CDR3. In various instances, there are about 10 to about 20 amino acids between the sequences of the HC CDR1 and HC CDR2 and about 25 to about 40 amino acids between the sequences of the HC CDR2 and the HC CDR3. In various instances, there are about 14 to about 16 amino acids between the sequences of the HC CDR1 and HC CDR2 and about 30 to about 35 amino acids between the sequences of the HC CDR2 and HC CDR3.

In various embodiments, the antigen-binding protein comprises (a) a heavy chain variable region amino acid sequence set forth in in Table B1 or a sequence selected from the group consisting of: SEQ ID NO: 478, 480, 482, 486, and 488, or a variant sequence thereof which differs by only one or two amino acids or which has at least or about 70% (e.g., at least about 80%, at least about 85%, at least about 90%, at least about 95%) sequence identity; or (b) a light chain variable region amino acid sequence set forth in Table B1 or a sequence selected from the group consisting of: SEQ ID NO: 479, 481, 483, 487, and 489, or a variant sequence thereof which differs by only one or two amino acids or which has at least or about 70% (e.g., at least about 80%, at least about 85%, at least about 90%, at least about 95%) sequence identity; or (c) both (a) and (b).

TABLE B1

| | HC variable | LC variable |
|---|---|---|
| AB1* | 478 | 479 |
| AB3* | 480 | 481 |
| AB4* | 482 | 483 |
| AB9* | 488 | 489 |
| AB11* | 486 | 487 |

In various embodiments, the antigen-binding protein comprises a pair of amino acid sequences selected from the group consisting of: (a) SEQ ID NO: 478 and 479; (b) SEQ ID NO: 480 and 481; (c) SEQ ID NO: 482 and 483; (d) SEQ ID NO: 486 and 487; and (e) SEQ ID NO: 488 and 489. In various aspects, the antigen-binding protein comprises a variant sequence of a sequence having a SEQ ID NO: listed in Table B1 which differs by only one or two amino acids or which has at least or about 70% (e.g., at least about 80%, at least about 85%, at least about 90%, at least about 95%) sequence identity, wherein the different amino acid(s) occur(s) at the positions described below in “Humanized Antibodies”.

### Humanized Antibodies

In various aspects, the antigen-binding protein is a humanized version of an antigen binding protein described in Table A, Table A1, Table B, or Table B1.

### Humanized AB1

In various aspects, the antigen-binding protein is a humanized version of AB1 as set forth in Table B or B1 with one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35) amino acid substitutions in the heavy chain variable region at one or more of the following positions: 5, 8, 11, 12, 13, 20, 31, 33, 35, 38, 40, 48, 50, 55, 57, 59, 61, 65, 66, 67, 68, 70, 72, 74, 76, 79, 80, 82, 87, 90, 91, 98, 101, and 116. In various instances, the antigen-binding protein comprises an amino acid sequence of SEQ ID NO: 428. In various aspects, the antigen-binding protein is a humanized version of AB1 as set forth in Table B or B1 with one or more amino acid substitutions in the heavy chain variable region at one or more (e.g., 1, 2, 3, 4, 5, 6, 7, 8, 9, 10, 11, or 12) of the following positions: 20, 31, 35, 48, 50, 59, 67, 70, 74, 79, 98, 101. In various instances, the antigen-binding protein comprises an amino acid sequence of SEQ ID NO: 429. In various aspects, the amino acids at the above-recited positions are selected from the amino acids according to the table below:

| Position | Amino acids | Position | Amino acids | Position | Amino acids |
|---|---|---|---|---|---|
| 5 | Q, V | 8 | A, G | 11 | L, V |
| 12 | A, K | 13 | R, K | 20 | M, V |
| 31 | S, T, V, D | 33 | Y, T | 35 | H, N, S |
| 38 | K, R | 40 | R, A | 48 | I, M |
| 50 | F, V, T, Y, I | 55 | G, S | 57 | S, Y |
| 59 | D, E, N, S | 61 | N, A | 65 | K, Q |
| 66 | D, G | 67 | R, Q, N, K | 68 | T, V |
| 70 | L, M | 72 | R, A | 74 | K, T |
| | | 79 | V, D, S, A | 82 | Q, E |
| 87 | T, R | 91 | S, T | 98 | N, Q, H, D, R |
| 101 | Y | 76 | ST | 116 | A, S |

In various aspects, the antigen-binding protein is a humanized version of AB1 as set forth in Table B or B1 with one or more amino acid substitutions in the light chain variable region at one or more e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41) of the following positions: 1, 3, 4, 9, 10, 11, 15, 17, 21, 24, 27, 29, 32, 34, 35, 43, 44, 48, 51, 52, 53, 54, 55, 56, 61, 67, 71, 72, 73, 79, 80, 81, 84, 90, 92, 93, 94, 95, 96, 101, 107. In various instances, the antigen-binding protein comprises an amino acid sequence of SEQ ID NO: 430. In various aspects, the antigen-binding protein is a humanized version of AB1 as set forth in Table B or B1 with one or more amino acid substitutions in the light chain variable region at one or more (e.g., 1, 2, 3, 4, 5, 6, 7, 8, 9, 11, 12, or 13) of the following positions: 4, 21, 32, 34, 48, 51, 53, 61, 67, 79, 84, 91, and 93. In various instances, the antigen-binding protein comprises an amino acid sequence of SEQ ID NO: 431. In various aspects, the amino acids at the above-recited positions are selected from the amino acids according to the table below:

| Position | Amino acids | Position | Amino acids | Position | Amino acids |
|---|---|---|---|---|---|
| 1 | Q, D | 3 | V, Q | 4 | L, M |
| 9 | A, S | 10 | I, S | 11 | M, L |
| 15 | L, V | 17 | E, D | 21 | M, I |
| 24 | T, R | 27 | S, Q | 32 | T, V, F, D, S |
| 34 | F, L | 35 | H, S, Y, Q, N | 43 | S, K |
| 44 | S, A | 48 | W, L | 51 | S, T, Q, A |
| 52 | T, A | 53 | S, T, D, Q | 54 | N, S |
| | | 56 | A, Q | 61 | R, Q, S, D, |
| 67 | A, S, T, G | 71 | S, D | 72 | Y, F |
| 73 | S, T | 79 | M, L | 80 | E, Q |
| 81 | A, P | 84 | A, F | 90 | H, Q |
| 91 | Q, H, S | 93 | H, Q, S, Y | 94 | R, S |
| 97 | L, P | 101 | A, Q | 107 | L, I |
| 29 | V, I | 92 | Y, S | 95 | S, T |

### Humanized AB3

In various aspects, the antigen-binding protein is a humanized version of AB3 as set forth in Table B or B1 with one or more amino acid substitutions in the heavy chain variable region at one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33) of the following positions: 3, 5, 18, 19, 23, 31, 33, 35, 40, 42, 49, 50, 52, 53, 54, 55, 56, 57, 58, 59, 61, 64, 76, 79, 80, 81, 87, 94, 95, 99, 106, 112, 114. In various instances, the antigen-binding protein comprises an amino acid sequence of SEQ ID NO: 432. In various aspects, the antigen-binding protein is a humanized version of AB3 as set forth in Table B or B1 with one or more amino acid substitutions in the heavy chain variable region at one or more (e.g., 1, 2, 3, 4, 5, 6, or 7) of the following positions: 31, 35, 50, 55, 79, 99, 106. In various instances, the antigen-binding protein comprises an amino acid sequence of SEQ ID NO: 433. In various aspects, the amino acids at the above-recited positions are selected from the amino acids according to the table below:

| Position | Amino acids | Position | Amino acids | Position | Amino acids |
|---|---|---|---|---|---|
| 3 | K, Q | 5 | E, L | 18 | M, L |
| 19 | K, R | 23 | V, A | 31 | N, S, R |
| 33 | W, A | 35 | N, S, H | 40 | S, A |
| 42 | E, G | 49 | A, S | 50 | Q, S, N, |
| | | | | | H, A |
| | | 52 | R, S | 53 | L, G |
| 54 | K, S | 55 | S, N, T, | 56 | D, G |
| | | | A, G | 59 | A, S |
| 61 | H, Y | 64 | E, D | 76 | D, N |
| 79 | R, N, Q, D, S | 80 | S, T | 81 | V, L |
| 87 | N, S | 94 | G, A | 95 | T, V, I |
| 99 | N, D, T, K, A | 106 | C, Y, A, | 112 | T, L |
| 114 | I, T | | S, T | | |

In various aspects, the antigen-binding protein is a humanized version of AB3 as set forth in Table B or B1 with one or more amino acid substitutions in the light chain variable region at one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or 29) of the following positions: 9, 17, 18, 25, 27, 28, 30, 34, 40, 43, 45, 48, 50, 52, 53, 55, 56, 70, 72, 74, 76, 84, 85, 90, 91, 93, 94, 97, and 100. In various instances, the antigen-binding protein comprises an amino acid sequence of SEQ ID NO: 434. In various aspects, the antigen-binding protein is a humanized version of AB3 as set forth in Table B or B1 with one or more amino acid substitutions in the light chain variable region at one or more (e.g., 1, 2, 3, 4, 5, 6, 7, 8, or 9) of the following positions: 25, 34, 48, 53, 55, 84, 85, 90, and 93. In various instances, the antigen-binding protein comprises an amino acid sequence of SEQ ID NO: 435. In various aspects, the amino acids at the above-recited positions are selected from the amino acids according to the table below:

| Position | Amino acids | Position | Amino acids | Position | Amino acids |
|---|---|---|---|---|---|
| 9 | A, S | 17 | E, D | 18 | T, R |
| 25 | I, V, L, T, A | 34 | A, S, N | 40 | Q, P |
| 43 | S, A | 45 | Q, K | 48 | V, I |
| 53 | I, V, L, T, S | 55 | V, T, L, A, Q | 70 | Q, D |
| 72 | S, T | 74 | K, T | 76 | N, S |
| 84 | G, A | 85 | N, Q, S, T | 90 | H, Q, S, T |
| 93 | T, S, N, G | 100 | G, Q | 27 | E, Q |
| 28 | N, S | 30 | Y, S | 50 | N, A |
| 52 | K, S | 56 | E, S | 91 | H, S |
| 94 | V, T | 97 | T, P | | |

### Humanized AB4

In various aspects, the antigen-binding protein is a humanized version of AB4 as set forth in Table B or B1 with one or more amino acid substitutions in the heavy chain variable region at one or more (e.g., 1, 2, 3, 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, or 36 of the following positions: 5, 11, 12, 13, 20, 29, 31, 33, 37, 38, 40, 45, 48, 50, 55, 56, 57, 59, 61, 62, 65, 66, 67, 68, 70, 72, 74, 76, 79, 82, 84, 87, 91, 97, 101, 117. In various instances, the antigen-binding protein comprises an amino acid sequence of SEQ ID NO: 436. In various aspects, the antigen-binding protein is a humanized version of AB4 as set forth in Table B or B1 with one or more amino acid substitutions in the heavy chain variable region at one or more (e.g., 1, 2, 3, 4, 5, 6, 7, 8, 9, 10, 11, 12, 13, 14, 15, 16, 17, 18, or 19) of the following positions: 20, 29, 31, 37, 45, 48, 56, 59, 61, 62, 65, 66, 68, 70, 74, 79, 84, 97, and 101. In various instances, the antigen-binding protein comprises an amino acid sequence of SEQ ID NO: 437. In various aspects, the amino acids at the above-recited positions are selected from the amino acids according to the table below:

| Position | Amino acids | Position | Amino acids | Position | Amino acids |
|---|---|---|---|---|---|
| 5 | Q, V | 11 | L, V | 12 | A, K |
| 13 | R, K | 20 | M, V | 33 | T, Y |
| 37 | I, V, F, Y | 38 | K, R | 40 | R, A |
| 45 | Q, L, V, T, N | 48 | I, M | 50 | Y, I |
| 55 | S, G | 56 | T, G, S, V, | 57 | Y, S |
| | | | D | | |
| 59 | H, K, S, Q, N | 61 | I, A, N, F, | 62 | K, Q |
| | | | Y, V | | |
| 65 | K, Q | 66 | D, G | 67 | K, R |
| 68 | A, V | 70 | L, M | 72 | A, R |
| 74 | T, K | 76 | S, T | 79 | A, V |
| 82 | Q, E | 84 | R, S, Q, D | 87 | T, R |
| 91 | S, T | 97 | S, A, T, V | 101 | L, V, F |
| 117 | A, S | 29 | F, Y, S, T | 31 | S, T, Y, D |

In various aspects, the antigen-binding protein is a humanized version of AB4 as set forth in Table B or B1 with one or more amino acid substitutions in the light chain variable region at one or more (e.g., 1, 2, 3, 4, 5, 6, 7, 8, 9, 10, 11, 12, 13, 14, 15, 16, 17, 18, 19, 20, 21, 22, 23, or 24) of the following positions: 7, 14, 17, 18, 31, 33, 39, 41, 42, 44, 50, 51, 55, 57, 60, 81, 88, 92, 94, 95, 96, 99, 100, 105. In various instances, the antigen-binding protein comprises an amino acid sequence of SEQ ID NO: 438. In various aspects, the antigen-binding protein is a humanized version of AB4 as set forth in Table B or B1 with one or more amino acid substitutions in the light chain variable region at one or more (e.g., 1, 2, 3, 4, 5, 6, or 7) of the following positions: 33, 39, 55, 57, 81, 95, and 96. In various instances, the antigen-binding protein comprises an amino acid sequence of SEQ ID NO: 439. In various aspects, the amino acids at the above-recited positions are selected from the amino acids according to the table below:

| Position | Amino acids | Position | Amino acids | Position | Amino acids |
|---|---|---|---|---|---|
| 7 | T, S | 14 | S, T | 17 | D, Q |
| 18 | Q, P | 31 | Y, H | 33 | D, N, E, Q |
| 39 | H, N, Q, D | 41 | F, Y | 42 | L, Q |
| 44 | K, R | 50 | K, R | 51 | R, L |
| 55 | K, R, Q | 57 | S, T, V | 60 | D, F |
| 81 | R, S, N, D | 88 | L, V | 92 | F, Y |
| 94 | M, S | 95 | Q, H, T | 96 | S, T, G, D |
| 99 | W, V | 105 | G, Q | | |

### Humanized AB9

In various aspects, the antigen-binding protein is a humanized version of AB9 as set forth in Table B or B1 with one or more amino acid substitutions in the heavy chain variable region at one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or 29) of the following positions: 1, 5, 9, 11, 12, 20, 38, 40, 41, 43, 44, 48, 61, 63, 65, 67, 69, 70, 72, 73, 74, 76, 79, 84, 87, 91, 93, 112, and 113. In various instances, the antigen-binding protein comprises an amino acid sequence of SEQ ID NO: 444. In various aspects, the amino acids at the above-recited positions are selected from the amino acids according to the table below:

| Position | Amino acids | Position | Amino acids | Position | Amino acids |
|---|---|---|---|---|---|
| 1 | E, Q | 5 | Q, V | 9 | P, A |
| 11 | L, V | 12 | V, K | 20 | M, V |
| 38 | K R, | 40 | S, A | 41 | H, P |
| 43 | K Q | 44 | S, G | 48 | I, M |
| 61 | N, A | 63 | N, K | 65 | K, Q |
| 67 | K, R | 69 | A, V | 70 | L, M |
| 72 | V, R | 73 | N, D | 74 | K, T |
| 76 | S, T | 79 | A, V | 84 | R, S |
| 87 | T, R | 91 | S, T | 93 | A, V |
| 112 | T, L | 113 | L, V | | |

In various aspects, the antigen-binding protein is a humanized version of AB9 as set forth in Table B or B1 with one or more amino acid substitutions in the light chain variable region at one or more (e.g., 1, 2, 3, 4, 5, 6, 7, 8, 9, 10, 11, 12, 13, 14, or 15) of the following positions: 9, 11, 15, 17, 18, 43, 45, 70, 72, 73, 74, 80, 84, 85, and 100. In various instances, the antigen-binding protein comprises an amino acid sequence of SEQ ID NO: 445. In various aspects, the amino acids at the above-recited positions are selected from the amino acids according to the table below:

| Position | Amino acids | Position | Amino acids | Position | Amino acids |
|---|---|---|---|---|---|
| 9 | A, S | 11 | Q, L | 15 | L, V |
| 17 | E, D | 18 | S, R | 43 | S, A |
| 45 | Q, K | 70 | R, D | 72 | S, T |
| 73 | F, L | 74 | K, R | 80 | A, P |
| 84 | V, A | 85 | S, T | 100 | G, Q |

### Humanized AB11

In various aspects, the antigen-binding protein is a humanized version of AB11 as set forth in Table B or B1 with one or more amino acid substitutions in the heavy chain variable region at one or more (e.g., 1, 2, 3, 4, 5, 6, 7, 8, 9, 10, 11, 12, 13, 14, or 15) of the following positions: 1, 15, 18, 19, 42, 49, 63, 75, 76, 78, 80, 84, 88, and 93. In various instances, the antigen-binding protein comprises an amino acid sequence of SEQ ID NO: 446. In various aspects, the amino acids at the above-recited positions are selected from the amino acids according to the table below:

| Position | Amino acids | Position | Amino acids | Position | Amino acids |
|---|---|---|---|---|---|
| 1 | D, E | 15 | R, G | 18 | R, L |
| 19 | K, R | 42 | E, G | 49 | A, S |
| 63 | T, S | 75 | P, A | 76 | T, K |
| 78 | T, S | 80 | F, Y | 84 | T, N |
| 88 | S, A | 93 | M, V | | |

In various aspects, the antigen-binding protein is a humanized version of AB11 as set forth in Table B or B1 with one or more amino acid substitutions in the light chain variable region at one or more (e.g., 1, 2, 3, 4, 5, 6, 7, 8, 9, 10, 11, 12, 13, 14, or 15) of the following positions: 4, 9, 17, 22, 64, 78, 80, 81, 82, 83, 84, 87, 89, 104, and 110, optionally, one or more of the following positions: 4, 82, 110. In various instances, the antigen-binding protein comprises an amino acid sequence of SEQ ID NO: 447 or 448. In various aspects, the amino acids at the above-recited positions are selected from the amino acids according to the table below:

| Position | Amino acids | Position | Amino acids | Position | Amino acids |
|---|---|---|---|---|---|
| 4 | L, M | 9 | A, D | 17 | Q, E |
| 22 | S, N | 64 | A, D | 78 | N, T |
| 80 | H, S | 81 | P, S | 82 | V, L |
| 83 | E, Q | 84 | E, A | 87 | A, V |
| 89 | T, V | 104 | A, Q | 110 | L, I |

In various embodiments, the antigen-binding protein comprises (a) a heavy chain variable region amino acid sequence set forth in in Table C or a sequence selected from the group consisting of: 376-379, 384-387, 391-396, 403-408, 412, 413, and 422-427 or a variant sequence thereof which differs by only one or two amino acids or which has at least or about 70%, or about 80%, or about 85%, or about 90%, or about 95% sequence identity; or (b) a light chain variable region amino acid sequence set forth in Table C or a sequence selected from the group consisting of: 380-383, 388-390, 397-402, 409-411, 414, 415, or a variant sequence thereof which differs by only one or two amino acids or which has at least or about 70%, or about 80%, or about 85%, or about 90%, or about 95% sequence identity; or (c) both (a) and (b).

TABLE C

| | Humanized Light | Humanized Heavy |
|---|---|---|
| | Chain Variable Region | Chain Variable Region |
| AB1 | 380, 381, 382, 383 | 376, 377, 378, 379 |
| AB3 | 388, 389, 390 | 384, 385, 386, 387, 422 |
| AB4 | 397, 398, 399, | 391, 392, 392, 394, 395, |
| | 400, 401, 402 | 396, 423, 424, 425, 426, 427 |
| AB9 | 409, 410, 411, | 403, 404, 405, 406, 407, 408 |
| AB11 | 414, 415 | 412, 413 |

In various embodiments, the humanized antigen-binding protein comprises a pair of amino acid sequences as shown in Table D. Further embodiments include CLDN6-specific antigen-binding proteins comprising CDRs1-3 from a heavy chain variable region shown in Table D; and CDRs1-3 from a light chain variable region shown in Table D. In a preferred embodiment, the CLDN6-specific antigen-binding protein comprises (i) CDRs1-3 from a heavy chain variable region comprising the amino acid sequence set forth in SEQ ID NO: 387; and (ii) CDRs1-3 from a light chain variable region comprising the amino acid sequence set forth in SEQ ID NO: 389 of Table D.

TABLE D

| Humanized AB | HC | LC |
|---|---|---|
| 1-1 | 376 | 380 |
| 1-2 | 377 | 380 |
| 1-3 | 377 | 381 |
| 1-4 | 377 | 382 |
| 1-5 | 377 | 383 |
| 1-6 | 378 | 381 |
| 1-7 | 378 | 382 |
| 1-8 | 378 | 383 |
| 1-9 | 379 | 381 |
| 1-10 | 379 | 382 |
| 1-11 | 379 | 383 |
| 3-1 | 384 | 388 |
| 3-2 | 385 | 388 |
| 3-3 | 385 | 389 |
| 3-4 | 386 | 388 |
| 3-5 | 386 | 389 |
| 3-6 | 387 | 388 |
| 3-7 | 387 | 389 |
| 3-9 | 422 | 389 |
| 4-1 | 391 | 397 |
| 4-2 | 392 | 397 |
| 4-3 | 392 | 398 |
| 4-4 | 393 | 398 |
| 4-5 | 394 | 398 |
| 4-6 | 395 | 398 |
| 4-7 | 396 | 398 |
| 4-8 | 423 | 398 |
| 4-9 | 424 | 398 |
| 4-10 | 425 | 398 |
| 4-11 | 426 | 398 |
| 4-12 | 427 | 398 |
| 9-1 | 403 | 409 |
| 9-2 | 404 | 409 |
| 9-3 | 405 | 410 |
| 9-4 | 405 | 411 |
| 9-5 | 406 | 410 |
| 9-6 | 406 | 411 |
| 9-7 | 407 | 410 |
| 9-8 | 407 | 411 |
| 9-9 | 408 | 410 |
| 9-10 | 408 | 411 |
| 11-1 | 412 | 414 |
| 11-2 | 413 | 414 |
| 11-3 | 413 | 415 |

In various embodiments, the antigen-binding protein comprises a pair of variant sequences, each having at least or about 70% (e.g., at least about 80%, at least about 85%, at least about 90%, at least about 95%) sequence identity to a SEQ ID NO listed in Table C. In various embodiments, the antigen-binding protein comprises a pair of sequences: one sequence selected from a SEQ ID NO: listed in Table C and another sequence which is a variant sequence having at least or about 70% (e.g., at least about 80%, at least about 85%, at least about 90%, at least about 95%) sequence identity to a sequence having a SEQ ID NO: listed in Table D a sequence having a SEQ ID NO: listed in Table C.

In various embodiments, the antigen-binding protein comprises a pair of sequences: one sequence selected from a SEQ ID NO listed in Table D, and another sequence which is a variant sequence having at least or about 70% (e.g., at least about 80%, at least about 85%, at least about 90%, at least about 95%) sequence identity to a sequence having a SEQ ID NO listed in Table D. For instance, in various aspects, the antigen-binding protein comprises a sequences of SEQ ID NO: 387 and the antigen-binding protein further comprises a variant sequence having at least or about 70% (e.g., at least about 80%, at least about 85%, at least about 90%, at least about 95%) sequence identity to SEQ ID NO 389.

In various instances, the antigen-binding protein is a humanized antigen-binding protein as set forth in Table D with one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35) amino acid substitutions in the heavy chain (HC) variable region or in the light chain (LC) variable region, or in both. In exemplary aspects, the antigen-binding protein is a humanized antigen-binding protein of AB1-11 with one or more amino acid substitutions in the HC variable region, the LC variable region, or both. In exemplary aspects, the antigen-binding protein comprises a HC of SEQ ID NO: 379 with 1, 2, 3, 4, or 5 amino acid substitutions. In exemplary aspects, the antigen-binding protein comprises a HC CDR1 of SEQ ID NO: 504, a HC CDR2 of SEQ ID NO: 505, a HC CDR3 of SEQ ID NO: 506, or a combination thereof. In exemplary instances, the antigen-binding protein comprises a HC of SEQ ID NO: 503. In some aspects, the antigen-binding protein comprises a HC of any one of SEQ ID NOs: 496-501. In various instances, the light chain variable region comprises a LC CDR1 of SEQ ID NO: 449, a LC CDR2 of SEQ ID NO: 450, a LC CDR3 of SEQ ID NO: 451, or a combination thereof. In some aspects, the antigen-binding protein comprises a LC of any one of SEQ ID NOs: 380-383, and 479. In exemplary instances, the antigen-binding protein comprises a LC of SEQ ID NO: 383. In exemplary aspects, the antigen-binding protein is a humanized antigen-binding protein of AB3-7 with one or more amino acid substitutions in the HC variable region, the LC variable region, or both. In exemplary aspects, the antigen-binding protein comprises a HC of SEQ ID NO: 387 with 1, 2, 3, 4, 5, or 6 amino acid substitutions. In exemplary aspects, the antigen-binding protein comprises a HC CDR1 of SEQ ID NO: 507, a HC CDR2 of SEQ ID NO: 508, a HC CDR3 of SEQ ID NO: 509, or a combination thereof. In exemplary instances, the antigen-binding protein comprises a HC of SEQ ID NO: 502. In some aspects, the antigen-binding protein comprises a HC of any one of SEQ ID NOs: 490-495. In various instances, the light chain variable region comprises a LC CDR1 of SEQ ID NO: 476, a LC CDR2 of SEQ ID NO: 477, a LC CDR3 of SEQ ID NO: 454, or a combination thereof. In some aspects, the antigen-binding protein comprises a LC of any one of SEQ ID NOs: 388-390, and 481. In exemplary instances, the antigen-binding protein comprises a LC of SEQ ID NO: 389. In exemplary aspects, the antigen-binding protein is a humanized antigen-binding protein of AB3 with one or more amino acid substitutions in the HC variable region, the LC variable region, or both. In exemplary aspects, the antigen-binding protein comprises a HC of SEQ ID NO: 139 with 1, 2, 3, 4, or 5 (or more) amino acid substitutions. In some aspects, the antigen-binding protein comprises a HC of any one of SEQ ID NOs: 510 In exemplary aspects, the antigen-binding protein comprises a HC of SEQ ID NO: 138 with 1, 2, 3, 4, or 5 (or more) amino acid substitutions. In some aspects, the antigen-binding protein comprises a HC of any one of SEQ ID NOs: 511. In exemplary aspects, the antigen-binding protein is a humanized antigen-binding protein of AB1 with one or more amino acid substitutions in the HC variable region, the LC variable region, or both. In exemplary aspects, the antigen-binding protein comprises a HC of SEQ ID NO: 135 with 1, 2, 3, 4, or 5 (or more) amino acid substitutions. In some aspects, the antigen-binding protein comprises a HC of any one of SEQ ID NOs: 513. In exemplary aspects, the antigen-binding protein comprises a HC of SEQ ID NO: 134 with 1, 2, 3, 4, or 5 (or more) amino acid substitutions. In some aspects, the antigen-binding protein comprises a HC of any one of SEQ ID NOs: 512. In some aspects, the antigen-binding protein comprises a HC sequence of SEQ ID NO: 512 with 1, 2, 3, 4, or 5 (or more) amino acid substitutions.

### Compositions, Pharmaceutical Compositions and Formulations

Compositions comprising an antigen-binding protein as presently disclosed are provided herein.

In some aspects, the composition comprises agents which enhance the chemico-physico features of the antigen-binding protein, e.g., via stabilizing the antigen-binding protein at certain temperatures, e.g., room temperature, increasing shelf life, reducing degradation, e.g., oxidation protease mediated degradation, increasing half-life of the antigen-binding protein, etc. In some aspects, the composition comprises any of the agents disclosed herein as a heterologous moiety, optionally in admixture with the antigen-binding proteins of the present disclosure or conjugated to the antigen-binding proteins.

In various aspects of the present disclosure, the composition additionally comprises a pharmaceutically acceptable carrier, diluents, or excipient. In some embodiments, the conjugate as presently disclosed (hereinafter referred to as “active agent”) is formulated into a pharmaceutical composition comprising the active agent, along with a pharmaceutically acceptable carrier, diluent, or excipient. In this regard, the present disclosure further provides pharmaceutical compositions comprising an active agent which is intended for administration to a subject, e.g., a mammal.

In some embodiments, the active agent is present in the pharmaceutical composition at a purity level suitable for administration to a patient. In some embodiments, the active agent has a purity level of at least about 90%, about 91%, about 92%, about 93%, about 94%, about 95%, about 96%, about 97%, about 98% or about 99%, and a pharmaceutically acceptable diluent, carrier or excipient. In some embodiments, the compositions contain an active agent at a concentration of about 0.001 to about 30.0 mg/ml.

In various aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In various aspects, the pharmaceutical composition comprises formulation materials that are nontoxic to recipients at the dosages and concentrations employed. In specific embodiments, pharmaceutical compositions comprising an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In various aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, glutamate or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20 or polysorbate 80 (PS80), triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18” Edition, (A. R. Genrmo, ed.), 1990, Mack Publishing Company.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In various embodiments, the pH of the pharmaceutical composition is between 5.5 and 7.5.

### Routes of Administration

With regard to the present disclosure, the active agent, or pharmaceutical composition comprising the same, can be administered to the subject via any suitable route of administration. For example, the active agent can be administered to a subject via parenteral administration, such as, for example, intravenous infusion or subcutaneous injection.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The active agent of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight of the active agent of the present disclosure in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed., pages 622-630 (1986)). Injectable formulations suitable for subcutaneous administration may be co-formulated with hyaluronidase (U.S. Pat. No. 9,084,743).

### Kits

In some embodiments, the antigen-binding proteins of the present disclosure are provided in a kit. In various aspects, the kit comprises the ADC as a unit dose. For purposes herein “unit dose” refers to a discrete amount dispersed in a suitable carrier. In various aspects, the unit dose is the amount sufficient to provide a subject with a desired effect, e.g., inhibition of tumor growth, reduction of tumor size, treatment of cancer. Accordingly, provided herein are kits comprising an ADC of the present disclosure optionally provided in unit doses. In various aspects, the kit comprises several unit doses, e.g., a week or month supply of unit doses, optionally, each of which is individually packaged or otherwise separated from other unit doses. In some embodiments, the components of the kit/unit dose are packaged with instructions for administration to a patient. In some embodiments, the kit comprises one or more devices for administration to a patient, e.g., a needle and syringe, and the like. In some aspects, the ADC of the present disclosure is pre-packaged in a ready to use form, e.g., a syringe, an intravenous bag, etc. In some aspects, the kit further comprises other therapeutic or diagnostic agents or pharmaceutically acceptable carriers (e.g., solvents, buffers, diluents, etc.), including any of those described herein.

### Various Embodiments

In certain embodiments, the disclosure relates to a method for inhibiting a solid tumor expressing claudin-6 in a human subject, comprising administering to the human subject an effective amount of a composition comprising conjugates of a CLDN6-specific antigen-binding protein covalently bound to heterologous moieties comprising structural formula (I):

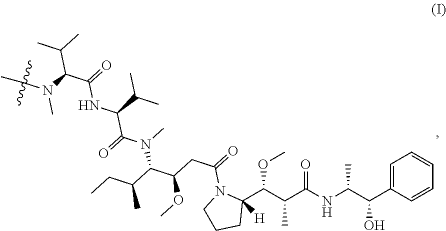 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein
a first plurality of the conjugates are bound to four heterologous moieties comprising structural formula (I);
at least about 95% of the first plurality of conjugates are structurally homogenous; and
the effective amount is in a range of about
   1.7 mg/kg to 6 mg/kg;
   2.0 mg/kg to 6 mg/kg; or
   2.4 mg/kg to 6 mg/kg.

In certain embodiments, the disclosure relates to any of the methods disclosed herein, wherein at a Cₘₐₓ after administration, the percent of unbound circulating MMAE in serum is less than about 0.01% (w/v) thereby reducing toxicity in the subject and inhibiting the solid tumor. In certain embodiments, the percent of free MMAE is less than about 0.009% at Cₘₐₓ. In certain embodiments, the percent of free MMAE is less than about 0.008% at Cₘₐₓ. In certain embodiments, the percent of free MMAE is less than about 0.007% at Cₘₐₓ. In certain embodiments, the percent of free MMAE is less than about 0.006% at Cₘₐₓ. In certain embodiments, the percent of free MMAE is less than about 0.005% at Cₘₐₓ. In certain embodiments, the percent of free MMAE is less than about 0.004% at Cₘₐₓ. In certain embodiments, the percent of free MMAE is less than about 0.003% at Cₘₐₓ. In certain embodiments, the percent of free MMAE is less than about 0.0000010% at Cₘₐₓ. In certain embodiments, the percent of free MMAE is less than about 0.0000009% at Cₘₐₓ. In certain embodiments, the percent of free MMAE is less than about 0.0000008% at Cₘₐₓ. In certain embodiments, the percent of free MMAE is less than about 0.0000007% at Cₘₐₓ. In certain embodiments, the percent of free MMAE is less than about 0.0000006% at Cₘₐₓ. In certain embodiments, the percent of free MMAE is less than about 0.0000005% at Cₘₐₓ. In certain embodiments, the percent of free MMAE is less than about 0.0000004% at Cₘₐₓ. In certain embodiments, the percent of free MMAE is less than about 0.0000003% at Cₘₐₓ. In certain embodiments, the Cₘₐₓ of the percent of free MMAE is determined after a single dose in cycle 1. In a separate embodiment, the Cₘₐₓ of the percent of free MMAE is determined in cycle 3 after three separate administrations, three weeks apart of the composition comprising the conjugates of a CLDN6-specific antigen-binding protein covalently bound to heterologous moieties. In certain embodiments, the conjugate is an anti-CLDN6-specific antibody conjugated to a chemotherapeutic or cytotoxic agent. In certain embodiments, the conjugate is TORL-1-23.

In certain embodiments, the disclosure relates to any of the methods disclosed herein, wherein at a Cₘₐₓ after administration, the unbound circulating MMAE in serum is less than about 5 ng/mL, thereby reducing toxicity in the subject and inhibiting the solid tumor. In certain embodiments, the free MMAE is less than about 4 ng/mL at Cₘₐₓ. In certain embodiments, the free MMAE is less than about 3 ng/mL at Cₘₐₓ. In certain embodiments, the free MMAE is less than about 2 ng/mL at Cₘₐₓ. In certain embodiments, the free MMAE is less than about 1 ng/mL at Cₘₐₓ. In certain embodiments, the free MMAE is less than about 0.5 ng/mL at Cₘₐₓ. In certain embodiments, the free MMAE is less than about 0.15 ng/mL at Cₘₐₓ. In certain embodiments, the free MMAE is less than about 0.12 ng/mL at Cₘₐₓ.

In certain embodiments, the disclosure relates to any of the methods disclosed herein, wherein at a Cₘₐₓ after administration, the unbound circulating MMAE in serum is less than about 10 ng/mL, thereby reducing toxicity in the subject and inhibiting the solid tumor. In certain embodiments, the free MMAE is less than about 9 ng/mL at Cₘₐₓ. In certain embodiments, the free MMAE is less than about 8 ng/mL at Cₘₐₓ. In certain embodiments, the free MMAE is less than about 7 ng/mL at Cₘₐₓ. In certain embodiments, the free MMAE is less than about 6 ng/mL at Cₘₐₓ. In certain embodiments, the free MMAE is less than about 5 ng/mL at Cₘₐₓ. In certain embodiments, the free MMAE is less than about 4 ng/mL at Cₘₐₓ. In certain embodiments, the free MMAE is less than about 3 ng/mL at Cₘₐₓ.

In certain embodiments, the administration is a single dose administration of the conjugate at a dose between about 1.7 mg/kg to 6.0 mg/kg, inclusive. In certain embodiments, the administration is a single dose administration of the conjugate at a dose between about 1.7 mg/kg to 3.0 mg/kg, inclusive. In certain embodiments, the Cₘₐₓ of the percent free MMAE is determined after administration of a single dose in cycle 1. In a separate embodiment, the Cₘₐₓ of the percent free MMAE is determined in cycle 3 after three separate administrations, three weeks apart of the composition comprising the conjugates of a CLDN6-specific antigen-binding protein covalently bound to heterologous moieties. In certain embodiments, the administration is a single dose of the conjugate between at about 1.7 mg/kg to 3.0 mg/kg, inclusive. In certain embodiments, the conjugate is an anti-CLDN6-specific antibody conjugated to a chemotherapeutic or cytotoxic agent. In certain embodiments, the conjugate is TORL-1-23.

In certain embodiments, the disclosure relates to any of the methods disclosed herein, wherein at a dose normalized Cₘₐₓ value for the unbound (free) circulating MMAE is less than about 35 pg/mL free MMAE per mg of the conjugate after administration of the conjugate, thereby reducing toxicity in the subject and inhibiting the solid tumor. In certain embodiments, the dose normalized Cₘₐₓ value for free MMAE is less than about 30 pg/mL per mg of the conjugate. In certain embodiments the dose normalized Cₘₐₓ value for free MMAE is less than about 25 pg/mL per mg of the conjugate. In certain embodiments, the dose normalized Cₘₐₓ value for free MMAE is less than about 20 pg/mL per mg of the conjugate. In certain embodiments, the dose normalized Cₘₐₓ value for free MMAE is less than about 15 pg/mL per mg of the conjugate. In certain embodiments, the dose normalized Cₘₐₓ value for free MMAE is less than about 10 pg/mL per mg of the conjugate. In certain embodiments, the dose normalized Cₘₐₓ is determined from the Cₘₐₓ measured after a single administration of the conjugate. In certain embodiments, the dose normalized Cₘₐₓ is determined from the Cₘₐₓ measure after three administrations of the same dose with each dose separated by a three-week interval. In certain embodiments, the conjugate is an anti-CLDN6-specific antibody conjugated to a chemotherapeutic or cytotoxic agent. In certain embodiments, the conjugate is TORL-1-23.

In certain embodiments, the disclosure relates to any of the methods disclosed herein, wherein at a Cₘₐₓ after first administration of 3.0 mg/kg the conjugate, the unbound circulating MMAE in serum is less than about 2.6 ng/mL, thereby reducing toxicity in the subject and inhibiting the solid tumor.

In certain embodiments, the disclosure relates to any of the methods disclosed herein, wherein at a Cₘₐₓ after first administration of 2.4 mg/kg of the conjugate, the unbound circulating MMAE in serum is about 4.1±2.1 ng/mL, thereby reducing toxicity in the subject and inhibiting the solid tumor.

In certain embodiments, the disclosure relates to any of the methods disclosed herein, wherein at a Cₘₐₓ after first administration of 2.0 mg/kg of the conjugate, the unbound circulating MMAE in serum is about 3.7±1.6 ng/mL, thereby reducing toxicity in the subject and inhibiting the solid tumor.

In certain embodiments, the disclosure relates to any of the methods disclosed herein, wherein at a Cₘₐₓ after first administration of 1.7 mg/kg of the conjugate, the unbound circulating MMAE in serum is about 2.7±1.9 ng/mL, thereby reducing toxicity in the subject and inhibiting the solid tumor. In certain embodiments, the conjugate is an anti-CLDN6-specific antibody conjugated to a chemotherapeutic or cytotoxic agent. In certain embodiments, the conjugate is TORL-1-23.

In certain embodiments, the disclosure relates to any of the methods disclosed herein, wherein at a cycle 3 Cₘₐₓ after three administrations of 1.7 mg/kg of the conjugate, the unbound circulating MMAE in serum is about 1.4±0.8 ng/mL, wherein the conjugate is administered once every three weeks and cycle 3 starts at the 3^{rd} administration of the conjugate, thereby reducing toxicity in the subject and inhibiting the solid tumor. In certain embodiments, the conjugate is an anti-CLDN6-specific antibody conjugated to a chemotherapeutic or cytotoxic agent. In certain embodiments, the conjugate is TORL-1-23.

In certain embodiments, the disclosure relates to any of the methods disclosed herein, wherein the solid tumor is an ovarian tumor.

In certain embodiments, the disclosure relates to any of the methods disclosed herein, wherein the solid tumor is a bladder tumor.

In certain embodiments, the disclosure relates to any of the methods disclosed herein, wherein the solid tumor is a testicular tumor.

In certain embodiments, the disclosure relates to any of the methods disclosed herein, wherein the solid tumor is an endometrial tumor.

In certain embodiments, the disclosure relates to any of the methods disclosed herein, wherein the solid tumor is non-small cell lung cancer.

In certain embodiments, the disclosure relates to any of the methods disclosed herein, wherein the solid tumor is primary peritoneal cancer.

In certain embodiments, the disclosure relates to any of the methods disclosed herein, wherein the solid tumor is fallopian tube cancer.

In certain embodiments, the disclosure relates to any of the methods disclosed herein, wherein, after administration, the human subject does not experience peripheral neuropathy of grade 3 or higher severity, does not experience alopecia of grade 3 or higher severity, does not experience fatigue of grade 3 or higher severity, does not experience nausea, vomiting or anorexia of grade 3 or higher severity, or does not experience constipation of grade 3 or higher severity. In certain embodiments, the disclosure relates to any of the methods disclosed herein, wherein, after administration, the human subject does not experience any adverse event of grade 3 or higher severity.

In certain embodiments, the disclosure relates to any of the methods disclosed herein, wherein the effective amount is about 1.7 mg/kg to 5 mg/kg, 1.7 mg/kg to 4 mg/kg, or 1.7 mg/kg to 3 mg/kg. In certain embodiments, the disclosure relates to any of the methods disclosed herein, wherein the effective amount is about 2.0 mg/kg to 5 mg/kg, 2.0 mg/kg to 4 mg/kg or 2.0 mg/kg to 3 mg/kg. In certain embodiments, the disclosure relates to any of the methods disclosed herein, wherein the effective amount is about 2.4 mg/kg to 5 mg/kg, 2.4 mg/kg to 4 mg/kg or 2.4 mg/kg to 3 mg/kg. In certain embodiments, the disclosure relates to any of the methods disclosed herein, wherein the effective amount is about 1.7 mg/kg. In certain embodiments, the disclosure relates to any of the methods disclosed herein, wherein the effective amount is about 2.0 mg/kg. In certain embodiments, the disclosure relates to any of the methods disclosed herein, wherein the effective amount is about 2.4 mg/kg. In certain embodiments, the disclosure relates to any of the methods disclosed herein, wherein the effective amount is about 3.0 mg/kg. In certain embodiments, the disclosure relates to any of the methods disclosed herein, wherein the effective amount is about 4 mg/kg. In certain embodiments, the disclosure relates to any of the methods disclosed herein, wherein the effective amount is about 5 mg/kg.

In certain embodiments, the disclosure relates to any of the methods disclosed herein, wherein the effective amount of the composition is administered once every 1-4 weeks, for example once every 2-4 weeks, preferably once every 3 weeks.

In certain embodiments, the disclosure relates to any of the methods disclosed herein, wherein the composition is administered intravenously.

In certain embodiments, the disclosure relates to any of the methods disclosed herein, wherein the effective amount of the composition is administered over a time period from about 20 minutes to about 40 minutes, preferably over a time period of about 30 minutes.

In certain embodiments, the disclosure relates to any of the methods disclosed herein, wherein the human subject achieves a partial response.

In certain embodiments, the disclosure relates to any of the methods disclosed herein, wherein the human subject achieves a complete response.

In certain embodiments, the disclosure relates to any of the methods disclosed herein, wherein the composition further comprises a glutamate-sodium hydroxide buffer, sucrose, and polysorbate 80 (PS80).

In certain embodiments, the disclosure relates to any of the methods disclosed herein, wherein the composition further comprises dextrose and water.

In certain embodiments, the disclosure relates to any of the methods disclosed herein, wherein the composition further comprises sodium chloride and water.

In certain embodiments, the disclosure relates to any of the methods disclosed herein, wherein the average number of heterologous moieties per CLDN6-specific antigen-binding protein in the composition is about 3.5 to about 4, for example, about 3.6 to about 3.9, preferably about 3.7 to about 3.8. In certain embodiments, the disclosure relates to any of the methods disclosed herein, wherein the average number of heterologous moieties per CLDN6-specific antigen-binding protein in the composition is about 3.8 to about 3.9. In certain embodiments, the disclosure relates to any of the methods disclosed herein, wherein the average number of heterologous moieties per CLDN6-specific antigen-binding protein in the composition is at least 3.8.

In certain embodiments, the disclosure relates to any of the methods disclosed herein, wherein, in the first plurality of conjugates, the heterologous moieties are covalently conjugated at unpaired cysteine residues of the CLDN6-specific antigen-binding protein.

In certain embodiments, the disclosure relates to any of the methods disclosed herein, wherein, in the first plurality of conjugates, the heterologous moieties are covalently conjugated at cysteine residues resulting from cleavage of the heavy chain-light chain interchain disulfide bonds of the CLDN6-specific antigen-binding protein.

In certain embodiments, the disclosure relates to any of the methods disclosed herein, wherein about 95%, about 96%, about 97%, or about 98% of the first plurality of conjugates are structurally homogenous.

In certain embodiments, the disclosure relates to any of the methods disclosed herein, wherein a second plurality of the conjugates are bound to eight heterologous moieties comprising structural formula (I).

In certain embodiments, the disclosure relates to any of the methods disclosed herein, wherein a third plurality of the conjugates are bound to two heterologous moieties comprising structural formula (I).

In certain embodiments, the disclosure relates to any of the methods disclosed herein, wherein a fourth plurality of the conjugates are bound to six heterologous moieties comprising structural formula (I).

In certain embodiments, the disclosure relates to any of the methods disclosed herein, wherein the heterologous moiety comprising structural formula (I) has structural formula (II):

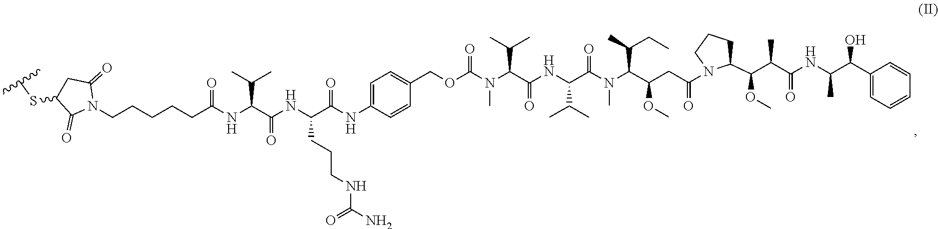 [SMILES: *SC1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C1=O]

wherein

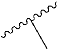

is a covalent thiol bond to the CLDN6-specific antigen-binding protein.

In certain embodiments, the disclosure relates to any of the methods disclosed herein, wherein the subject has not received prior treatment for the solid tumor.

In certain embodiments, the disclosure relates to any of the methods disclosed herein, wherein the subject has received at least one prior treatment for the solid tumor. In certain embodiments, the disclosure relates to any of the methods disclosed herein, wherein the subject has received at least two prior treatments for the solid tumor. In certain embodiments, the disclosure relates to any of the methods disclosed herein, wherein the subject has received at least three prior treatments for the solid tumor. In certain embodiments, the disclosure relates to any of the methods disclosed herein, wherein the subject has received at least four prior treatments for the solid tumor.

In certain embodiments, the disclosure relates to any of the methods disclosed herein, wherein the CLDN6-specific antigen-binding protein is selected from a group consisting of:
(a) a heavy chain CDR1 amino acid sequence set forth in Table A or Table A1, or a variant sequence thereof which differs by only one or two amino acids;
(b) a heavy chain CDR2 amino acid sequence set forth in Table A or Table A1, or a variant sequence thereof which differs by only one or two amino acids;
(c) a heavy chain CDR3 amino acid sequence set forth in Table A or Table A1, or a variant sequence thereof which differs by only one or two amino acids;
(d) a light chain CDR1 amino acid sequence set forth in Table A or Table A1, or a variant sequence thereof which differs by only one or two amino acids;
(e) a light chain CDR2 amino acid sequence set forth in Table A or Table A1, or a variant sequence thereof which differs by only one or two amino acids;
(f) a light chain CDR3 amino acid sequence set forth in Table A or Table A1, or a variant sequence thereof which differs by only one or two amino acids; or a combination of any two or more of (a)-(f).

In certain embodiments, the disclosure relates to any of the methods disclosed herein, wherein the CLDN6-specific antigen-binding protein comprises a heavy chain CDR amino sequence selected from the group consisting of: SEQ ID NOs: 23, 24, 25, 455, 456, 457, and a variant sequence thereof which differs by only one or two amino acids.

In certain embodiments, the disclosure relates to any of the methods disclosed herein, wherein the CLDN6-specific antigen-binding protein comprises a light chain CDR amino sequence selected from the group consisting of: SEQ ID NOs: 20, 21, 22, 476, 477, 454, and a variant sequence thereof which differs by only one or two amino acids.

In certain embodiments, the disclosure relates to any of the methods disclosed herein, wherein the CLDN6-specific antigen-binding protein comprises an antibody that binds CLDN6 or antigen-binding fragment thereof comprising:
(a) a heavy chain CDR1 amino acid sequence set forth in SEQ ID NOs: 23 or 455, or a variant sequence thereof which differs by only one or two amino acids;
(b) a heavy chain CDR2 amino acid sequence set forth in SEQ ID NOs: 24 or 456, or a variant sequence thereof which differs by only one or two amino acids;
(c) a heavy chain CDR3 amino acid sequence set forth in SEQ ID NOs: 25 or 457, or a variant sequence thereof which differs by only one or two amino acids;
(d) a light chain CDR1 amino acid sequence set forth in SEQ ID NOs:20 or 476, or a variant sequence thereof which differs by only one or two amino acids;
(e) a light chain CDR2 amino acid sequence set forth in SEQ ID NOs:21 or 477, or a variant sequence thereof which differs by only one or two amino acids;
(f) a light chain CDR3 amino acid sequence set forth in SEQ ID NOs:22 or 454, or a variant sequence thereof which differs by only one or two amino acids; or
a combination of any two or more of (a)-(f).

In certain embodiments, the disclosure relates to any of the methods disclosed herein, wherein the CLDN6-specific antigen-binding protein comprises an antibody that binds CLDN6 or antigen-binding fragment thereof comprising:
the heavy chain (HC) CDR1-3 amino acid sequences of SEQ ID NOs: 23, 24, and 25, and the light chain (LC) CDR1-3 amino acid sequences of SEQ ID NOs: 20, 21, and 22; or
the heavy chain (HC) CDR1-3 amino acid sequences of SEQ ID NOs: 455, 456, and 457, and the light chain (LC) CDR1-3 amino acid sequences of SEQ ID NOs: 476, 477, and 454.

Additionally, the disclosure includes conjugates of a CLDN6-specific antigen-binding protein covalently bound to heterologous moieties comprising structural formula (I):

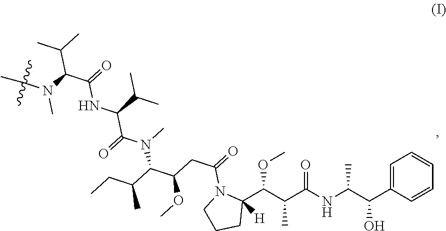 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein
a first plurality of the conjugates are bound to four heterologous moieties comprising structural formula (I);
at least about 95% of the first plurality of conjugates are structurally homogenous; and
the effective amount is in a range of about
   1.7 mg/kg to 6 mg/kg;
   2.0 mg/kg to 6 mg/kg; or
   2.4 mg/kg to 6 mg/kg for use in treating cancer.

In further embodiments, the disclosure relates to any of the methods disclosed herein, wherein the CLDN6-specific antigen-binding protein of the conjugates comprises an antibody that binds CLDN6 or antigen-binding fragment thereof comprising:
(i) HC CDR1 having a sequence GFTFSNYW (SEQ ID NO: 23);
(ii) HC CDR2 having a sequence IRLKSDNYAT (SEQ ID NO: 24),
(iii) HC CDR3 having a sequence XDGPPSGX (SEQ ID NO: 457), wherein X at position 1 is N and X at position 8 is S, T, A, C, or Y,
(iv) LC CDR1 having a sequence ENIYSY (SEQ ID NO: 20),
(v) LC CDR2 having a sequence NAK (SEQ ID NO: 21), and
(vi) LC CDR3 having a sequence QHHYTVPWT (SEQ ID NO: 22).

TORL-1-23 (also referred to as CLDN6-23-ADC) is an antibody drug conjugate comprising an antibody that binds CLDN6 comprising:
(i) HC CDR1 having a sequence GFTFSNYW (SEQ ID NO: 23);
(ii) HC CDR2 having a sequence IRLKSDNYAT (SEQ ID NO: 24),
(iii) HC CDR3 having a sequence XDGPPSGX (SEQ ID NO: 457), wherein X at position 1 is N and X at position 8 is S,
(iv) LC CDR1 having a sequence ENIYSY (SEQ ID NO: 20),
(v) LC CDR2 having a sequence NAK (SEQ ID NO: 21), and
(vi) LC CDR3 having a sequence QHHYTVPWT (SEQ ID NO: 22),
wherein the antibody is conjugated to about four molecules of MMAE, each via a linker comprising Val-Cit-PAB.

### Additional Exemplary Embodiments

1. A method for inhibiting a solid tumor expressing claudin-6 (CLDN6) in a human subject, comprising administering to the human subject an effective amount of a composition comprising conjugates of a CLDN6-specific antigen-binding protein covalently bound to heterologous moieties comprising structural formula (I):

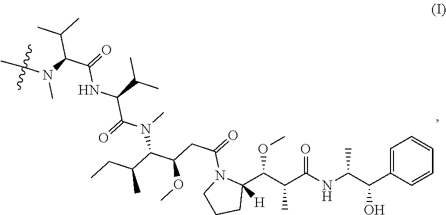 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein
a first plurality of the conjugates are bound to four heterologous moieties comprising structural formula (I);
at least about 95% of the first plurality of conjugates are structurally homogenous; and
the effective amount is in a range of about
   1.7 mg/kg to 6 mg/kg;
   2.0 mg/kg to 6 mg/kg; or
   2.4 mg/kg to 6 mg/kg.

2. The method of 1, wherein at a Cₘₐₓ after administration, the percent of unbound circulating MMAE in serum is less than about 0.01% (w/v), thereby reducing toxicity in the subject and inhibiting the solid tumor.

3. The method of 2, wherein the percent of unbound circulating MMAE is less than about 0.009% (w/v) at Cₘₐₓ.

4. The method of 3, wherein the percent of unbound circulating MMAE is less than about 0.008% (w/v) at Cₘₐₓ.

5. The method of 4, wherein the percent of unbound circulating MMAE is less than about 0.007% (w/v) at Cₘₐₓ.

6. The method of 5, wherein the percent of unbound circulating MMAE is less than about 0.006% (w/v) at Cₘₐₓ.

7. The method of 6, wherein the percent of unbound circulating MMAE is less than about 0.005% (w/v) at Cₘₐₓ.

8. The method of 7, wherein the percent of unbound circulating MMAE is less than about 0.004% (w/v) at Cₘₐₓ.

9. The method of 8, wherein the percent of unbound circulating MMAE is less than about 0.003% (w/v) at Cₘₐₓ.

10. The method of 1, wherein at a Cₘₐₓ after administration, the unbound circulating MMAE in serum is less than about 9 ng/mL, thereby reducing toxicity in the subject and inhibiting the solid tumor.

11. The method of 10, wherein the unbound circulating MMAE is less than about 8 ng/mL at Cₘₐₓ.

12. The method of 11, wherein the unbound circulating MMAE is less than about 7 ng/mL at Cₘₐₓ.

13. The method of 12, wherein the unbound circulating MMAE is less than about 6 ng/mL at Cₘₐₓ.

14. The method of 13, wherein the unbound circulating MMAE is less than about 5 ng/mL at Cₘₐₓ.

15. The method of 14, wherein the unbound circulating MMAE is less than about 4 ng/mL at Cₘₐₓ.

16. The method of 15, wherein the unbound circulating MMAE is less than about 3 ng/mL at Cₘₐₓ.

17. The method of 1, wherein a dose normalized Cₘₐₓ value for the unbound circulating MMAE is less than about 35 pg/mL per mg of the conjugate after administration of the conjugate, thereby reducing toxicity in the subject and inhibiting the solid tumor.

18. The method of 17, wherein the dose normalized Cₘₐₓ value for unbound circulating MMAE is less than about 30 pg/mL per mg of the conjugate.

19. The method of 18, wherein the dose normalized Cₘₐₓ value for unbound circulating MMAE is less than about 25 pg/mL per mg of the conjugate.

20. The method of 19, wherein the dose normalized Cₘₐₓ value for unbound circulating MMAE is less than about 20 pg/mL per mg of the conjugate.

21. The method of 20, wherein the dose normalized Cₘₐₓ value for unbound circulating MMAE is less than about 15 pg/mL per mg of the conjugate.

22. The method of 21, wherein the dose normalized Cₘₐₓ value for unbound circulating MMAE is less than about 10 pg/mL per mg of the conjugate.

23. The method of 1, wherein at a Cₘₐₓ after first administration of 3.0 mg/kg of the conjugate, the unbound circulating MMAE in serum is about 2.6 ng/mL, thereby reducing toxicity in the subject and inhibiting the solid tumor.

24. The method of 1, wherein at a Cₘₐₓ after first administration of 2.4 mg/kg of the conjugate, the unbound circulating MMAE in serum is about 4.1±2.1 ng/mL, thereby reducing toxicity in the subject and inhibiting the solid tumor.

25. The method of 1, wherein at a Cₘₐₓ after first administration of 2.0 mg/kg of the conjugate, the unbound circulating MMAE in serum is about 3.7±1.6 ng/mL, thereby reducing toxicity in the subject and inhibiting the solid tumor.

26. The method of 1, wherein at a Cₘₐₓ after first administration of 1.7 mg/kg of the conjugate, the unbound circulating MMAE in serum is about 2.7±1.9 ng/mL, thereby reducing toxicity in the subject and inhibiting the solid tumor.

27. The method of 1, wherein cycle 3 Cₘₐₓ after three administrations of 1.7 mg/kg of the conjugate, the unbound circulating MMAE in serum is about 1.4±0.8 ng/mL, wherein the conjugate is administered once every three weeks and cycle 3 starts at the 3^{rd} administration of the conjugate, thereby reducing toxicity in the subject and inhibiting the solid tumor.

28. The method of any one of 1-27, wherein the solid tumor is an ovarian tumor.

29. The method of any one of 1-27, wherein the solid tumor is a bladder tumor.

30. The method of any one of 1-27, wherein the solid tumor is a testicular tumor.

31. The method of any one of 1-27, wherein the solid tumor is an endometrial tumor.

32. The method of any one of 1-27, wherein the solid tumor is non-small cell lung cancer.

33. The method of any one of 1-27, wherein the solid tumor is primary peritoneal cancer.

34. The method of any one of 1-27, wherein the solid tumor is fallopian tube cancer.

35. The method of any one of 1-34, wherein, after administration, the human subject does not experience peripheral neuropathy of grade 3 or higher severity, does not experience alopecia of grade 3 or higher severity, does not experience fatigue of grade 3 or higher severity, does not experience nausea, vomiting or anorexia of grade 3 or higher severity, or does not experience constipation of grade 3 or higher severity.

36. The method of any one of 1-35, wherein, after administration, the human subject does not experience any adverse event of grade 3 or higher severity.

37. The method of any one of 1-36, wherein the effective amount is about 1.7 mg/kg to 5 mg/kg, 1.7 mg/kg to 4 mg/kg, or 1.7 mg/kg to 3 mg/kg.

38. The method of any one of 1-36, wherein the effective amount is about 2.0 mg/kg to 5 mg/kg, 2.0 mg/kg to 4 mg/kg or 2.0 mg/kg to 3 mg/kg.

39. The method of any one of 1-36, wherein the effective amount is about 2.4 mg/kg to 5 mg/kg, 2.4 mg/kg to 4 mg/kg or 2.4 mg/kg to 3 mg/kg.

40. The method of any one of 1-36, wherein the effective amount is about 1.7 mg/kg.

41. The method of any one of 1-36, wherein the effective amount is about 2.0 mg/kg.

42. The method of any one of 1-36, wherein the effective amount is about 2.4 mg/kg.

43. The method of any one of 1-36, wherein the effective amount is about 3.0 mg/kg.

44. The method of any one of 1-36, wherein the effective amount is about 3.6 mg/kg.

45. The method of any one of 1-36, wherein the effective amount is about 4 mg/kg.

46. The method of any one of 1-45, wherein the effective amount of the composition is administered once every 1-4 weeks, for example once every 2-4 weeks, preferably once every 3 weeks.

47. The method of any one of 1-46, wherein the composition is administered intravenously.

48. The method of any one of 1-47, wherein the effective amount of the composition is administered over a time period from about 20 minutes to about 40 minutes, preferably over a time period of about 30 minutes.

49. The method of any one of 1-48, wherein the human subject achieves a partial response.

50. The method of any one of 1-48, wherein the human subject achieves a complete response.

51. The method of any one of 1-50, wherein the composition further comprises a glutamate-sodium hydroxide buffer, sucrose, and polysorbate 80 (PS80).

52. The method of any one of 1-51, wherein the composition further comprises dextrose and water.

53. The method of any one of 1-51, wherein the composition further comprises sodium chloride and water.

54. The method of any one of 1-53, wherein the average number of heterologous moieties per CLDN6-specific antigen-binding protein in the composition is about 3.5 to about 4, for example, about 3.6 to about 3.9, preferably about 3.7 to about 3.8.

55. The method of any one of 1-54, wherein, in the first plurality of conjugates, the heterologous moieties are covalently conjugated at unpaired cysteine residues of the CLDN6-specific antigen-binding protein.

56. The method of any one of 1-54, wherein, in the first plurality of conjugates, the heterologous moieties are covalently conjugated at cysteine residues resulting from cleavage of the heavy chain-light chain interchain disulfide bonds of the CLDN6-specific antigen-binding protein.

57. The method of any one of 1-56, wherein about 95%, about 96%, about 97%, or about 98% of the first plurality of conjugates are structurally homogenous.

58. The method of any one of 1-57, wherein the heterologous moiety comprising structural formula (I) has structural formula (II):

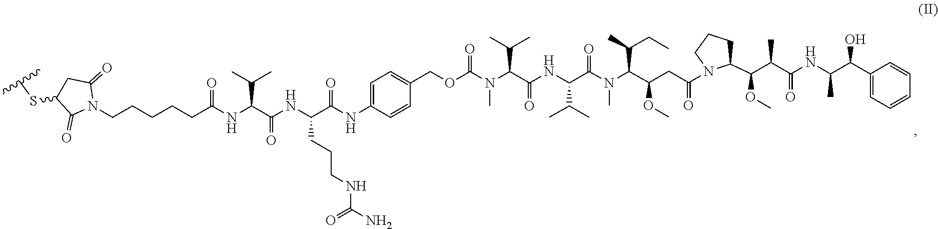 [SMILES: *SC1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C1=O]

wherein

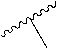

is a covalent thiol bond to the CLDN6-specific antigen-binding protein.

59. The method of any one of 1-58, wherein the subject has not received prior treatment for the solid tumor.

60. The method of any one of 1-58, wherein the subject has received at least one prior treatment for the solid tumor.

61. The method of any one of 1-58, wherein the subject has received at least two prior treatments for the solid tumor.

62. The method of any one of 1-58, wherein the subject has received at least three prior treatments for the solid tumor.

63. The method of any one of 1-58, wherein the subject has received at least four prior treatments for the solid tumor.

64. The method of anyone of 1-63, wherein the effective amount is in the range of 3.0 mg/kg to 3.6 mg/kg.

65. The method of any one of 1-64, wherein prior to administering to the human subject an effective amount of the composition, the human subject is administered an effective amount of a G-CSF.

66. The method of any one of 1-65, wherein the CLDN6-specific antigen-binding protein is selected from a group consisting of:
(a) a heavy chain CDR1 amino acid sequence set forth in Table A or Table A1, or a variant sequence thereof which differs by only one or two amino acids;
(b) a heavy chain CDR2 amino acid sequence set forth in Table A or Table A1, or a variant sequence thereof which differs by only one or two amino acids;
(c) a heavy chain CDR3 amino acid sequence set forth in Table A or Table A1, or a variant sequence thereof which differs by only one or two amino acids;
(d) a light chain CDR1 amino acid sequence set forth in Table A or Table A1, or a variant sequence thereof which differs by only one or two amino acids;
(e) a light chain CDR2 amino acid sequence set forth in Table A or Table A1, or a variant sequence thereof which differs by only one or two amino acids;
(f) a light chain CDR3 amino acid sequence set forth in Table A or Table A1, or a variant sequence thereof which differs by only one or two amino acids; or
a combination of any two or more of (a)-(f).

67. The method of 66, wherein the CLDN6-specific antigen-binding protein comprises an antibody that binds CLDN6 or antigen-binding fragment thereof comprising:
(a) the heavy chain (HC) CDR1-3 amino acid sequences of SEQ ID NOs: 23, 24, and 25, and the light chain (LC) CDR1-3 amino acid sequences of SEQ ID NOs: 20, 21, and 22; or
(b) the heavy chain (HC) CDR1-3 amino acid sequences of SEQ ID NOs: 455, 456, and 457, and the light chain (LC) CDR1-3 amino acid sequences of SEQ ID NOs: 476, 477, and 454.

68. The method of any one of 1-67, wherein the CLDN6-specific antigen-binding protein comprises an antibody that binds CLDN6 or antigen-binding fragment thereof comprising:
(i) HC CDR1 having a sequence GFTFSNYW (SEQ ID NO: 23);
(ii) HC CDR2 having a sequence IRLKSDNYAT (SEQ ID NO: 24),
(iii) HC CDR3 having a sequence XDGPPSGX (SEQ ID NO: 457), wherein X at position 1 is N and X at position 8 is S, T, A, C, or Y,
(iv) LC CDR1 having a sequence ENIYSY (SEQ ID NO: 20),
(v) LC CDR2 having a sequence NAK (SEQ ID NO: 21), and
(vi) LC CDR3 having a sequence QHHYTVPWT (SEQ ID NO: 22).

69. The method of 68, wherein the CLDN6-specific antigen-binding protein comprises: HC CDR3 having a sequence XDGPPSGX (SEQ ID NO: 457), wherein X at position 1 is N and X at position 8 is S.

70. The method of any one of 1-67, wherein the CLDN6-specific antigen-binding protein comprises an antibody that binds CLDN6 or antigen-binding fragment thereof comprising:
(i) CDRs1-3 from a heavy chain variable region comprising the amino acid sequence set forth in SEQ ID NO: 387; and
(ii) CDRs1-3 from a light chain variable region comprising the amino acid sequence set forth in SEQ ID NO: 389.

71. A conjugate of a CLDN6-specific antigen-binding protein covalently bound to heterologous moieties comprising structural formula (I):

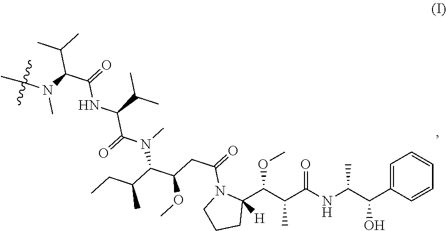 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein
a first plurality of the conjugates are bound to four heterologous moieties comprising structural formula (I);
at least about 95% of the first plurality of conjugates are structurally homogenous; and
the effective amount is in a range of about
   1.7 mg/kg to 6 mg/kg;
   2.0 mg/kg to 6 mg/kg; or
   2.4 mg/kg to 6 mg/kg for use in treating cancer.

### SEQUENCES

The following sequences were part of the Sequence Listing filed as Appendix 3 in the U.S. Application No. 63/468,817 to which this application claims priority. These sequences are less than 4 amino acids or 10 base pairs in length, and do not meet the length requirements of ST. 26, paragraph 7.

SEQ ID NO: 9
Ser Thr Ser
SEQ ID NO: 15
Asn Ala Lys
SEQ ID NO: 21
Asn Ala Lys
SEQ ID NO: 27
Lys Val Ser
SEQ ID NO: 33
Lys Val Ser
SEQ ID NO: 39
Trp Ala Ser
SEQ ID NO: 45
Ser Thr Ser
SEQ ID NO: 51
Lys Val Ser
SEQ ID NO: 57
Ala Ala Ala
SEQ ID NO: 63
Lys Val Ser
SEQ ID NO: 69
Leu Ala Ser
SEQ ID NO: 75
Ser Thr Ser
SEQ ID NO: 81
Arg Thr Ser
SEQ ID NO: 87
Ala Ala Ser
SEQ ID NO: 93
Trp Ala Ser
SEQ ID NO: 99
Ala Ala Ser
SEQ ID NO: 105
Ser Thr Ser
SEQ ID NO: 111
Trp Ala Ser
SEQ ID NO: 117
Trp Ala Ser
SEQ ID NO: 123
Lys Val Ser
SEQ ID NO: 129
Ser Thr Ser
SEQ ID NO: 209
agcacatcc
SEQ ID NO: 215
aatgcaaaa
SEQ ID NO: 221
aatgcaaaa
SEQ ID NO: 227
aaagtttcc
SEQ ID NO: 233
aaagtttcc
SEQ ID NO: 239
tgggcatcc
SEQ ID NO: 245
agcacatcc
SEQ ID NO: 251
aaagtttcc
SEQ ID NO: 257
gctgcagcc
SEQ ID NO: 263
aaagtttcc
SEQ ID NO: 269
cttgcatcc
SEQ ID NO: 275
tccacatcc
SEQ ID NO: 281
aggacatcc
SEQ ID NO: 287
gctgcatcc
SEQ ID NO: 293
tgggcatcc
SEQ ID NO: 299
gctgcatcc
SEQ ID NO: 305
agcacatcc
SEQ ID NO: 311
tgggcatcc
SEQ ID NO: 317
tgggcatcc
SEQ ID NO: 323
aaagtttcc
SEQ ID NO: 329
tccacatcc
SEQ ID NO: 450
Xaa Thr Xaa, wherein Xaa at position 1 is S, T,
Q, or A, and Xaa at position 3 is S, T, D, or Q.
SEQ ID NO: 459
Xaa Val Xaa, wherein Xaa at position 1 is K, Q,
or R, and Xaa at position 3 is S, T, or V.
SEQ ID NO: 471
Xaa Ala Ser, wherein Xaa at position 1 is H, Y,
F, or W.
SEQ ID NO: 477
Xaa Ala Lys, wherein Xaa at position 1 is Q, S,
A, D, or N.

### EXAMPLES

The following examples are given merely to illustrate the present disclosure and not in any way to limit its scope.

### Example 1

This example describes administration of a CLDN6 ADC for treatment of cancer in humans.

### Participant Inclusion Criteria

1. Female or male ≥18 years of age willing and able to provide informed consent

### 2. Disease Type:

For Part 1:
Histologically or cytologically confirmed diagnosis of advanced (unresectable) or metastatic solid tumor malignancy that was not responsive to accepted standard therapies or for which there was no standard therapy
For Part 2 Cohort 2A:
Histologically or cytologically confirmed diagnosis of advanced (unresectable) or metastatic ovarian, primary peritoneal, or fallopian tube cancer
Patient's tumor must be positive for claudin 6 expression as defined by the claudin 6 reference laboratory assay, an IHC assay.
Patients must have platinum-resistant disease (defined as progression within 6 months from completion of a minimum of four cycles of platinum-containing therapy). Note: This should be calculated from the date of the last administered dose of platinum therapy to the date of the radiographic imaging showing progression. Patients who are platinum-refractory during front-line treatment are excluded.
Patients must have received at least 1 but no more than 4 prior systemic lines of anticancer therapy, and for whom single-agent therapy is appropriate as the next line of treatment:
   a. Adjuvant±neoadjuvant considered one line of therapy
   b. Maintenance therapy (e.g., bevacizumab, PARP inhibitors) will be considered as part of the preceding line of therapy (ie, not counted independently)
   c. Therapy changed due to toxicity in the absence of progression will be considered as part of the same line (i.e., not counted independently)
   d. Hormonal therapy will not be counted as a separate line of therapy.
For Part 2 Cohort 2B:
Histologically or cytologically confirmed diagnosis of advanced (unresectable) or metastatic solid tumor malignancy that is not responsive to accepted standard therapies or for which there is no standard therapy
Patient's tumor must be positive for claudin 6 expression as defined by the claudin 6 reference laboratory assay.
For Part 2 Cohort 2C:
Histologically confirmed diagnosis of advanced (unresectable) or metastatic NSCLC that has progressed on or following treatment with platinum-based regimens and PD1 or PDL1 inhibitor containing regimens and EGFR TKI containing regimen for EGFRmu NSCLC and ALK inhibitor regimen for ALK translocated NSCLC and KRAS G12C inhibitor for KRASG12Cmu NSCLC
Patient's tumor must be positive for claudin 6 expression as defined by the claudin 6 reference laboratory assay
   3. Measurable disease, per RECIST v1.1
   4. Eastern Cooperative Oncology Group (ECOG) performance status 0-1
   5. Able to tolerate intravenous blood sampling for PK, had no known intolerance or hypersensitivity to IMP or excipients, and able to comply with study requirements
   6. Adequate organ function, based on the following laboratory values, as shown in Table 2.

TABLE 2
Adequate organ function criteria.

| | |
|---|---|
| System | Laboratory Value |

Hematological

| | |
|---|---|
| ANC | ≥1,500/mcL |
| Platelets | ≥100,000/mcL without transfusion within 4 |
| | weeks of first dose |
| Hemoglobin | ≥8 g/dL |

Renal

| | |
|---|---|
| Measured or Cockcroft | ≥30 mL/min |
| and Gault equation | |
| calculated creatinine | |
| clearance | |

Hepatic

| | |
|---|---|
| Serum total bilirubin | ≤1.5 × ULN (participants with known Gilbert |
| | disease who have serum bilirubin level ≤3 × |
| | ULN may be enrolled) |
| AST (SCOT) and | ≤3 × ULN |
| ALT (SGPT) | |
| Albumin | ≥2 g/dL |

Cardiac

| | |
|---|---|
| ECG | 12-Lead ECG with normal tracing or non- |
| | clinically significant changes that do not require |
| | medical intervention and QTcF interval ≤470 |
| | msec and without history of Torsades des Pointes |
| | or other symptomatic QTc abnormality. |

ALT = alanine aminotransferase;
ANC = absolute neutrophil count;
AST = aspartate aminotransferase;
ECG = electrocardiogram;
QTc = corrected QT interval;
QTcF = QT interval corrected by Friderica's formula;
ULN = upper limit of normal

7. Female participants of childbearing potential must have a negative urine or serum pregnancy test within 72 hours before starting study drug treatment. If the urine test is positive or cannot be confirmed as negative, a serum pregnancy test will be required. The serum pregnancy test must be negative for the participant to be eligible, and participants must agree to use a highly effective birth control method from the time of the first study drug treatment through 90 days after the last study drug treatment, or be of nonchildbearing potential. Highly effective birth control method is defined as combined (estrogen and progestogen containing) hormonal contraception (e.g., oral, intravaginal, transdermal), progestin-only hormonal contraception associated with inhibition of ovulation (e.g., oral, injectable, implantable, intrauterine device [IUD], intrauterine hormone-releasing system [IUS]), bilateral tubal occlusion, vasectomized partner, or sexual abstinence. Abstinence refers to ‘true abstinence,’ which means it is in line with the preferred and usual lifestyle of the patient. Periodic abstinence (e.g., calendar, ovulation, symptothermal, post-ovulation methods), declaration of abstinence for the duration of exposure to study treatment, and withdrawal are not acceptable methods of contraception. Nonchildbearing potential is defined as follows:
a. ≥45 years of age and has not had menses for >1 year
b. Participants who have been amenorrhoeic for <2 years without history of a hysterectomy and oophorectomy must have a follicle stimulating hormone (FSH) value in the postmenopausal range upon screening evaluation
c. Post-hysterectomy, post-bilateral oophorectomy, or post-tube ligation. Documented hysterectomy or oophorectomy must be confirmed with medical records of the actual procedure or confirmed by an ultrasound. Tubal ligation must be confirmed with medical records of the actual procedure.
   8. Female and male participants must agree not to donate eggs or sperm, respectively, from the first study-drug treatment through 90 days after the last study drug treatment.
   9. Female participants must agree to not breastfeed from the first dose of study treatment through 90 days after the last dose of study treatment.
   10. Male participants must use a condom when having sex with a pregnant woman and when having sex with a woman of childbearing potential from the time of the first study-drug treatment through 4 months after the last study drug treatment. Withdrawal (coitus interruptus) and/or use of a spermicide without a condom are not acceptable methods of contraception or fetal protection. Effective contraception should be considered for a non-pregnant female partner of childbearing potential. Male participants are advised to have semen samples frozen before initiation of TORL-1-23 administration given the potential risk to male fertility.

### Participant Exclusion Criteria

1. Has not recovered [recovery is defined as NCI CTCAE, version 5.0, grade ≤1] from the acute toxicities of previous therapy, except treatment-related alopecia or laboratory abnormalities otherwise meeting eligibility requirements

### 2. For Cohort 2A

a. Patients with low-grade, borderline ovarian tumors or non-epithelial ovarian cancers
b. Patients with primary platinum-refractory ovarian, primary peritoneal or fallopian tube cancer, defined as disease that did not respond to or has progressed within 3 months of the last dose of first line platinum-containing chemotherapy
   3. Received prior chemotherapeutic, investigational, radiotherapy, or other therapies for the treatment of cancer within 14 days with small molecule and within 28 days with biologic before the first dose of TORL-1-23. There is no waiting period required for stereotactic radiosurgery (SRS).
   4. Progressive or symptomatic brain metastases. Brain metastases that have been radiated, are asymptomatic, and on a stable or decreasing dose of steroids are allowed. Leptomeningeal disease is excluded.
   5. Grade 2 or greater peripheral neuropathy
   6. Participants must not be considered a poor medical risk due to a serious, uncontrolled medical disorder, nonmalignant systemic disease, or active, uncontrolled infection. Examples include, but are not limited to, uncontrolled major seizure disorder, unstable spinal cord compression, superior vena cava syndrome, or any psychiatric disorder that prohibits obtaining informed consent.
   7. History of significant cardiac disease:
Congestive heart failure >New York Heart Association (NYHA) class 2 within last year
Unstable angina (angina symptoms at rest), new-onset angina (begun within the last 3 months)
Myocardial infarction less than 6 months before start of study drug
Anti-arrhythmic therapy (beta blockers are permitted)
Any unstable ischemic disease or untreated arrhythmia
   8. Known history of myelodysplastic syndrome (MDS) or acute myeloid leukemia (AML)
   9. History of another cancer within 3 years before Day 1 of study treatment, with the exception of basal or squamous cell carcinoma of the skin that has been definitively treated. Participants with malignancies with a low risk of recurrence, including appropriately treated ductal carcinoma in situ (DCIS) of the breast and prostate cancer with a Gleason score less than or equal to 6, are not excluded.
   10. Uncontrolled infection; active, clinically serious infections (>CTCAE grade 2)
   11. Participants with seizure disorder requiring medication
   12. Participants must not be pregnant or breastfeeding
   13. Known hypersensitivity or intolerance to any of the study drugs, study drug classes, or excipients in the formulation
   14. History of having an allogeneic bone marrow or organ transplant
   15. Any condition (concurrent disease, infection, or comorbidity) that interferes with ability to participate in the study, causes undue risk, or complicates the interpretation of safety data, in the opinion of the Investigator.
   16. Participants who are taking any drugs that are strong inducers and/or strong inhibitors of CYP3A4 enzymes
   17. Participants who are taking any drugs that are inhibitors of P-glycoprotein (P-gp)

### Administration and Monitoring Protocols

The drug product of TORL-1-23 is a sterile, colorless to slightly yellow, clear to slightly opalescent liquid that is essentially free of visible particles and was supplied in a 10 mL glass vial with a flip-off seal (cap) over a 20 mm rubber stopper. Each vial contained 40 mg of TORL-1-23. Drug product was formulated at a target concentration of 10 mg/mL in 20 mM Glutamate-NaOH buffer, 8% (w/v) Sucrose, 0.02% (w/v) PS80, pH 5.2. Transfer and dilution to an intravenous (IV) bag was required prior to IV infusion.

The appropriate volume of TORL-1-23 for injection was transferred to a non-PVC IV infusion bag containing USP grade 5% Dextrose Injection or 0.9% Sodium Chloride Injection. The final concentration of the prepared TORL-1-23 dosing solution in 5% Dextrose was ≥0.1 mg/mL and ≤4 mg/mL. If diluting into 0.9% Sodium Chloride, the final concentration of the prepared TORL-1-23 was ≥0.2 mg/mL and ≤4 mg/mL

Dosing was based on participant weight. The investigational product will be administered at mg/kg doses based on the participant's actual body weight at baseline. The dose was adjusted if the participant's weight changed by ≥10% from their baseline weight.

The investigational product was administered through a dedicated IV line with a 0.22 micron in-line filter.

TORL-1-23 was administered as a 30 minute IV infusion once every 3 weeks on 21-day cycles (±3 days after Cycle 1). See description of Parts 1 and 2 below for specific treatment group descriptions.

Participants were continuously monitored for AEs throughout the duration of the study. Complete safety assessments (physical examination, vital sign measurements, 12-lead ECGs, and clinical laboratory tests) were performed periodically at baseline, on-treatment, and at follow-up. Widely accepted criteria for documentation and classification of adverse events were utilized [i.e., National Cancer Institute (NCI) Common Terminology Criteria for Adverse Events (CTCAE) Version 5.0].

Participants experiencing Grade 3 possibly related toxicity or intolerable Grade 2 toxicity despite optimal supportive care had their treatment interrupted until resolution to ≤Grade 1 (except for alopecia, fatigue, or peripheral neuropathy). Upon adequate recovery, treatment was resumed at the next lowest dose level.

Tumor assessments by Response Evaluation Criteria in Solid Tumors (RECIST) v1.1 were performed at Screening, and every 6 to 9 weeks after Cycle 1 Day 1 in the first year and at least every 12 weeks (±4 weeks) thereafter or as clinically indicated until disease progression or withdrawal from the study. PK parameters were determined for TORL-1-23 during Cycle 1.

Safety Follow-up Period: Participants were followed for ongoing and new adverse events for 28 days after the last investigational medicinal product (IMP) administration, or until all drug-related toxicities resolved or were deemed stable, whichever was later.

Survival Follow-up Period: All participants will be followed at least every 3 months (±1 month) for up to 2 years for survival and new systemic anticancer treatment. Participants who do not return to the site should be contacted by telephone every 3 months as an alternative.

### Part 1: Dose Escalation

The starting dose level was 0.2 mg/kg IV infusion once every 3 weeks.

In order to reduce the number of participants who were treated at sub-therapeutic dose levels while ensuring a risk mitigated starting dose and dose escalation, dose finding was conducted according to an accelerated titration design by Simon et al. (1997). During the accelerated phase, sequential cohorts of 1 participant each using 100% dose increments were evaluated for 21 days or until the first instance of DLT or first course CTCAE Grade 2 or greater possibly related toxicity. At dose levels above 0.8 mg/kg (2 dose doublings from the starting dose) or with a DLT or first course Grade 2 or greater possibly related AE, the accelerated phase of dose finding reverted to a standard 3+3 dose escalation design using ˜33% or less dose increments between all subsequent dose cohorts.

At each dose level in the standard 3+3 escalation phase, at least 3 participants were or will be treated (unless DLT is observed in the first 2 participants at a dose level). If a DLT is observed in 1 of the initial 3 treated participants at a dose level in the standard 3+3 dose escalation phase, 3 additional participants were or will be enrolled and treated at the same dose level. If no further DLT is observed, the next dose level may be opened using a ˜33% or less dose increment. Subsequent dose levels may not be opened until all participants entered at the current dose level have been treated and observed for at least one complete 21-day cycle and the number of participants with DLTs in their 4-week cycle has been determined. Dose escalation continued and will continue, as tolerated, until 6.0 mg/kg, DLT is observed in at least 2 of the 3 to 6 participants treated at that dose level, or potentially at a lower dose if the MTD has been reached.

The treatment groups for the cohorts are listed in Table 3 based on this information.

TABLE 3
Monotherapy Dose Escalation - Part 1

| Cohort | TORL-1-23 Dose |
|---|---|
| P1-1 | 0.2 mg/kg iv infusion once every 3 weeks |
| P1-2 | 0.4 mg/kg iv infusion once every 3 weeks |
| P1-3 | 0.8 mg/kg iv infusion once every 3 weeks |
| P1-4 | 1.0 mg/kg iv infusion once every 3 weeks |
| P1-5 | 1.3 mg/kg iv infusion once every 3 weeks |
| P1-6 | 1.7 mg/kg iv infusion once every 3 weeks |
| P1-7 | 2.0 mg/kg iv infusion once every 3 weeks |
| P1-8 | 2.4 mg/kg iv infusion once every 3 weeks |
| P1-9 | 3.0 mg/kg iv infusion once every 3 weeks |
| P1-10 | 4.0 mg/kg iv infusion once every 3 weeks |
| P1-11 | 5.0 mg/kg iv infusion once every 3 weeks |
| P1-12 | 6.0 mg/kg iv infusion once every 3 weeks |

^{a}100% dose escalations between cohorts until dose level exceeded 0.8 mg/kg every 3 weeks (2 dose doublings from the starting dose) or a DLT or Grade 2 possibly related toxicities are observed during Cycle 1, at which point dose escalation reverted to ˜33% or less dose increments between cohorts until MTD was reached and RP2D was declared.

DLT was defined as any of the following events as classified according to NCI CTCAE, version 5.0, which are not clearly due to underlying disease or extraneous causes (regardless of investigator attribution) and occur during the first 21 days of Cycle 1:

Any death not clearly due to underlying disease or extraneous causes
≥Grade 4 hematologic toxicity
≥Grade 3 febrile neutropenia
≥Grade 3 thrombocytopenia with bleeding
≥Grade 3 non-hematologic toxicity except:
untreated diarrhea, nausea, vomiting, constipation, pain, and rash which only become DLTs if the AE persists for ≥72 hours despite adequate treatment
fatigue which only becomes DLT if persists ≥1 week
electrolyte abnormality which only become DLT if lasts >72 hours, unless the patient has clinical symptoms, in which case all grade 3+ electrolyte abnormality regardless of duration should count as a DLT
amylase or lipase elevations which only becomes DLT if associated with symptoms or clinical manifestations of pancreatitis
for participants with hepatic metastases, aspartate aminotransferase (AST) or alanine aminotransferase (ALT) elevations which only become DLT if AST or ALT >8× upper limit of normal (ULN) or AST or ALT >5×ULN for ≥14 days
   AEs that are cases of Hy's law (e.g., ALT or AST >3×ULN and total bilirubin [TBILI]>2×ULN without initial findings of cholestasis [elevated serum alkaline phosphatase (ALP)])
   Inability to begin Cycle 2 for >14 days due to TORL-1-23-related toxicity

### Results

To date, 25 participants have been enrolled in this study and treated across 8 dose levels, ranging from 0.2 to 2.4 mg/kg IV every 3 weeks. 95% of patients had received at least 3 prior lines of treatment in the metastatic setting. Table 4 shows the demographics of the 25 participants.

TABLE 4
Demographics of Phase 1 Clinical Trial Participants

| Dose, mg/kg | 0.2 | 0.4 | 0.8 | 1 | 1.3 | 1.7 | 2 | 2.4 | Total |
|---|---|---|---|---|---|---|---|---|---|
| N | 1 | 1 | 1 | 3 | 3 | 5^{a} | 5^{a} | 6^{a} | 25 |
| Age, years | 56 | 44 | 57 | 50 (30- | 55 (48- | 62 (52- | 51 (27- | 66 (59- | 61 (27- |
| (range) | | | | 69) | 70) | 66) | 65) | 74) | 74) |
| Gender, | 0/1 | 1/0 | 0/1 | 1/2 | 0/3 | 0/5 | 1/4 | 0/6 | 3/22 |
| male/female | | | | | | | | | |
| Cancer type, n | | | | | | | | | |
| Ovarian | 1 | 0 | 1 | 2 | 3 | 4 | 4 | 4 | 19 |
| Testicular | 0 | 1 | 0 | 1 | 0 | 0 | 1 | 0 | 3 |
| Endometrial | 0 | 0 | 0 | 0 | 0 | 1 | 0 | 2 | 3 |
| Median number | 6 | 3 | 10 | 5 (1- | 6 (6- | 5 (3- | 6 (3- | 4 (1- | 5 (1- |
| of prior | | | | 9) | 7) | 7) | 9) | 6) | 10) |
| treatments | | | | | | | | | |
| (range) | | | | | | | | | |
| CLDN6 Positive | 1/1 | 1/1 | 1/1 | 3/3 | 3/3 | 3/5 | 5/5 | 5/6 | 22/25 |
| by IHC n/N | | | | | | | | | |

^{a}Cohort expanded to further characterize and guide dose selection

No dose-limiting toxicities (DLTs) have been reported. The most common treatment-related adverse events were grade 1 peripheral neuropathy (n=3), grade 1 alopecia (n=3), grade 1/2 fatigue (n=5), grade 1 anemia (n=2), grade 3 anemia (n=2), and grade 1/2 nausea (n=3), as shown in Table 5.

TABLE 5
Treatment Related Adverse Events Occurring in 10% or Greater

| | | 0.2 mg/kg | 0.4 mg/kg | 0.8 mg/kg | 1 mg/kg | 1.3 mg/kg | 1.7 mg/kg | 2 mg/kg | 2.4 mg/kg | Total |
|---|---|---|---|---|---|---|---|---|---|---|
| Preferred | | N = 1; | N = 1; | N = 1; | N = 3; | N = 3; | N = 5; | N = 5; | N = 6; | N = 25 |
| Term | | n(%) | n(%) | n(%) | n(%) | n(%) | n(%) | n(%) | n(%) | n(%) |
| Patients | | 1 (100) | 1 (100) | 1 (100) | 2 (66) | 1 (33) | 3 (60) | 2 (40) | 3 (50) | 14 (56) |
| with any | | | | | | | | | | |
| TRAE | | | | | | | | | | |
| Alopecia | All | 1 (100) | 0 | 0 | 0 | 0 | 0 | 1 (20) | 1 (17) | 3 (12) |
| | Grades | | | | | | | | | |
| | Gr 1 | 1 (100) | 0 | 0 | 0 | 0 | 0 | 1 (20) | 1 (17) | 3 (12) |
| | Gr 2 | 0 | 0 | 0 | 0 | 0 | 0 | 0 | 0 | 0 |
| | Gr 3 | 0 | 0 | 0 | 0 | 0 | 0 | 0 | 0 | 0 |
| Anaemia | All | 0 | 0 | 0 | 1 (33) | 0 | 1 (20) | 0 | 2 (33) | 4 (16) |
| | Grades | | | | | | | | | |
| | Gr 1 | 0 | 0 | 0 | 0 | 0 | 1 (20) | 0 | 1 (17) | 2 (8) |
| | Gr 2 | 0 | 0 | 0 | 0 | 0 | 0 | 0 | 0 | 0 |
| | Gr 3 | 0 | 0 | 0 | 1 (33) | 0 | 0 | 0 | 1 (17) | 1 (4) |
| Fatigue | All | 0 | 0 | 1 (100) | 1 (33) | 0 | 3 (60) | 0 | 0 | 5 (20) |
| | Grades | | | | | | | | | |
| | Gr 1 | 0 | 0 | 1 (100) | 1 (33) | 0 | 1 (20) | 0 | 0 | 3 (12) |
| | Gr 2 | 0 | 0 | 0 | 0 | 0 | 2 (40) | 0 | 0 | 2 (8) |
| | Gr 3 | 0 | 0 | 0 | 0 | 0 | 0 | 0 | 0 | 0 |
| Nausea | All | 0 | 0 | 0 | 0 | 0 | 0 | 0 | 1 (17) | 1 (4) |
| | Grades | | | | | | | | | |
| | Gr 1 | 0 | 0 | 0 | 0 | 0 | 1 (20) | 0 | 1 (17) | 1 (4) |
| | Gr 2 | 0 | 0 | 0 | 0 | 0 | 1 (20) | 0 | 0 | 1 (4) |
| | Gr 3 | 0 | 0 | 0 | 0 | 0 | 0 | 0 | 0 | 0 |
| Neuropathy | All | 0 | 1 (100) | 1 (100) | 1 (33) | 0 | 0 | 0 | 0 | 3 (12) |
| peripheral | Grades | | | | | | | | | |
| | Gr 1 | 0 | 1 (100) | 1 (100) | 1 (33) | 0 | 0 | 0 | 0 | 3 (12) |
| | Gr 2 | 0 | 0 | 0 | 0 | 0 | 0 | 0 | 0 | 0 |
| | Gr 3 | 0 | 0 | 0 | 0 | 0 | 0 | 0 | 0 | 0 |
| Dose Limiting | | | | | | | | | | |
| Toxicities | | | | | | | | | | |
| (DLT) | | | | | | | | | | |
| DLT | All | 0 | 0 | 0 | 0 | 0 | 0 | 0 | 0 | 0 |

Grade 4 Lymphocyte count decreased (n = 1), Grade 5 Pneumonia (n = 1).
Datacut 3 May 2023 Dose Levels 0.2 to 2.4 mg/kg

No dose reductions or delays for toxicity were required. Preliminary PK data demonstrate sustained exposure of TORL-1-23 over the dosing interval and low levels of free MMAE.

Confirmed partial responses (PR) were observed in 7/25 evaluable participants, including in 6/19 subjects with platinum-resistant/refractory ovarian cancer across all dose levels, with 3/4 patients at the 2.4 mg/kg dose level achieving a PR. Separately, 1/3 patients with metastatic CLDN6 positive testicular cancer achieved a PR; this patient previously was treated with 4 prior lines of treatment. This participant initially received 1 mg/kg and was subsequently escalated to 1.3 mg/kg; the participant remains in response in Cycle 11 and is currently being treated with 1.7 mg/kg.

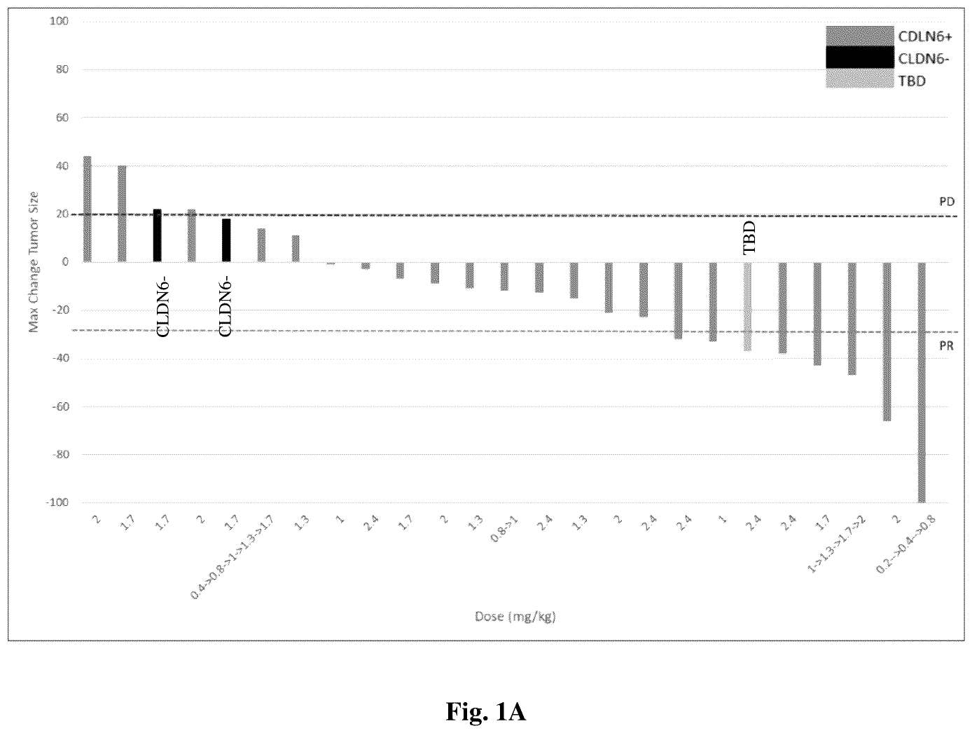

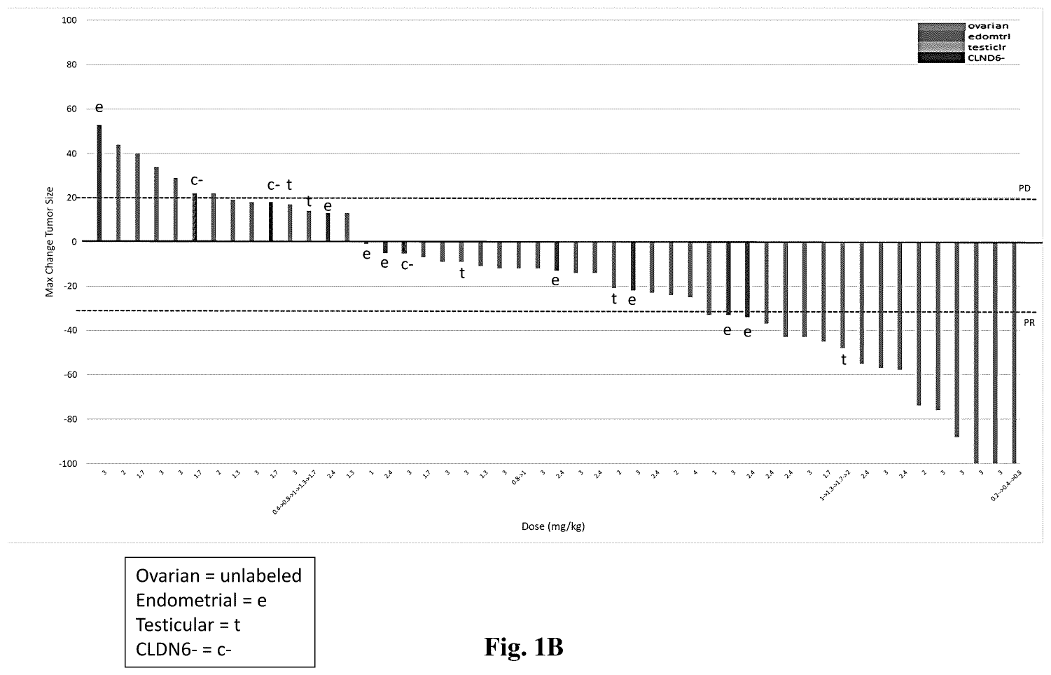

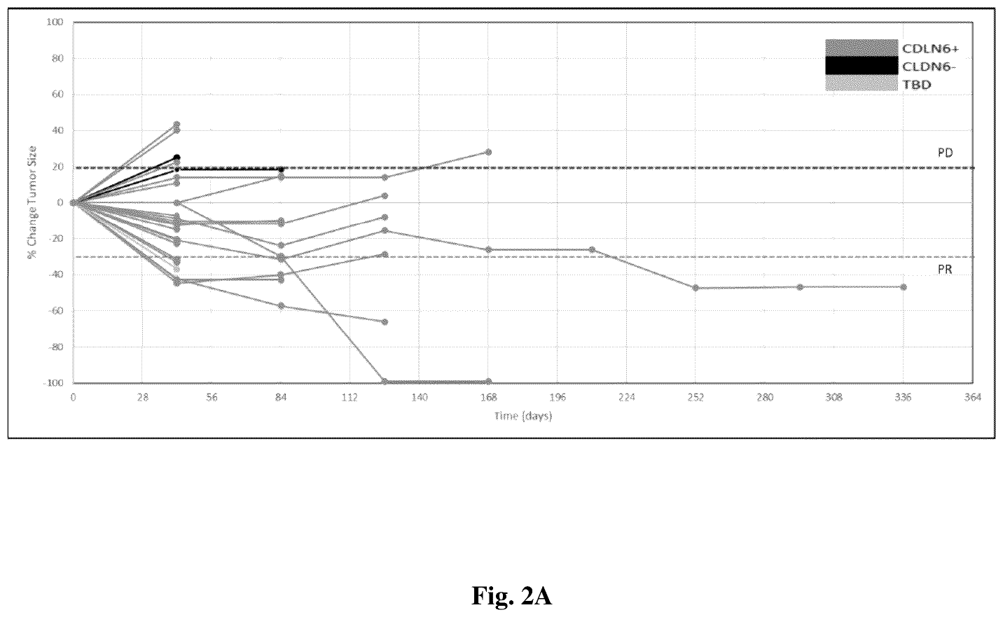

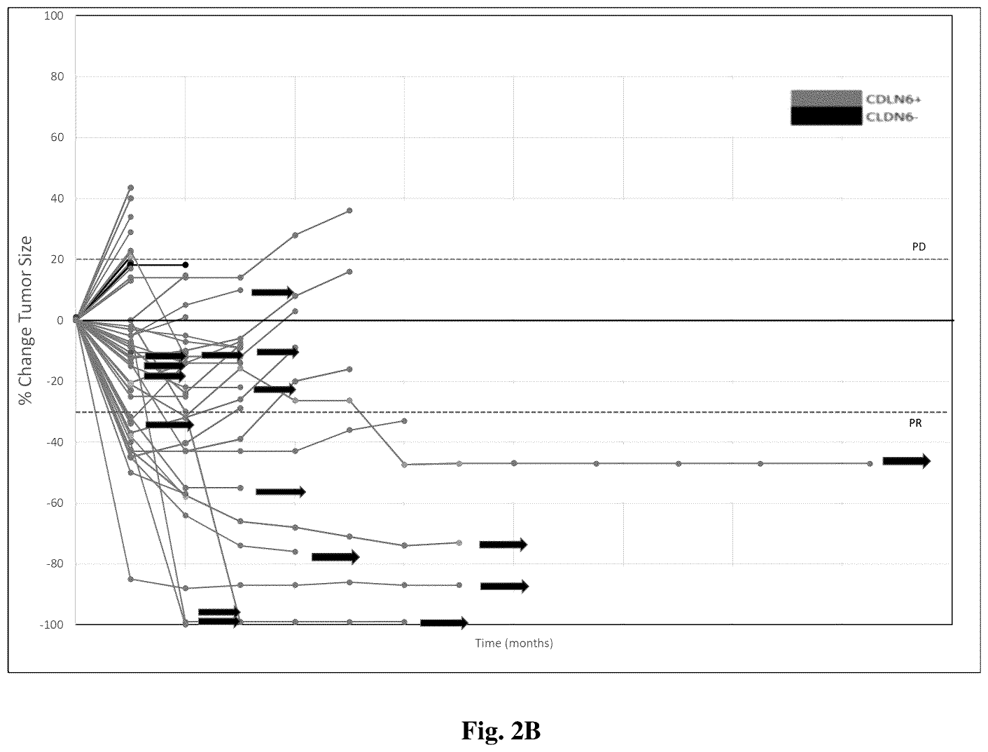

Change in tumor size as a function of dose is shown in FIG. 1 with threshold for disease progression indicated by a dash line labeled as “PD” and partial response indicated by a dash line labeled as “PR.” TBD indicates CLDN6 status to be determined. Change in tumor size over time is shown in FIG. 2.

Blood was collected in all participants for TORL-1-23 serum PK assessment. Pre-dose PK samples were collected within 1 hour prior to infusion on Cycle 1 Day 1 and within 30 minutes prior to all subsequent pre-dose timepoints.

Validated analytical methods were used to analyze serum concentration of TORL-1-23 total antibody (conjugated and unconjugated) and MMAE-conjugated TORL-1-23 antibody and unconjugated (free) circulating MMAE.

Dose-finding is ongoing to identify the maximum tolerated dose (MTD) and doses for evaluation in expansion cohorts of the target populations. Exposure response modeling of available safety and serum MMAE data indicate that doses of up to 6.0 mg/kg may be tolerated.

### Part 2: Recommended Phase 2 Dose Expansion Cohorts

Once doses have been identified in Part 1 as potential RP2D, expansion cohorts of up to 20 participants each will be enrolled at each potential RP2D to characterize the safety, tolerability, and PK at the RP2D in participants with advanced claudin 6 IHC positive ovarian cancer (Cohort 2A), in participants with claudin 6 positive advanced solid cancer (Cohort 2B), and in participants with claudin 6 positive advanced NSCLC (Cohort 2C). In Cohort 2A, participants must have platinum-resistant disease, defined as completing 4 or more cycles of platinum-based therapy and progressing within 6 months of last platinum-based therapy. Participants must have received at least 1 but no more than 4 prior systemic lines of anticancer therapy and for whom single-agent therapy is appropriate at the next line of treatment. In Cohort 2B, participants must have advanced (unresectable) or metastatic solid tumor malignancy that is not responsive to accepted standard therapies or for which there is no standard therapy. In Cohort 2C, advanced (unresectable) or metastatic NSCLC that has progressed on or following treatment with platinum-based regimens and PD1 or PDL1 inhibitor containing regimens and EGFR TKI containing regimen for EGFRmu NSCLC and ALK inhibitor regimen for ALK translocated NSCLC and KRAS G12C inhibitor for KRASG12Cmu NSCLC.

### Example 2

This example describes the preclinical efficacy of a conjugate used in the claimed methods.

CLDN6-23-ADC selectively binds to CLDN6, versus other CLDN family members, inhibits the proliferation of CLDN6+ cancer cells in vitro, and is rapidly internalized in CLDN6+ cells. Robust tumor regressions were observed in multiple CLDN6+ xenograft models and tumor inhibition led to markedly enhanced survival of mice with CLDN6+ PDX tumors or CLDN6+ cancer cells from established human cancer cell lines following treatment with CLDN6-23-ADC.

IHC assessment of cancer tissue microarrays demonstrate elevated levels of CLDN6 in 29% of ovarian epithelial carcinomas. Approximately 45% of high-grade serous ovarian carcinomas and 11% of endometrial carcinomas are positive for the target (for additional preclinical efficacy data of CLDN6-23-ADC, see McDermott et al. (2023) Clin. Cancer Res. 29(11):2131-2143).

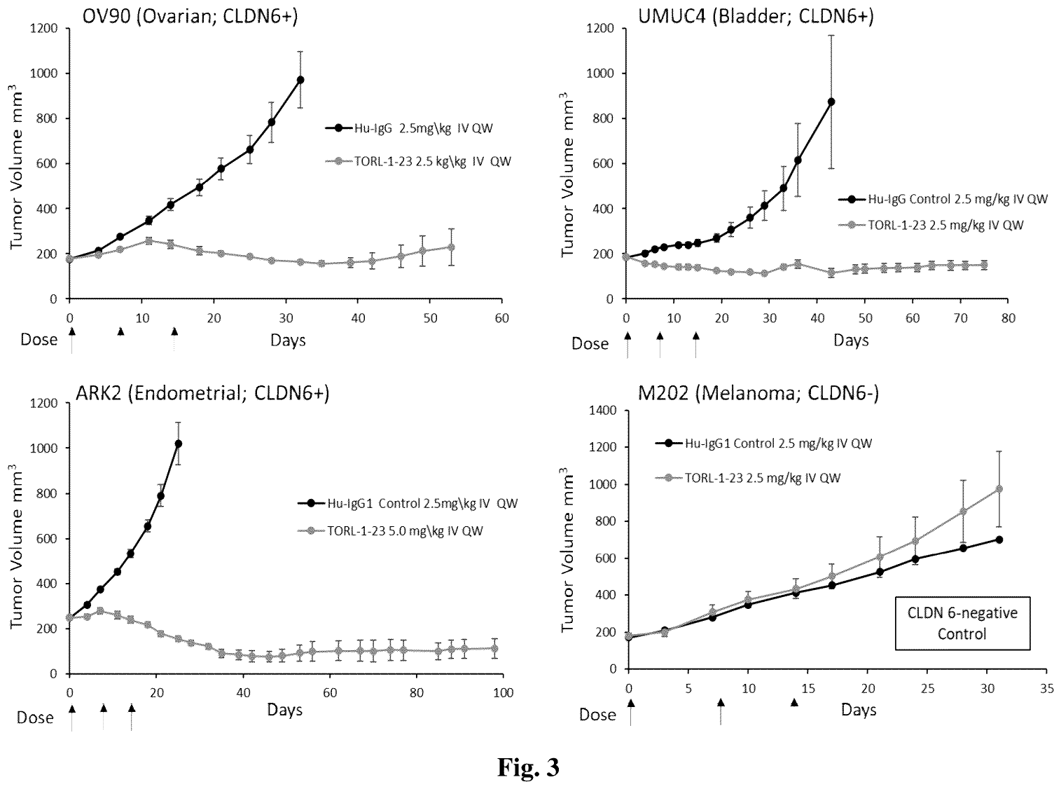

Anti-tumor activity of TORL-1-23 is shown in FIG. 3. Weekly intravenous administration of TORL-1-23 (upward pointing arrow) for three weeks to mice with CLDN6-positive human cancer xenografts from ovarian (OV90) or bladder cancer (UMUC4) at 2.5 mg/kg or endometrial cancer (ARK2) at 5.0 mg/kg resulted in inhibition of tumor growth and reduction in tumor volume that persists substantially beyond the last administered dose (e.g., more than 83 days for ARK2 xenografts in FIG. 3). Specificity of TORL-1-23 anti-tumor activity for CLDN6-positive cancer is demonstrated by a lack of anti-tumor activity on CLDN6-negative human melanoma cancer xenografts (M202) similarly treated with TORL-1-23 at 2.5 mg/kg. Human IgG1 (Hu-IgG1) administered intravenously once a week for three weeks at 2.5 mg/kg served as a negative control.

### Example 3

This example compares the PK properties of conjugates used in the claimed methods to other ADCs having the same linker-heterologous moiety.

An anti-CLDN6 antibody drug conjugate, TORL-1-23, was administered at doses between 0.2 mg/kg to 3.0 mg/kg every 3 weeks to cancer patients participating in phase 1 clinical trial study to determine safety that included a dose-escalation component and cohort expansion to help characterize and guide potential recommended phase 2 dose (RP2D).

Results of the preliminary analyses of the measured PK values are presented herein.

### TORL-1-23 Total Antibody Preliminary Analysis

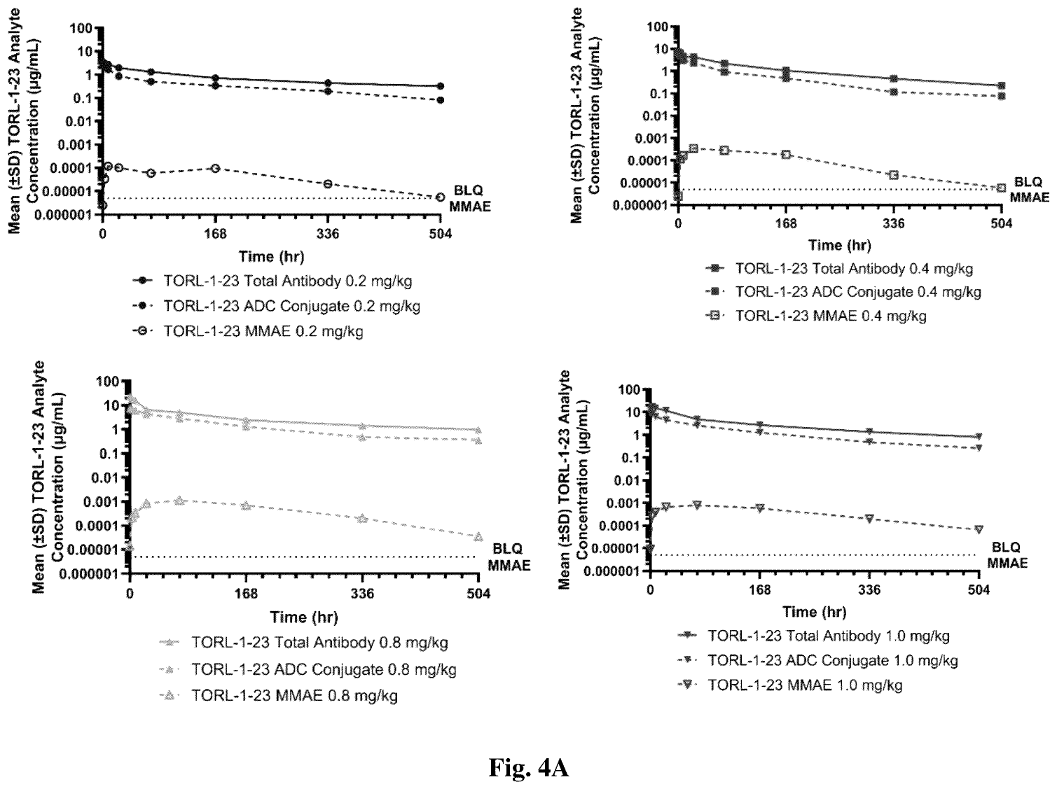

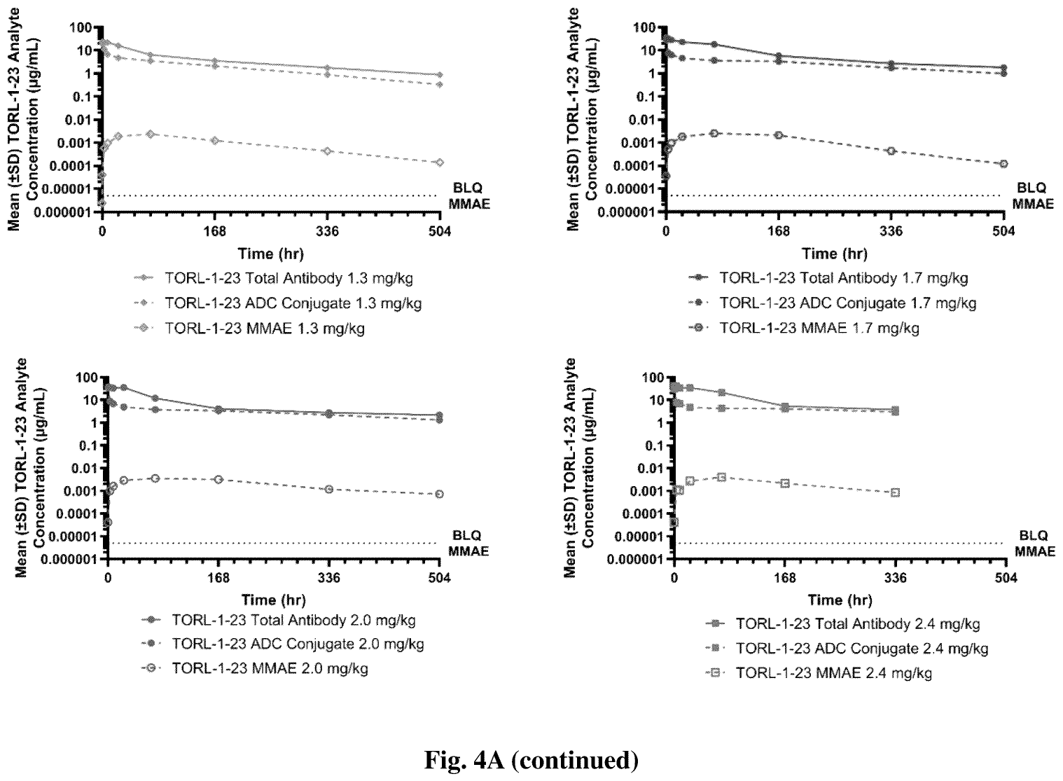

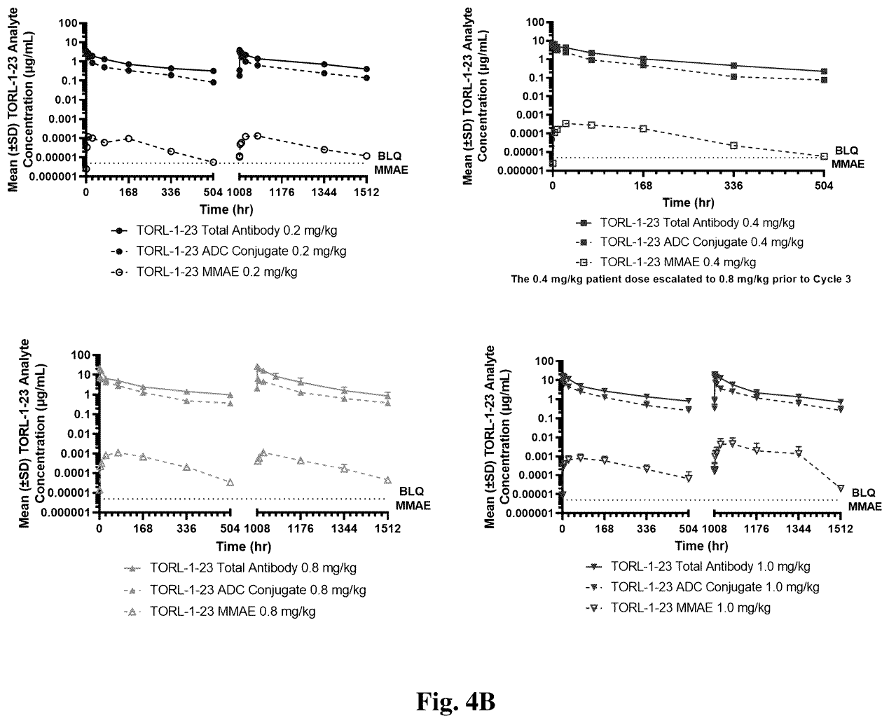

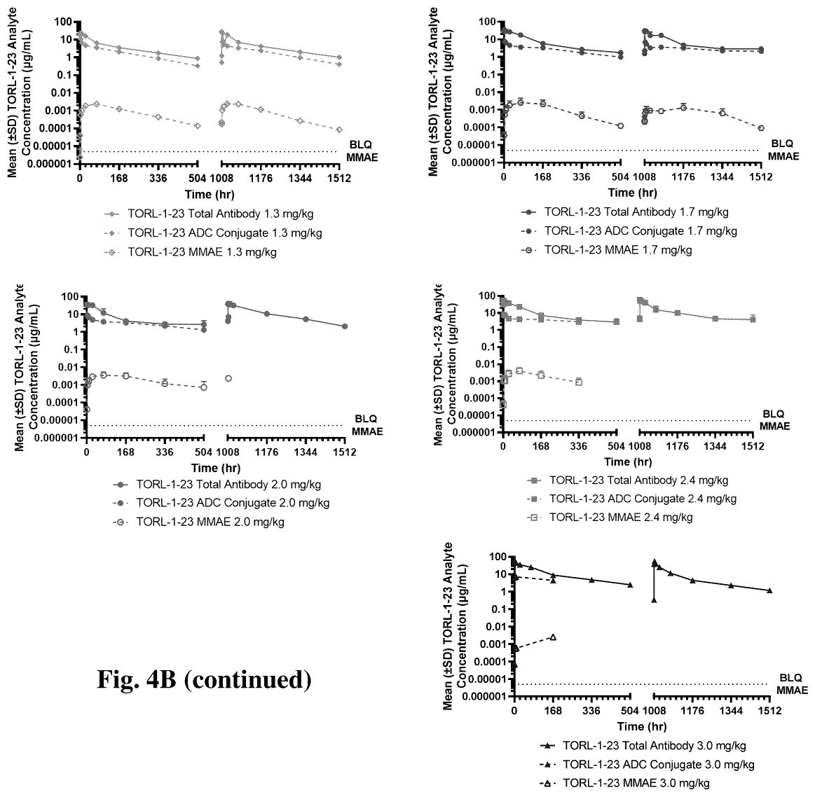

FIG. 4 shows graphs of measured TORL-1-23 total antibody concentration (conjugated and unconjugated), TORL-1-23 concentration, and unconjugated (or free) MMAE following administration of TORL-1-23 preparation to cancer patients participating in the Phase 1 clinical trial and dosed at 0.2 to 2.4 mg/kg (0.2, 0.4, 0.8, 1.0, 1.3, 1.7, 2.0, or 2.4 mg/kg) at cycle 1 (0-504 hrs). The TORL-1-23 is an anti-CLDN6 antibody conjugated to 4 MMAEs per antibody. As can be seen in the graphs, the serum concentrations of TORL-1-23 total antibody (both ADC and free antibody combined) was higher than the serum concentration of TORL-1-23.

Preliminary analyses showed that TORL-1-23 total antibody Cₘₐₓ increased in a nearly dose proportional manner between doses of 0.2 and 3.0 mg/kg. Further, TORL-1-23 total antibody AUC_{0-168 h} and AUC_{0-504 h} increased in a nearly dose proportional manner between doses of 0.2 and 2.4 mg/kg and was slightly less than dose proportional between 2.4 and 3.0 mg/kg.

Minimal to no accumulation in Cₘₐₓ, AUC_{0-168 h}, and AUC_{0-504 h} was observed at doses of 0.2, 1, 1.3, 1.7, 2.0, 2.4, and 3 mg/kg for TORL-1-23 total antibody. However, significant accumulation in AUC_{0-168 h}, but not in Cₘₐₓ or AUC_{0-504 h}, was observed only at a dose of 0.8 mg/kg for a cohort with n=1 in cycle 3.

Preliminary assessments of TORL-1-23 total antibody accumulation based on Cₘₐₓ, AUC_{0-168 h}, and AUC_{0-504 h} values showed in general minimal to no accumulation of total antibody at cycle 3 based on a three-week dosing interval and comparing to cycle 1 values. No significant difference in cycle 3 Cₘₐₓ to cycle 1 Cₘₐₓ ratio was observed for doses between 0.2 and 3.0 mg with the Cₘₐₓ ratios ranging between 0.87 and 1.19. Similarly, when analyzing total antibody accumulation based on AUC_{0-168 h} and AUC_{0-504 h}, minimal to no accumulation in AUC_{0-168 h} and AUC_{0-504 h} were observed at doses of 0.2, 1, 1.3, 1.7, 2.0, 2.4, and 3 mg/kg for TORL-1-23 total antibody at cycle 3 compared to cycle 1. However, for a dose of 0.8 mg/kg and a cohort of n=1, despite significant accumulation in AUC_{0-168 h} observed at cycle 3, no significant accumulation in Cₘₐₓ or AUC_{0-504 h} was observed at a dose of 0.8 mg/kg. Thus, in general, there is no significant accumulation of TORL-1-23 total antibody at a three-week dosing interval for a range of doses from 0.2 to 3 mg/kg every three weeks.

The mean half-life of TORL-1-23 total antibody for cycle 1 ranged from 136 to 290 hours, while the mean half-life of TORL-1-23 total antibody is slightly lower for cycle 3 ranging from 121 to 280 hours.

### TORL-1-23 Preliminary Analysis

Preliminary analysis of the TORL-1-23 concentrations showed that the MMAE-conjugated anti-CLDN6 antibody had a lower serum concentrations than the serum concentrations determined for TORL-1-23 total antibody (see FIG. 4, where the solid line corresponds to total antibody and the dashed line underneath and closest to the solid line corresponds to ADC).

Like TORL-1-23 total antibody, the TORL-1-23 Cₘₐₓ increased in a nearly dose proportional manner between doses of 0.2 and 3.0 mg/kg. TORL-1-23 AUC₀₋₅₀₆ h increased in a nearly dose proportional manner between 0.2 and 2.4 mg/kg. Note that data was only available up to 168-hour post-dose for 3.0 mg/kg (n=1), such that the AUC_{0-504 h} is an extrapolation.

Preliminary assessments of the Cm and AUC_{0-504 h} values at cycle 1 and cycle 3 showed, in general, minimal to no accumulation in Cₘₐₓ or AUC_{0-504 h} at doses of 0.2, 0.8, 1.0, 1.3, and 1.7 mg/kg for TORL-1-23. Data for cycle 3 were not yet available for other dose levels at the time of analysis.

The mean half-life of TORL-1-23 total antibody for cycle 1 ranged from 127 to 387 hours.

### Unconjugated MMAE Preliminary Analysis

Preliminary analysis of unconjugated or free MMAE in the serum showed an increase in a nearly dose proportional manner between doses of 0.2 and 2.4 mg/kg (see FIG. 4, open symbols connected by a dash line). Similarly, the AUC_{0-504 h} of unconjugated MMAE increased in a nearly dose proportional manner between doses of 0.2 and 2.4 mg/kg. Minimal to no accumulation in Cₘₐₓ or AUC_{0-504 h} was observed at doses of 0.2, 0.8, 1.3, and 1.7 mg/kg for the unconjugated or free MMAE. Significant accumulation in Cₘₐₓ and AUC_{0-504 h} was observed at a dose of 1.0 mg/kg.

The mean half-life of the unconjugated or free MMAE for cycle 1 ranged from 68.1 to 131 hours.

Unexpected Advantage of TORL-1-23 Over Other ADCs with MMAE Attached by a Valine-Citrulline Linker

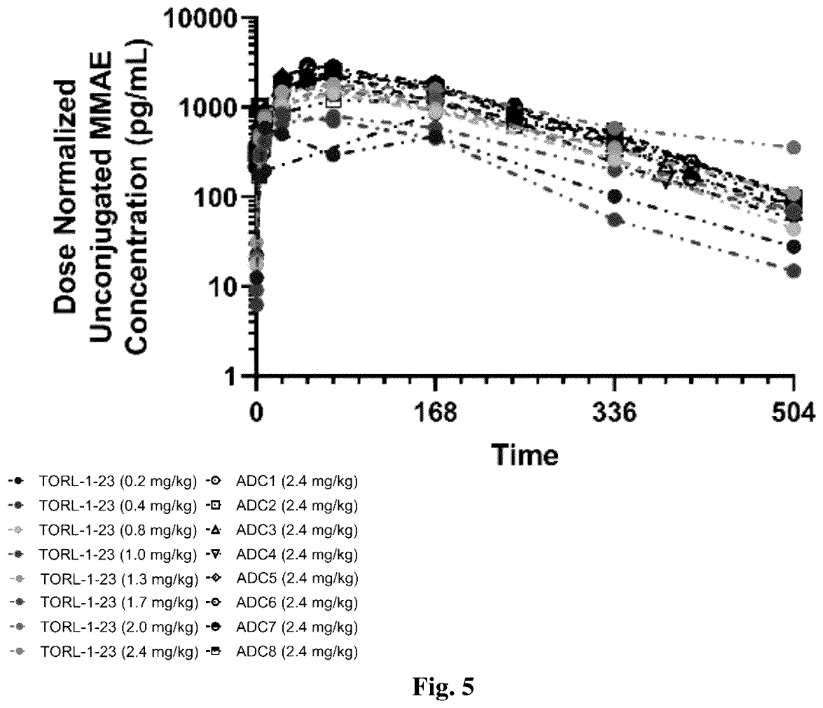

Compared to eight other ADCs, ADC1 to ADC8, which have undergone phase 1 clinical trial in which the therapeutic antibody is a humanized IgG1 ADC conjugated to MMAE through a valine-citrulline linker with 4 MMAE conjugates per antibody (average HAR=˜3.5; Li et al., 2020, MAbs 12(1):1699768), the dose normalized free MMAE concentration following a single dose, systemic exposure to TORL-1-23 is about 3× lower than expected compared to other clinically tested MMAE-containing ADCs, as shown in FIG. 5. Following administration of a single 2.4 mg/kg dose of either TORL-1-23 or eight other ADCs described by Li et al. (2020), the percent MMAE AUC_{inf} (=(MMAE AUC_{inf}/total antibody AUC_{inf})×100) is 0.011% MMAE AUC_{inf} for TORL 1-23 ADC (0.011%), which is lower than those observed for ADC1 to ADC8 described by Li et al. (2020) with the percent MMAE AUC_{inf} ranging from 0.012% to 0.039% (mean of 0.025% for ADC1 to ADC8; median of 0.025% for ADC1 to ADC8). Similar to the finding with the dose normalized free MMAE concentration analysis, the percent MMAE AUC_{inf} following a single 2.4 mg/kg dose ADC administration is about 2.3-fold lower for TORL-1-23 than expected compared to other clinically tested MMAE-containing ADCs. TORL-1-23 provides an unexpected advantage over an average ADC as exemplified from the analysis of eight leading ADCs conjugated to 4 MMAEs through valine-citrulline linkers (Li et al., 2020) in reducing overall exposure to unconjugated MMAE by about 2 to 3-fold. The risk of an adverse event associated with general exposure to circulating unconjugated MMAE is significantly much less than other antibodies similarly conjugated to MMAEs through valine-citrulline linkers.

### INCORPORATION BY REFERENCE

All references, including publications, patent applications, and patents, cited herein are hereby incorporated by reference to the same extent as if each reference were individually and specifically indicated to be incorporated by reference and were set forth in its entirety herein.

### INCORPORATION BY REFERENCE OF MATERIAL SUBMITTED IN THE PRIORITY APPLICATION

McDermott et al. Preclinical Efficacy of the Antibody-Drug Conjugate CLDN6-23-ADC for the Treatment of CLDN6-Positive Solid Tumors. Clin Cancer Res. 2023 Jun. 1; 29(11):2131-2143 (doi: 10.1158/1078-0432.CCR-22-2981. PMID: 36884217; PMCID: PMC10233360), which was filed as Appendix 1 in the U.S. Application No. 63/468,817 to which this application claims benefit of priority, is incorporated herein by reference in its entirety.

### VARIATIONS

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or various language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method for inhibiting a solid tumor expressing claudin-6 (CLDN6) in a human subject, comprising administering to the human subject an effective amount of a composition comprising conjugates of a CLDN6-specific antigen-binding protein covalently bound to heterologous moieties comprising structural formula (I): 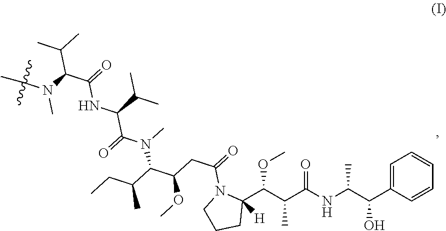 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]
wherein
a first plurality of the conjugates are bound to four heterologous moieties comprising structural formula (I);
at least about 95% of the first plurality of conjugates are structurally homogenous; and
the effective amount is in a range of about
1.7 mg/kg to 6 mg/kg;
2.0 mg/kg to 6 mg/kg; or
2.4 mg/kg to 6 mg/kg.

2-9. (canceled)

10. The method of claim 1, wherein at a Cₘₐₓ after administration, the unbound circulating MMAE in serum is less than about 9 ng/mL, thereby reducing toxicity in the subject and inhibiting the solid tumor.

11. The method of claim 10, wherein the unbound circulating MMAE is less than about 8 ng/mL at Cₘₐₓ.

12. The method of claim 11, wherein the unbound circulating MMAE is less than about 7 ng/mL at Cₘₐₓ.

13. The method of claim 12, wherein the unbound circulating MMAE is less than about 6 ng/mL at Cₘₐₓ.

14-16. (canceled)

17. The method of claim 1, wherein a dose normalized Cₘₐₓ value for the unbound circulating MMAE is less than about 35 pg/mL per mg of the conjugate after administration of the conjugate, thereby reducing toxicity in the subject and inhibiting the solid tumor.

18. The method of claim 17, wherein the dose normalized Cₘₐₓ value for unbound circulating MMAE is less than about 30 pg/mL per mg of the conjugate.

19. The method of claim 18, wherein the dose normalized Cₘₐₓ value for unbound circulating MMAE is less than about 25 pg/mL per mg of the conjugate.

20-22. (canceled)

23. The method of claim 1, wherein at a Cₘₐₓ after first administration of 3.0 mg/kg of the conjugate, the unbound circulating MMAE in serum is about 2.6 ng/mL, thereby reducing toxicity in the subject and inhibiting the solid tumor.

24. The method of claim 1, wherein at a Cₘₐₓ after first administration of 2.4 mg/kg of the conjugate, the unbound circulating MMAE in serum is about 4.1±2.1 ng/mL, thereby reducing toxicity in the subject and inhibiting the solid tumor.

25. The method of claim 1, wherein at a Cₘₐₓ after first administration of 2.0 mg/kg of the conjugate, the unbound circulating MMAE in serum is about 3.7±1.6 ng/mL, thereby reducing toxicity in the subject and inhibiting the solid tumor.

26. The method of claim 1, wherein at a Cₘₐₓ after first administration of 1.7 mg/kg of the conjugate, the unbound circulating MMAE in serum is about 2.7±1.9 ng/mL, thereby reducing toxicity in the subject and inhibiting the solid tumor.

27. The method of claim 1, wherein cycle 3 Cₘₐₓ after three administrations of 1.7 mg/kg of the conjugate, the unbound circulating MMAE in serum is about 1.4±0.8 ng/mL, wherein the conjugate is administered once every three weeks and cycle 3 starts at the 3^{rd} administration of the conjugate, thereby reducing toxicity in the subject and inhibiting the solid tumor.

28. The method of claim 1, wherein the solid tumor is an ovarian tumor.

29. The method of claim 1, wherein the solid tumor is a bladder tumor.

30. The method of claim 1, wherein the solid tumor is a testicular tumor.

31. The method of claim 1, wherein the solid tumor is an endometrial tumor.

32-36. (canceled)

37. The method of claim 1, wherein the effective amount is about 1.7 mg/kg to 5 mg/kg, 1.7 mg/kg to 4 mg/kg, or 1.7 mg/kg to 3 mg/kg.

38. The method of claim 1, wherein the effective amount is about 2.0 mg/kg to 5 mg/kg, 2.0 mg/kg to 4 mg/kg or 2.0 mg/kg to 3 mg/kg.

39. The method of claim 1, wherein the effective amount is about 2.4 mg/kg to 5 mg/kg, 2.4 mg/kg to 4 mg/kg or 2.4 mg/kg to 3 mg/kg.

40-46. (canceled)

47. The method of claim 1, wherein the composition is administered intravenously.

48-53. (canceled)

54. The method of claim 1, wherein the average number of heterologous moieties per CLDN6-specific antigen-binding protein in the composition is about 3.5 to about 4, for example, about 3.6 to about 3.9, preferably about 3.7 to about 3.8.

55. The method of claim 1, wherein, in the first plurality of conjugates, the heterologous moieties are covalently conjugated at unpaired cysteine residues of the CLDN6-specific antigen-binding protein.

56. The method of claim 1, wherein, in the first plurality of conjugates, the heterologous moieties are covalently conjugated at cysteine residues resulting from cleavage of the heavy chain-light chain interchain disulfide bonds of the CLDN6-specific antigen-binding protein.

57. The method of claim 1, wherein about 95%, about 96%, about 97%, or about 98% of the first plurality of conjugates are structurally homogenous.

58. The method of claim 1, wherein the heterologous moiety comprising structural formula (I) has structural formula (II): 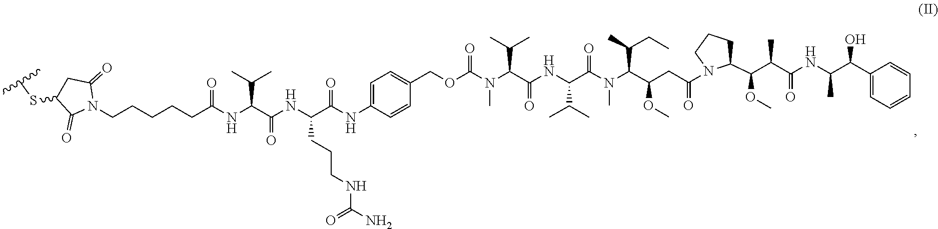 [SMILES: *SC1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C1=O]
wherein 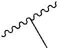
is a covalent thiol bond to the CLDN6-specific antigen-binding protein.

59-65. (canceled)

66. The method of claim 1, wherein the CLDN6-specific antigen-binding protein is selected from a group consisting of:
(a) a heavy chain CDR1 amino acid sequence set forth in Table A or Table A1, or a variant sequence thereof which differs by only one or two amino acids;
(b) a heavy chain CDR2 amino acid sequence set forth in Table A or Table A1, or a variant sequence thereof which differs by only one or two amino acids;
(c) a heavy chain CDR3 amino acid sequence set forth in Table A or Table A1, or a variant sequence thereof which differs by only one or two amino acids;
(d) a light chain CDR1 amino acid sequence set forth in Table A or Table A1, or a variant sequence thereof which differs by only one or two amino acids;
(e) a light chain CDR2 amino acid sequence set forth in Table A or Table A1, or a variant sequence thereof which differs by only one or two amino acids;
(f) a light chain CDR3 amino acid sequence set forth in Table A or Table A1, or a variant sequence thereof which differs by only one or two amino acids; or
a combination of any two or more of (a)-(f).

67. The method of claim 66, wherein the CLDN6-specific antigen-binding protein comprises an antibody that binds CLDN6 or antigen-binding fragment thereof comprising:
(a) the heavy chain (HC) CDR1-3 amino acid sequences of SEQ ID NOs: 23, 24, and 25, and the light chain (LC) CDR1-3 amino acid sequences of SEQ ID NOs: 20, 21, and 22; or
(b) the heavy chain (HC) CDR1-3 amino acid sequences of SEQ ID NOs: 455, 456, and 457, and the light chain (LC) CDR1-3 amino acid sequences of SEQ ID NOs: 476, 477, and 454.

68. The method of claim 1, wherein the CLDN6-specific antigen-binding protein comprises an antibody that binds CLDN6 or antigen-binding fragment thereof comprising:
(i) HC CDR1 having a sequence GFTFSNYW (SEQ ID NO: 23);
(ii) HC CDR2 having a sequence IRLKSDNYAT (SEQ ID NO: 24),
(iii) HC CDR3 having a sequence XDGPPSGX (SEQ ID NO: 457), wherein X at position 1 is N and X at position 8 is S, T, A, C, or Y,
(iv) LC CDR1 having a sequence ENIYSY (SEQ ID NO: 20),
(v) LC CDR2 having a sequence NAK (SEQ ID NO: 21), and
(vi) LC CDR3 having a sequence QHHYTVPWT (SEQ ID NO: 22).

69-70. (canceled)

71. A conjugate of a CLDN6-specific antigen-binding protein covalently bound to heterologous moieties comprising structural formula (I): 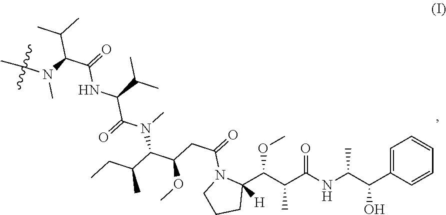 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]
wherein
a first plurality of the conjugates are bound to four heterologous moieties comprising structural formula (I);
at least about 95% of the first plurality of conjugates are structurally homogenous; and
the effective amount is in a range of about
1.7 mg/kg to 6 mg/kg;
2.0 mg/kg to 6 mg/kg; or
2.4 mg/kg to 6 mg/kg for use in treating cancer.


SEQUENCE LISTING (537 sequences)

SEQ ID NO: 1 - protein, 220 aa, Homo sapiens
  REGION 1..220: note = NCBI / NP_067018.2, 2017-06-04
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 2 - protein, 23 aa, Homo sapiens
WTAHAIIRDFYNPLVAEAQKREL

SEQ ID NO: 3 - protein, 13 aa, Homo sapiens
TAHAIIRDFYNPL

SEQ ID NO: 4 - protein, 10 aa, Homo sapiens
LVAEAQKREL

SEQ ID NO: 5 - protein, 220 aa, Homo sapiens
  REGION 1..220: note = NCBI / NP_001297.1, 2017-07-10
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 6 - protein, 209 aa, Homo sapiens
  REGION 1..209: note = NCBI / NP_001296.1, 2017-07-10
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 7 - protein, 217 aa, Homo sapiens
  REGION 1..217: note = NCBI / NP_066192.1, 2017-04-15
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 8 - protein, 7 aa, synthetic construct
  REGION 1..7: note = Synthetic peptide
SSVSSTY

SEQ ID NO: 9 - skipped

SEQ ID NO: 10 - protein, 9 aa, synthetic construct
  REGION 1..9: note = Synthetic peptide
HQYHRSPLT

SEQ ID NO: 11 - protein, 8 aa, synthetic construct
  REGION 1..8: note = Synthetic peptide
GYTFTTYT

SEQ ID NO: 12 - protein, 8 aa, synthetic construct
  REGION 1..8: note = Synthetic peptide
INPSSGYT

SEQ ID NO: 13 - protein, 9 aa, synthetic construct
  REGION 1..9: note = Synthetic peptide
ANGDYYVAY

SEQ ID NO: 14 - protein, 6 aa, synthetic construct
  REGION 1..6: note = Synthetic peptide
ENIYSY

SEQ ID NO: 15 - skipped

SEQ ID NO: 16 - protein, 9 aa, synthetic construct
  REGION 1..9: note = Synthetic peptide
QHHYTVPWT

SEQ ID NO: 17 - protein, 8 aa, synthetic construct
  REGION 1..8: note = Synthetic peptide
GFTFSDYW

SEQ ID NO: 18 - protein, 10 aa, synthetic construct
  REGION 1..10: note = Synthetic peptide
IRLKSDNYAT

SEQ ID NO: 19 - protein, 8 aa, synthetic construct
  REGION 1..8: note = Synthetic peptide
NDGPPSGC

SEQ ID NO: 20 - protein, 6 aa, synthetic construct
  REGION 1..6: note = Synthetic peptide
ENIYSY

SEQ ID NO: 21 - skipped

SEQ ID NO: 22 - protein, 9 aa, synthetic construct
  REGION 1..9: note = Synthetic peptide
QHHYTVPWT

SEQ ID NO: 23 - protein, 8 aa, synthetic construct
  REGION 1..8: note = Synthetic peptide
GFTFSNYW

SEQ ID NO: 24 - protein, 10 aa, synthetic construct
  REGION 1..10: note = Synthetic peptide
IRLKSDNYAT

SEQ ID NO: 25 - protein, 8 aa, synthetic construct
  REGION 1..8: note = Synthetic peptide
NDGPPSGC

SEQ ID NO: 26 - protein, 11 aa, synthetic construct
  REGION 1..11: note = Synthetic peptide
QSLVHSDGNTY

SEQ ID NO: 27 - skipped

SEQ ID NO: 28 - protein, 9 aa, synthetic construct
  REGION 1..9: note = Synthetic peptide
SQSTHVPYT

SEQ ID NO: 29 - protein, 8 aa, synthetic construct
  REGION 1..8: note = Synthetic peptide
GYTFTSYT

SEQ ID NO: 30 - protein, 8 aa, synthetic construct
  REGION 1..8: note = Synthetic peptide
INPSSTYT

SEQ ID NO: 31 - protein, 10 aa, synthetic construct
  REGION 1..10: note = Synthetic peptide
SRGELGGFAY

SEQ ID NO: 32 - protein, 11 aa, synthetic construct
  REGION 1..11: note = Synthetic peptide
QSIVHSNGNTY

SEQ ID NO: 33 - skipped

SEQ ID NO: 34 - protein, 9 aa, synthetic construct
  REGION 1..9: note = Synthetic peptide
FQGSHVPFT

SEQ ID NO: 35 - protein, 8 aa, synthetic construct
  REGION 1..8: note = Synthetic peptide
GYIFTHYI

SEQ ID NO: 36 - protein, 8 aa, synthetic construct
  REGION 1..8: note = Synthetic peptide
INPYNDGT

SEQ ID NO: 37 - protein, 13 aa, synthetic construct
  REGION 1..13: note = Synthetic peptide
ARYYGYPYYSMDY

SEQ ID NO: 38 - protein, 12 aa, synthetic construct
  REGION 1..12: note = Synthetic peptide
QSLLNSRTRKNY

SEQ ID NO: 39 - skipped

SEQ ID NO: 40 - protein, 8 aa, synthetic construct
  REGION 1..8: note = Synthetic peptide
KQSYYLYT

SEQ ID NO: 41 - protein, 9 aa, synthetic construct
  REGION 1..9: note = Synthetic peptide
GYSITSGYY

SEQ ID NO: 42 - protein, 7 aa, synthetic construct
  REGION 1..7: note = Synthetic peptide
ISYDGGI

SEQ ID NO: 43 - protein, 10 aa, synthetic construct
  REGION 1..10: note = Synthetic peptide
ARFGKGAMDY

SEQ ID NO: 44 - protein, 7 aa, synthetic construct
  REGION 1..7: note = Synthetic peptide
SSVSSSY

SEQ ID NO: 45 - skipped

SEQ ID NO: 46 - protein, 9 aa, synthetic construct
  REGION 1..9: note = Synthetic peptide
HQYHRSPPT

SEQ ID NO: 47 - protein, 8 aa, synthetic construct
  REGION 1..8: note = Synthetic peptide
GYSFTGYT

SEQ ID NO: 48 - protein, 8 aa, synthetic construct
  REGION 1..8: note = Synthetic peptide
INPYNGGT

SEQ ID NO: 49 - protein, 12 aa, synthetic construct
  REGION 1..12: note = Synthetic peptide
ARGVYDYDGFTY

SEQ ID NO: 50 - protein, 11 aa, synthetic construct
  REGION 1..11: note = Synthetic peptide
QSLVHSDGNTY

SEQ ID NO: 51 - skipped

SEQ ID NO: 52 - protein, 9 aa, synthetic construct
  REGION 1..9: note = Synthetic peptide
SQSTHVPYT

SEQ ID NO: 53 - protein, 8 aa, synthetic construct
  REGION 1..8: note = Synthetic peptide
GYTFTTYT

SEQ ID NO: 54 - protein, 8 aa, synthetic construct
  REGION 1..8: note = Synthetic peptide
INPRSGYS

SEQ ID NO: 55 - protein, 10 aa, synthetic construct
  REGION 1..10: note = Synthetic peptide
SRGELGGFAY

SEQ ID NO: 56 - protein, 6 aa, synthetic construct
  REGION 1..6: note = Synthetic peptide
QTIGTW

SEQ ID NO: 57 - skipped

SEQ ID NO: 58 - protein, 9 aa, synthetic construct
  REGION 1..9: note = Synthetic peptide
QQLYSIPRT

SEQ ID NO: 59 - protein, 8 aa, synthetic construct
  REGION 1..8: note = Synthetic peptide
GYRFTDYN

SEQ ID NO: 60 - protein, 8 aa, synthetic construct
  REGION 1..8: note = Synthetic peptide
INPNNGGT

SEQ ID NO: 61 - protein, 10 aa, synthetic construct
  REGION 1..10: note = Synthetic peptide
ARDYLYFFDC

SEQ ID NO: 62 - protein, 11 aa, synthetic construct
  REGION 1..11: note = Synthetic peptide
QSLVHSNGNTY

SEQ ID NO: 63 - skipped

SEQ ID NO: 64 - protein, 9 aa, synthetic construct
  REGION 1..9: note = Synthetic peptide
SQITHVPYT

SEQ ID NO: 65 - protein, 8 aa, synthetic construct
  REGION 1..8: note = Synthetic peptide
GYTFTDYS

SEQ ID NO: 66 - protein, 8 aa, synthetic construct
  REGION 1..8: note = Synthetic peptide
ISTETGEP

SEQ ID NO: 67 - protein, 10 aa, synthetic construct
  REGION 1..10: note = Synthetic peptide
TRGLWSSFAY

SEQ ID NO: 68 - protein, 10 aa, synthetic construct
  REGION 1..10: note = Synthetic peptide
KSVSTSGYSY

SEQ ID NO: 69 - skipped

SEQ ID NO: 70 - protein, 9 aa, synthetic construct
  REGION 1..9: note = Synthetic peptide
QHSRELPLT

SEQ ID NO: 71 - protein, 8 aa, synthetic construct
  REGION 1..8: note = Synthetic peptide
GFTFSSFG

SEQ ID NO: 72 - protein, 8 aa, synthetic construct
  REGION 1..8: note = Synthetic peptide
ISSDSRTI

SEQ ID NO: 73 - protein, 11 aa, synthetic construct
  REGION 1..11: note = Synthetic peptide
ARDYGRTYEAY

SEQ ID NO: 74 - protein, 6 aa, synthetic construct
  REGION 1..6: note = Synthetic peptide
QDIGGN

SEQ ID NO: 75 - skipped

SEQ ID NO: 76 - protein, 9 aa, synthetic construct
  REGION 1..9: note = Synthetic peptide
LQRNAYPLT

SEQ ID NO: 77 - protein, 8 aa, synthetic construct
  REGION 1..8: note = Synthetic peptide
GFTFSSYA

SEQ ID NO: 78 - protein, 7 aa, synthetic construct
  REGION 1..7: note = Synthetic peptide
IRSGGTT

SEQ ID NO: 79 - protein, 12 aa, synthetic construct
  REGION 1..12: note = Synthetic peptide
AKVGGNPYPMDY

SEQ ID NO: 80 - protein, 7 aa, synthetic construct
  REGION 1..7: note = Synthetic peptide
SSISSNY

SEQ ID NO: 81 - skipped

SEQ ID NO: 82 - protein, 9 aa, synthetic construct
  REGION 1..9: note = Synthetic peptide
QQGSSIPLT

SEQ ID NO: 83 - protein, 8 aa, synthetic construct
  REGION 1..8: note = Synthetic peptide
GYAFSNYL

SEQ ID NO: 84 - protein, 8 aa, synthetic construct
  REGION 1..8: note = Synthetic peptide
INPGSGGT

SEQ ID NO: 85 - protein, 15 aa, synthetic construct
  REGION 1..15: note = Synthetic peptide
ARSYFGRSYPYTMDY

SEQ ID NO: 86 - protein, 10 aa, synthetic construct
  REGION 1..10: note = Synthetic peptide
QSVDYDGDNY

SEQ ID NO: 87 - skipped

SEQ ID NO: 88 - protein, 9 aa, synthetic construct
  REGION 1..9: note = Synthetic peptide
QQSNEDPFT

SEQ ID NO: 89 - protein, 8 aa, synthetic construct
  REGION 1..8: note = Synthetic peptide
GYTFTDYA

SEQ ID NO: 90 - protein, 8 aa, synthetic construct
  REGION 1..8: note = Synthetic peptide
ISTYSGNT

SEQ ID NO: 91 - protein, 13 aa, synthetic construct
  REGION 1..13: note = Synthetic peptide
ARRGDYSLYAMDY

SEQ ID NO: 92 - protein, 12 aa, synthetic construct
  REGION 1..12: note = Synthetic peptide
QSVLFSSNQKNY

SEQ ID NO: 93 - skipped

SEQ ID NO: 94 - protein, 8 aa, synthetic construct
  REGION 1..8: note = Synthetic peptide
HQYLSSRT

SEQ ID NO: 95 - protein, 8 aa, synthetic construct
  REGION 1..8: note = Synthetic peptide
GFTFSSFG

SEQ ID NO: 96 - protein, 8 aa, synthetic construct
  REGION 1..8: note = Synthetic peptide
ISSDSRTI

SEQ ID NO: 97 - protein, 11 aa, synthetic construct
  REGION 1..11: note = Synthetic peptide
ARDYGRTYEAY

SEQ ID NO: 98 - protein, 10 aa, synthetic construct
  REGION 1..10: note = Synthetic peptide
ESVDNYGISF

SEQ ID NO: 99 - skipped

SEQ ID NO: 100 - protein, 9 aa, synthetic construct
  REGION 1..9: note = Synthetic peptide
QQSKEVPLT

SEQ ID NO: 101 - protein, 8 aa, synthetic construct
  REGION 1..8: note = Synthetic peptide
GFPFSSSA

SEQ ID NO: 102 - protein, 7 aa, synthetic construct
  REGION 1..7: note = Synthetic peptide
INSDGNT

SEQ ID NO: 103 - protein, 13 aa, synthetic construct
  REGION 1..13: note = Synthetic peptide
TRNGDYRYDEFAY

SEQ ID NO: 104 - protein, 7 aa, synthetic construct
  REGION 1..7: note = Synthetic peptide
SSVSSSY

SEQ ID NO: 105 - skipped

SEQ ID NO: 106 - protein, 9 aa, synthetic construct
  REGION 1..9: note = Synthetic peptide
HQYHRSPPT

SEQ ID NO: 107 - protein, 8 aa, synthetic construct
  REGION 1..8: note = Synthetic peptide
GYTFTGYW

SEQ ID NO: 108 - protein, 8 aa, synthetic construct
  REGION 1..8: note = Synthetic peptide
INPSTGYT

SEQ ID NO: 109 - protein, 12 aa, synthetic construct
  REGION 1..12: note = Synthetic peptide
AREGITTVLVDY

SEQ ID NO: 110 - protein, 12 aa, synthetic construct
  REGION 1..12: note = Synthetic peptide
QSVLFSSNQKNY

SEQ ID NO: 111 - skipped

SEQ ID NO: 112 - protein, 8 aa, synthetic construct
  REGION 1..8: note = Synthetic peptide
HQYLSSRT

SEQ ID NO: 113 - protein, 8 aa, synthetic construct
  REGION 1..8: note = Synthetic peptide
GYSFTGYN

SEQ ID NO: 114 - protein, 8 aa, synthetic construct
  REGION 1..8: note = Synthetic peptide
IDPYYGGS

SEQ ID NO: 115 - protein, 14 aa, synthetic construct
  REGION 1..14: note = Synthetic peptide
ARERSGYVFSAMDY

SEQ ID NO: 116 - protein, 12 aa, synthetic construct
  REGION 1..12: note = Synthetic peptide
QSVLFSSNQKNY

SEQ ID NO: 117 - skipped

SEQ ID NO: 118 - protein, 8 aa, synthetic construct
  REGION 1..8: note = Synthetic peptide
HQYLSSRT

SEQ ID NO: 119 - protein, 8 aa, synthetic construct
  REGION 1..8: note = Synthetic peptide
GYSFTGYT

SEQ ID NO: 120 - protein, 8 aa, synthetic construct
  REGION 1..8: note = Synthetic peptide
INPYNGVT

SEQ ID NO: 121 - protein, 16 aa, synthetic construct
  REGION 1..16: note = Synthetic peptide
TRDPLYYGYRDSTMDY

SEQ ID NO: 122 - protein, 11 aa, synthetic construct
  REGION 1..11: note = Synthetic peptide
QSLVHSDGNTY

SEQ ID NO: 123 - skipped

SEQ ID NO: 124 - protein, 9 aa, synthetic construct
  REGION 1..9: note = Synthetic peptide
SQSTHVPYT

SEQ ID NO: 125 - protein, 8 aa, synthetic construct
  REGION 1..8: note = Synthetic peptide
GYTFTSYT

SEQ ID NO: 126 - protein, 8 aa, synthetic construct
  REGION 1..8: note = Synthetic peptide
INPSSTYT

SEQ ID NO: 127 - protein, 10 aa, synthetic construct
  REGION 1..10: note = Synthetic peptide
SRGELGGFAY

SEQ ID NO: 128 - protein, 6 aa, synthetic construct
  REGION 1..6: note = Synthetic peptide
QGIRGN

SEQ ID NO: 129 - skipped

SEQ ID NO: 130 - protein, 9 aa, synthetic construct
  REGION 1..9: note = Synthetic peptide
LQRNAYPLT

SEQ ID NO: 131 - protein, 8 aa, synthetic construct
  REGION 1..8: note = Synthetic peptide
GFTFSSFA

SEQ ID NO: 132 - protein, 7 aa, synthetic construct
  REGION 1..7: note = Synthetic peptide
IRSGGIT

SEQ ID NO: 133 - protein, 13 aa, synthetic construct
  REGION 1..13: note = Synthetic peptide
ARVSTATYYGMDY

SEQ ID NO: 134 - protein, 108 aa, synthetic construct
  REGION 1..108: note = Synthetic peptide
QIVLTQSPAIMSASLGERVTMTCTASSSVSSTYFHWYQQKPGSSPKLWIYSTSNLASGVPRRFSGSASGTSYSLTISSMEAEDAATYYCHQYHRSPLTFGAGTKLELK

SEQ ID NO: 135 - protein, 116 aa, synthetic construct
  REGION 1..116: note = Synthetic peptide
QVQLQQSAAELARPGASVKMSCKASGYTFTTYTMHWVKQRPGQGLEWIGFINPSSGYTDYNQKFKDRTTLTADKSSSTVYMQLSSLTSEDSAVYYCANGDYYVAYWGQGTLVTVSA

SEQ ID NO: 136 - protein, 107 aa, synthetic construct
  REGION 1..107: note = Synthetic peptide
DIQMTQSPASLSASVGETVTITCRISENIYSYLAWYQQKQGKSPQLLVYNAKILVEGVPSRFSGSGSGTQFSLKINSLQPEDFGNYYCQHHYTVPWTFGGGTKLEIK

SEQ ID NO: 137 - protein, 117 aa, synthetic construct
  REGION 1..117: note = Synthetic peptide
EVKLEESGGGLVQPGGSMKLSCVASGFTFSDYWMNWVRQSPEKGLEWVAQIRLKSDNYATHYAESVKGRFTISRDDSKRSVYLQMNNLRAEDTGTYYCNDGPPSGCWGQGTTLIVSS

SEQ ID NO: 138 - protein, 107 aa, synthetic construct; identical to SEQ ID NO: 136
  REGION 1..107: note = Synthetic peptide

SEQ ID NO: 139 - protein, 117 aa, synthetic construct
  REGION 1..117: note = Synthetic peptide
EVKLEESGGGLVQPGGSMKLSCVASGFTFSNYWMNWVRQSPEKGLEWVAQIRLKSDNYATHYAESVKGRFTISRDDSKRSVYLQMNNLRAEDTGTYYCNDGPPSGCWGQGTTLIVSS

SEQ ID NO: 140 - protein, 112 aa, synthetic construct
  REGION 1..112: note = Synthetic peptide
DVVMTQTPLSLPVSLGDQASISCRSSQSLVHSDGNTYLNWYLQKPGQSPKLLIYKVSNRFSGVPDRFSGSGSGTDFTLKIRRVEAEDLGVYFCSQSTHVPYTFGGGTKLEIK

SEQ ID NO: 141 - protein, 117 aa, synthetic construct
  REGION 1..117: note = Synthetic peptide
QVQLQQSGAELARPGASVKMSCKASGYTFTSYTMHWIKQRPGQGQEWIGYINPSSTYTHYIKKFKDKATLTADKSSSTAYMQLRSLTSEDSAVYYCSRGELGGFAYWGQGTLVTVSA

SEQ ID NO: 142 - protein, 112 aa, synthetic construct
  REGION 1..112: note = Synthetic peptide
DVLMTQTPLSLPVSLGDQPSISCRSSQSIVHSNGNTYLDWYLQKPGQSPKLLIYKVSNRFSGVPDRFSGSGSGTDFTLKISRVEAEDLGVYYCFQGSHVPFTFGSGTRLEIK

SEQ ID NO: 143 - protein, 120 aa, synthetic construct
  REGION 1..120: note = Synthetic peptide
EVQLQQSGPELVKPGASVKMSCKASGYIFTHYIMHWVKQKPGQGLEWIGCINPYNDGTKYNEKFKGKATLTSDKSSSTAYMELSSLTSEDSAVYYCARYYGYPYYSMDYWGQGTSVTVSS

SEQ ID NO: 144 - protein, 112 aa, synthetic construct
  REGION 1..112: note = Synthetic peptide
AIVMFQSPSSLVVSAGEKVTMSCKSSQSLLNSRTRKNYLAWYQQKPGQSPKLLIYWASTRESGVPDRFTGSGSGTDFTLTISSVQAEDLAVYYCKQSYYLYTFGGGTKLEIK

SEQ ID NO: 145 - protein, 117 aa, synthetic construct
  REGION 1..117: note = Synthetic peptide
DVQLQESGPGLVKSSQSLSLTCSVTGYSITSGYYWKWIRQFPGNKLEWMGYISYDGGINYNPSLKNRISITRDTSKNQFFLKLNSVTTEDTAKYYCARFGKGAMDYWGQGTSVTVSS

SEQ ID NO: 146 - protein, 108 aa, synthetic construct
  REGION 1..108: note = Synthetic peptide
QIVLTQSPAIMSASLGDRVTMTCTASSSVSSSYLHWYQQKPGSSPKLWIYSTSNLASGVPARFSGSGSGTSYSLTISSMEAEDAATYYCHQYHRSPPTFGSGTKLEIK

SEQ ID NO: 147 - protein, 119 aa, synthetic construct
  REGION 1..119: note = Synthetic peptide
EVQLQQSGPELVKPGASMKISCKASGYSFTGYTMNWVKQSHGKNLEWIGLINPYNGGTNYNQKFKGKATLTVDKSSSTAYMELLSLTSEDSAVYYCARGVYDYDGFTYWGQGTLVTVSA

SEQ ID NO: 148 - protein, 112 aa, synthetic construct
  REGION 1..112: note = Synthetic peptide
DVVMTQTPLSLPVSLGDQASISCRSSQSLVHSDGNTYLYWYLQKPGQSPKLLIYKVSNRFSGVPDRFSGSGSGTDFTLKISRVEAEDLGVYFCSQSTHVPYTFGGGTKLEIK

SEQ ID NO: 149 - protein, 117 aa, synthetic construct
  REGION 1..117: note = Synthetic peptide
QVQLQQSGAELARPGASVKMSCKASGYTFTTYTMHWLKQRPGQGLEWIGYINPRSGYSNYNQKFKDKATLTADKSSNTAYMQLNTLTSEDSKVYYCSRGELGGFAYWGQGTLVTVSA

SEQ ID NO: 150 - protein, 107 aa, synthetic construct
  REGION 1..107: note = Synthetic peptide
DIQMTQSPASQSASLGESVTITCLASQTIGTWLAWYQQKPGKSPQLLIYAAASLADGVPSRFSGSGSGTRFSFKISSLQAEDFVSYYCQQLYSIPRTFGGGTKLEIK

SEQ ID NO: 151 - protein, 117 aa, synthetic construct
  REGION 1..117: note = Synthetic peptide
EVQLQQSGPELVKPGASVKMSCKASGYRFTDYNMHWVKQSHGKSLEWIGYINPNNGGTNYNQNFKGKATLTVNKSSSTAYMELRSLTSEDSAAYYCARDYLYFFDCWGQGTTLTVSS

SEQ ID NO: 152 - protein, 112 aa, synthetic construct
  REGION 1..112: note = Synthetic peptide
DVVMTQTPLSLPVSLGDQASISCRASQSLVHSNGNTYLHWFLQKPGQSPKLLIYKVSNRFSGVPDRFSGSGSRTDFTLKISRVEAEDLGVYFCSQITHVPYTFGGGTKLEIK

SEQ ID NO: 153 - protein, 117 aa, synthetic construct
  REGION 1..117: note = Synthetic peptide
QIQLVQSGPALKKPGETVKISCKASGYTFTDYSMHWIKQAPGKGLKWMGWISTETGEPTYADGFKGRFDFSLETSADTAYLSINNLTNEDTATYFCTRGLWSSFAYWGQGTLVTVSA

SEQ ID NO: 154 - protein, 111 aa, synthetic construct
  REGION 1..111: note = Synthetic peptide
DIVLTQSPASLAVSLGQRATISCRASKSVSTSGYSYIHWYQQKPGQPPKLLIYLASNLESGVPARFSGSGSGTDFTLNIHPVEEEDAATYYCQHSRELPLTFGAGTKLELK

SEQ ID NO: 155 - protein, 118 aa, synthetic construct
  REGION 1..118: note = Synthetic peptide
DVQLVESGGGLVQPRGSRKLSCAASGFTFSSFGMHWVRQAPEKGLEWVAYISSDSRTIYYADTVKGRFTISRDNPTNTLFLQMTSLRSEDTAMYYCARDYGRTYEAYWGQGTLVTVSA

SEQ ID NO: 156 - protein, 107 aa, synthetic construct
  REGION 1..107: note = Synthetic peptide
DIQMIQSPSSMFASLGDRVSLSCRASQDIGGNLDWYQQKPGGTIKLLIYSTSNLNSGVPSRFSGSGSGSDYSLTITSLESEDFADYYCLQRNAYPLTFGAGTKLELK

SEQ ID NO: 157 - protein, 118 aa, synthetic construct
  REGION 1..118: note = Synthetic peptide
EVKLMESGGDLVKPGGSLKLSCAASGFTFSSYAMSWVRQTPEKRLEWVASIRSGGTTYYPDSVKGRFTISRDNARNILYLRMSSLRSEDTAIYYCAKVGGNPYPMDYWGQGTSVTVSS

SEQ ID NO: 158 - protein, 108 aa, synthetic construct
  REGION 1..108: note = Synthetic peptide
EIVLTQSPTTMAASPGEKITITCSASSSISSNYLHWYQQKPGFSPKLLIYRTSNLASGVPARFSGSGSGTSYSLTIGTMEAEDVATYYCQQGSSIPLTFGAGTKLELK

SEQ ID NO: 159 - protein, 122 aa, synthetic construct
  REGION 1..122: note = Synthetic peptide
QVQLQQSGAELVRPGTSVKVSCKASGYAFSNYLIEWVKQRPGQGLEWIGVINPGSGGTNYNEKFKGKATMTADKSSSTAYMHLSNLTSEDSVVYFCARSYFGRSYPYTMDYWGQGTSVTVSS

SEQ ID NO: 160 - protein, 111 aa, synthetic construct
  REGION 1..111: note = Synthetic peptide
DIVLTQSPASLAVSLGQRATISCKASQSVDYDGDNYVNWYQQKVGQPPKLLISAASNLESGIPARFSGSGSGTDFTLNIHPVEEEDAATYYCQQSNEDPFTFGSGTKLEIK

SEQ ID NO: 161 - protein, 120 aa, synthetic construct
  REGION 1..120: note = Synthetic peptide
QVQLQQSGPELVRPGVSVKISCKGSGYTFTDYAMHWVKQSHAKSLEWIGVISTYSGNTNYNQKFQDKATMTVDKSSSTAYMALARLTSDDSAIYYCARRGDYSLYAMDYWGQGTSVTVSS

SEQ ID NO: 162 - protein, 112 aa, synthetic construct
  REGION 1..112: note = Synthetic peptide
NIMMTQSPSSLAVSAGEKVTMSCKSSQSVLFSSNQKNYLAWYQQKPGQSPRLLIYWASTRESGVPDRFTGSGSGTDFTLTISNVQAEDLAVYYCHQYLSSRTFGAGTKLELK

SEQ ID NO: 163 - protein, 118 aa, synthetic construct; identical to SEQ ID NO: 155
  REGION 1..118: note = Synthetic peptide

SEQ ID NO: 164 - protein, 111 aa, synthetic construct
  REGION 1..111: note = Synthetic peptide
DIVLTQSPASLALSLGQRATISCRASESVDNYGISFMNWFQQKPGQPPKLLIYAASNQGSGVPARFSGSGSGTDFSLNIHPMEEDDTAMYFCQQSKEVPLTFGAGTKLELK

SEQ ID NO: 165 - protein, 119 aa, synthetic construct
  REGION 1..119: note = Synthetic peptide
EVRLVESGGGLMQPGGSLKLPCAASGFPFSSSAMSWVRQTPEKRLEWVASINSDGNTYYPDSVKGRFTISRDSARNILYLQMSSLRSEDTAMYYCTRNGDYRYDEFAYWGQGTLVTVSA

SEQ ID NO: 166 - protein, 108 aa, synthetic construct
  REGION 1..108: note = Synthetic peptide
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELK

SEQ ID NO: 167 - protein, 119 aa, synthetic construct
  REGION 1..119: note = Synthetic peptide
QVQLQQSGAELAKPGASVKMSCKASGYTFTGYWMHWVKQRPGQGLEWLGYINPSTGYTESNQKFKDKATLTADKSSTTAYMQLRSLTPEDSAVYYCAREGITTVLVDYWGQGTTLTVSS

SEQ ID NO: 168 - protein, 112 aa, synthetic construct; identical to SEQ ID NO: 162
  REGION 1..112: note = Synthetic peptide

SEQ ID NO: 169 - protein, 121 aa, synthetic construct
  REGION 1..121: note = Synthetic peptide
QVQLKQSGPELEKPGASVKISCKASGYSFTGYNMNWVKQSNGKSLEWIGNIDPYYGGSTYNQKFTGKATLTVDKSSSTAYMQLKSLTSEDSAVYYCARERSGYVFSAMDYWGQGTSVTVSS

SEQ ID NO: 170 - protein, 112 aa, synthetic construct; identical to SEQ ID NO: 162
  REGION 1..112: note = Synthetic peptide

SEQ ID NO: 171 - protein, 123 aa, synthetic construct
  REGION 1..123: note = Synthetic peptide
EVQLQQSGPELVKPGGSMKISCKASGYSFTGYTMNWVKRSHGKNLEWIGLINPYNGVTTYNQNFKGKATLAVDKSSSTAYMELLGLTSEDSAVYYCTRDPLYYGYRDSTMDYWGQGTSVTVSS

SEQ ID NO: 172 - protein, 112 aa, synthetic construct; identical to SEQ ID NO: 140
  REGION 1..112: note = Synthetic peptide

SEQ ID NO: 173 - protein, 117 aa, synthetic construct
  REGION 1..117: note = Synthetic peptide
EVQLQQSGAELARPGASVKMSCKASGYTFTSYTMHWIKQRPGQGQEWIGYINPSSTYTHYIKKFKDKATLTADKSSSTAYMQLRSLTSEDSAVYYCSRGELGGFAYWGQGTLVTVSA

SEQ ID NO: 174 - protein, 107 aa, synthetic construct
  REGION 1..107: note = Synthetic peptide
DIQMIQSPSSMFASLGDRVSLSCRASQGIRGNLDWYQQKPGGTIKLLIYSTSILNSGVPSRFSGSGSGSDYSLTITSLESEDFADYYCLQRNAYPLTFGSGTKLELK

SEQ ID NO: 175 - protein, 119 aa, synthetic construct
  REGION 1..119: note = Synthetic peptide
EVKLVESGGGLMKPGGSLKLSCAASGFTFSSFALSWVRQTPEKRLEWVASIRSGGITYHADSVKGRFTISRDNAGNILYLQMNSLRSEDTAMYFCARVSTATYYGMDYWGQGTSVTVSS

SEQ ID NO: 176 - protein, 219 aa, Mus musculus
  REGION 1..219: note = NCBI / NP_061247.1, 2017-08-07
MASTGLQILGIVLTLLGWVNALVSCALPMWKVTAFIGNSIVVAQMVWEGLWMSCVVQSTGQMQCKVYDSLLALPQDLQAARALCVVTLLIVLLGLLVYLAGAKCTTCVEDRNSKSRLVLISGIIFVISGVLTLIPVCWTAHSIIQDFYNPLVADAQKRELGASLYLGWAASGLLLLGGGLLCCACSSGGTQGPRHYMACYSTSVPHSRGPSEYPTKNYV

SEQ ID NO: 177 - protein, 80 aa, synthetic construct
  REGION 1..80: note = Synthetic peptide
MASAGMQILGVVLTLLGWVNGLVSCALPMWKVTAFIGNSIVVAQVVWEGLWMSCVVQSTGQMQCKVYDSLLALPQDLQAA

SEQ ID NO: 178 - protein, 220 aa, synthetic construct
  REGION 1..220: note = Synthetic peptide
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAVIRDFYNPLVAEAQKRELGASLYLGWAASGLLLLGGGLLCCTCPSGGSQGPSHYMARYSTSAPAISRGPSEYPTKNYV

SEQ ID NO: 179 - protein, 322 aa, synthetic construct
  REGION 1..322: note = Synthetic peptide
CAGATCGTGCTGACTCAGAGTCCTTCAATTATGTCCGTGAGCCCAGGCGAGAAGGTCACCATCACATGCAGTGCCTCCAGCTCTGTCTCATACATGCACTGGTTCCAGCAGAAGCCAGGGACCAGTCCCAAGCTGTGCATCTACTCTACATCGAACCTGGCCTCCGGAGTGCCCGCAAGGTTTAGCGGTCGGGGCTCTGGAACTTCATACTCCCTGACCATCTCGCGGGTGGCCGCTGAGGATGCAGCAACATACTATTGCCAGCAGAGGTCCAATTATCCCCCTTGGACATTCGGCGGAGGTACCAAACTCGAGATTAAGC

SEQ ID NO: 180 - protein, 351 aa, synthetic construct
  REGION 1..351: note = Synthetic peptide
GAAGTCCAGCTGCAGCAGTCTGGCCCTGAACTGGTGAAGCCTGGCGCCAGCATGAAGATCTCCTGCAAGGCCAGCGGCTACTCCTTCACCGGCTATACAATGAACTGGGTGAAGCAGTCCCACGGCAAGAATCTGGAGTGGATCGGCCTGATCAACCCATACAATGGCGGCACCATCTACAACCAGAAGTTTAAGGGCAAGGCCACCCTGACAGTGGACAAGAGCTCCTCTACCGCCTACATGGAGCTGCTGTCTCTGACAAGCGAGGACTCCGCCGTGTACTATTGCGCCCGGGACTACGGCTTCGTGCTGGACTATTGGGGCCAGGGCACCACACTGACAGTGAGCTCC

SEQ ID NO: 181 - protein, 107 aa, synthetic construct
  REGION 1..107: note = Synthetic peptide
QIVLTQSPSIMSVSPGEKVTITCSASSSVSYMHWFQQKPGTSPKLCIYSTSNLASGVPARFSGRGSGTSYSLTISRVAAEDAATYYCQQRSNYPPWTFGGGTKLEIK

SEQ ID NO: 182 - protein, 117 aa, synthetic construct
  REGION 1..117: note = Synthetic peptide
EVQLQQSGPELVKPGASMKISCKASGYSFTGYTMNWVKQSHGKNLEWIGLINPYNGGTIYNQKFKGKATLTVDKSSSTAYMELLSLTSEDSAVYYCARDYGFVLDYWGQGTTLTVSS

SEQ ID NO: 183 - DNA, 324 bp, synthetic construct
  misc_feature 1..324: note = Synthetic polynucleotide
  source 1..324: mol_type = other DNA
gaaattgtgctcacccagtctccagcactcatggctgcatctccaggggagaaggtcaccatcacctgcagtgtcagctcaagtataagttccagcaacttgcactggtaccagcagaagtcaggaacctcccccaaactctggatttatggcacatccaacctggcttctggagtccctgttcgcttcagtggcagtggatctgggacctcttattctctcacaatcagcaacatggaggctgaagatgctgccacttattactgtcaacagtggagtagttacccacacacgttcggaggggggaccaagctggaaataaaa

SEQ ID NO: 184 - DNA, 378 bp, synthetic construct
  misc_feature 1..378: note = Synthetic polynucleotide
  source 1..378: mol_type = other DNA
caggtccaaatgcagcagtctggagctgagctggtaaggcctgggacttcagtgaaggtgtcctgcaaggcttctggatacgccttcactaattacttgatagagtgggtaaagcagaggcctggacagggccttgagtggattggactgattaatcctggaagtggtggtactaattacaatgagaagttcaagggcaaggcaacactgactgcagacaaatcctccaccactgcctacatgcagctcagcagcctgacatctgatgactctgcggtttatttctgtgcaagacggtcccctctagggagttggatctactatgcttacgacggtgttgcttactggggccaagggactctggtcactgtctctgca

SEQ ID NO: 185 - protein, 108 aa, synthetic construct
  REGION 1..108: note = Synthetic peptide
EIVLTQSPALMAASPGEKVTITCSVSSSISSSNLHWYQQKSGTSPKLWIYGTSNLASGVPVRFSGSGSGTSYSLTISNMEAEDAATYYCQQWSSYPHTFGGGTKLEIK

SEQ ID NO: 186 - protein, 126 aa, synthetic construct
  REGION 1..126: note = Synthetic peptide
QVQMQQSGAELVRPGTSVKVSCKASGYAFTNYLIEWVKQRPGQGLEWIGLINPGSGGTNYNEKFKGKATLTADKSSTTAYMQLSSLTSDDSAVYFCARRSPLGSWIYYAYDGVAYWGQGTLVTVSA

SEQ ID NO: 187 - DNA, 645 bp, Mus musculus
  source 1..645: mol_type = genomic DNA
gatattgtgctaactcagtctccagccaccctgtctgtgactccaggaaatagcgtcagtctttcctgcagggccagccaaagtattggcggtaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacagagtaacagctggccttacacgttcggaggggggaccaagctggaaataaaacgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 188 - DNA, 1335 bp, Mus musculus
  source 1..1335: mol_type = genomic DNA
gacgtgcagcttcaggagtcaggacctagcctcgtgaaaccttctcagactctgtccctcacctgttctgtcactggcgactccatcaccagtgattactggagctggatccggaaattcccagggaatagacttgagtacatggggtacgtaagctacagtggtagcacttactacaatccatctctcaaaagtcgaatctccatcacccgagacacatccaagaaccagtactacctggatttgaattctgtgactactgaggacacagccacatattactgtgcaaactgggacggtgattactggggccaagggactctggtcactgtctcttcagcagctaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 189 - DNA, 8806 bp, synthetic construct
  misc_feature 1..8806: note = Synthetic peptide
  source 1..8806: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttccgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaactagtggatcccccgggctgcaggaattcgtcgactggatccggtaccgaggagatctgccgccgcgatcgccggcgcgccagatctcaagcttaactagttagcggaccgacgcgtacgcggccgctcgagatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtccggactcagataagtttaaacccgatatcatcatctagggcggccaattccgcccctctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaaattagatcgataccgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaatacgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 190 - protein, 220 aa, synthetic construct
  REGION 1..220: note = Synthetic peptide
  VARIANT 143: note = Xaa can be any naturally occurring amino acid
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAXIRDFYNPLVAEAQKRELGASLYLGWAASGLLLLGGGLLCCTCPSGGSQGPSHYMARYSTSAPAISRGPSEYPTKNYV

SEQ ID NO: 191 - DNA, 68 bp, synthetic construct
  misc_feature 1..68: note = Synthetic polynucleotide
  source 1..68: mol_type = other DNA
cagaaactcatctcagaagaggatctggcagcaaatgatatcctggattacaaggatgacgacgataa

SEQ ID NO: 192 - DNA, 683 bp, Homo sapiens
  misc_feature 1..683: note = NCBI / NM_021195.4, 2017-06-04,
  source 1..683: mol_type = genomic DNA
ccgcgatcgccatggcctctgccggaatgcagatcctgggagtcgtcctgacactgctgggctgggtgaatggcctggtctcctgtgccctgcccatgtggaaggtgaccgctttcatcggcaacagcatcgtggtggcccaggtggtgtgggagggcctgtggatgtcctgcgtggtgcagagcaccggccagatgcagtgcaaggtgtacgactcactgctggcgctgccacaggacctgcaggctgcacgtgccctctgtgtcatcgccctccttgtggccctgttcggcttgctggtctaccttgctggggccaagtgtaccacctgtgtggaggagaaggattccaaggcccgcctggtgctcacctctgggattgtctttgtcatctcaggggtcctgacgctaatccccgtgtgctggacggcgcatgccgtcatccgggacttctataaccccctggtggctgaggcccaaaagcgggagctgggggcctccctctacttgggctgggcggcctcaggccttttgttgctgggtggggggttgctgtgctgcacttgcccctcgggggggtcccagggccccagccattacatggcccgctactcaacatctgcccctgccatctctcgggggccctctgagtaccctaccaagaattacgtcacgcgtacgcgg

SEQ ID NO: 193 - DNA, 677 bp, Mus musculus
  misc_feature 1..677: note = NCBI / NM_018777.4, 2017-08-07
  source 1..677: mol_type = genomic DNA
gccgcgatcgccatggcctctactggtctgcaaatcttggggatcgtcctgaccctgcttggctgggtcaacgccctggtgtcctgtgccctgcccatgtggaaggtgaccgccttcatcggcaacagcatcgtcgtggcccagatggtgtgggaggggctgtggatgtcctgtgtggttcagagcactggccagatgcagtgcaaggtgtatgactcactgttggcgctgccccaggacctgcaggctgccagagccctctgtgttgtcaccctcctcattgtcctgcttggcctgctcgtgtacctggctggagccaagtgcactacctgtgtggaagataggaactccaagtctcgtctggtgctcatctctggcatcatctttgtcatttctggggtcctgacgctcattcctgtctgctggactgcccactctatcatccaggacttctacaaccccttggtggctgatgctcaaaagcgggagctgggggcctccctctacctgggctgggcagcctcaggccttttgctgctgggtggagggctactatgctgcgcctgctcttctggagggacccagggacccagacattacatggcctgctattctacatctgtcccacattctcggggaccctccgaatatcccaccaagaattatgtgacgcgtac

SEQ ID NO: 194 - DNA, 683 bp, synthetic construct
  misc_feature 1..683: note = Synthetic polynucleotide
  source 1..683: mol_type = other DNA
cgcgatcgccatggcctctgccggaatgcagatcctgggagtcgtcctgacactgctgggctgggtgaatggcctggtctcctgtgccctgcccatgtggaaggtgaccgctttcatcggcaacagcatcgtggtggcccaggtggtgtgggagggcctgtggatgtcctgcgtggtgcagagcaccggccagatgcagtgcaaggtgtacgactcactgctggcgctgccacaggacctgcaggctgcacgtgccctctgtgtcatcgccctccttgtggccctgttcggcttgctggtctaccttgctggggccaagtgtaccacctgtgtggaggagaaggattccaaggcccgcctggtgctcacctctgggattgtctttgtcatctcaggggtcctgacgctaatccccgtgtgctggacggcgcatgccatcatccgggacttctataaccccctggtggctgaggcccaaaagcgggagctgggggcctccctctacttgggctgggcggcctcaggccttttgttgctgggtggggggttgctgtgctgcacttgcccctcgggggggtcccagggccccagccattacatggcccgctactcaacatctgcccctgccatctctcgggggccctctgagtaccctaccaagaattacgtcacgcgtacgcggc

SEQ ID NO: 195 - DNA, 672 bp, Homo sapiens
  misc_feature 1..672: note = NCBI / NM_020982.3, 2017-04-15
  source 1..672: mol_type = genomic DNA
gccgcgatcgccatggcttcgaccggcttagaactgctgggcatgaccctggctgtgctgggctggctggggaccctggtgtcctgcgccctgcccctgtggaaggtgaccgccttcatcggcaacagcatcgtggtggcccaggtggtgtgggagggcctgtggatgtcctgcgtggtgcagagcacgggccagatgcagtgcaaggtgtacgactcactgctggctctgccgcaggacctgcaggccgcacgtgccctctgtgtcattgccctcctgctggccctgcttggcctcctggtggccatcacaggtgcccagtgtaccacgtgtgtggaggacgaaggtgccaaggcccgtatcgtgctcaccgcgggggtcatcctcctcctcgccggcatcctggtgctcatccctgtgtgctggacggcgcacgccatcatccaggacttctacaaccccctggtggctgaggccctcaagcgggagctgggggcctccctctacctgggctgggcggcggctgcactgcttatgctgggcggggggctcctctgctgcacgtgccccccgccccaggtcgagcggccccgcggacctcggctgggctactccatcccctcccgctcgggtgcatctggactggacaagagggactacgtgacgcgtacg

SEQ ID NO: 196 - DNA, 651 bp, Homo sapiens
  misc_feature 1..651: note = NCBI / NM_001305.4, 2017-07-10
  source 1..651: mol_type = genomic DNA
ccgcgatcgccatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgactcgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcgccaaggccaagaccatgatcgtggcgggcgtggtgttcctgttggccggccttatggtgatagtgccggtgtcctggacggcccacaacatcatccaagacttctacaatccgctggtggcctccgggcagaagcgggagatgggtgcctcgctctacgtcggctgggccgcctccggcctgctgctccttggcggggggctgctttgctgcaactgtccaccccgcacagacaagccttactccgccaagtattctgctgcccgctctgctgctgccagcaactacgtgacgcgtacgcggc

SEQ ID NO: 197 - DNA, 680 bp, Homo sapiens
  misc_feature 1..680: note = NCBI / NM_001306.3, 2017-07-10
  source 1..680: mol_type = genomic DNA
gccgcgatcgccatgtccatgggcctggagatcacgggcaccgcgctggccgtgctgggctggctgggcaccatcgtgtgctgcgcgttgcccatgtggcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccgcgccctcatcgtggtggccatcctgctggccgccttcgggctgctagtggcgctggtgggcgcccagtgcaccaactgcgtgcaggacgacacggccaaggccaagatcaccatcgtggcaggcgtgctgttccttctcgccgccctgctcaccctcgtgccggtgtcctggtcggccaacaccattatccgggacttctacaaccccgtggtgcccgaggcgcagaagcgcgagatgggcgcgggcctgtacgtgggctgggcggccgcggcgctgcagctgctggggggcgcgctgctctgctgctcgtgtcccccacgcgagaagaagtacacggccaccaaggtcgtctactccgcgccgcgctccaccggcccgggagccagcctgggcacaggctacgaccgcaaggactacgtcacgcgtac

SEQ ID NO: 198 - protein, 214 aa, synthetic construct
  REGION 1..214: note = Synthetic peptide
DIVLTQSPATLSVTPGNSVSLSCRASQSIGGNLHWYQQKSHESPRLLIKYASQSISGIPSRFSGSGSGTDFTLSINSVETEDFGMYFCQQSNSWPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 199 - protein, 444 aa, synthetic construct
  REGION 1..444: note = Synthetic peptide
DVQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 200 - protein, 220 aa, synthetic construct; identical to SEQ ID NO: 190
  REGION 1..220: note = Synthetic peptide
  VARIANT 143: note = Xaa can be any naturally occurring amino acid

SEQ ID NO: 201 - protein, 220 aa, synthetic construct; identical to SEQ ID NO: 190
  REGION 1..220: note = Synthetic peptide
  VARIANT 143: note = Xaa can be any naturally occurring amino acid

SEQ ID NO: 202 - protein, 220 aa, Homo sapiens; identical to SEQ ID NO: 190
  REGION 1..220: note = human CLDN6
  VARIANT 143: note = X is I or V

SEQ ID NO: 203-207 - skipped (5 entries)

SEQ ID NO: 208 - DNA, 21 bp, synthetic construct
  misc_feature 1..21: note = Synthetic polynucleotide
  source 1..21: mol_type = other DNA
tccagtgtaagttccacttac

SEQ ID NO: 209 - skipped

SEQ ID NO: 210 - DNA, 27 bp, synthetic construct
  misc_feature 1..27: note = Synthetic polynucleotide
  source 1..27: mol_type = other DNA
caccagtatcatcgttccccgctcacg

SEQ ID NO: 211 - DNA, 24 bp, synthetic construct
  misc_feature 1..24: note = Synthetic polynucleotide
  source 1..24: mol_type = other DNA
ggctacacctttactacctacacg

SEQ ID NO: 212 - DNA, 24 bp, synthetic construct
  misc_feature 1..24: note = Synthetic polynucleotide
  source 1..24: mol_type = other DNA
attaatcctagcagtggatatact

SEQ ID NO: 213 - DNA, 27 bp, synthetic construct
  misc_feature 1..27: note = Synthetic polynucleotide
  source 1..27: mol_type = other DNA
gcaaacggggattactacgtcgcttac

SEQ ID NO: 214 - DNA, 18 bp, synthetic construct
  misc_feature 1..18: note = Synthetic polynucleotide
  source 1..18: mol_type = other DNA
gagaatatttacagttat

SEQ ID NO: 215 - skipped

SEQ ID NO: 216 - DNA, 27 bp, synthetic construct
  misc_feature 1..27: note = Synthetic polynucleotide
  source 1..27: mol_type = other DNA
caacatcattatactgttccgtggacg

SEQ ID NO: 217 - DNA, 24 bp, synthetic construct
  misc_feature 1..24: note = Synthetic polynucleotide
  source 1..24: mol_type = other DNA
ggtttcactttcagtgattactgg

SEQ ID NO: 218 - DNA, 30 bp, synthetic construct
  misc_feature 1..30: note = Synthetic polynucleotide
  source 1..30: mol_type = other DNA
attagattgaaatctgataattatgcaaca

SEQ ID NO: 219 - DNA, 24 bp, synthetic construct
  misc_feature 1..24: note = Synthetic polynucleotide
  source 1..24: mol_type = other DNA
aatgatggccccccctcggggtgt

SEQ ID NO: 220 - DNA, 18 bp, synthetic construct
  misc_feature 1..18: note = Synthetic polynucleotide
  source 1..18: mol_type = other DNA
gagaatatttacagttat

SEQ ID NO: 221 - skipped

SEQ ID NO: 222 - DNA, 27 bp, synthetic construct
  misc_feature 1..27: note = Synthetic polynucleotide
  source 1..27: mol_type = other DNA
caacatcattatactgttccgtggacg

SEQ ID NO: 223 - DNA, 24 bp, synthetic construct
  misc_feature 1..24: note = Synthetic polynucleotide
  source 1..24: mol_type = other DNA
ggattcactttcagtaattactgg

SEQ ID NO: 224 - DNA, 30 bp, synthetic construct; identical to SEQ ID NO: 218
  misc_feature 1..30: note = Synthetic polynucleotide
  source 1..30: mol_type = other DNA

SEQ ID NO: 225 - DNA, 24 bp, synthetic construct
  misc_feature 1..24: note = Synthetic polynucleotide
  source 1..24: mol_type = other DNA
aatgatggccccccctcggggtgt

SEQ ID NO: 226 - DNA, 33 bp, synthetic construct
  misc_feature 1..33: note = Synthetic polynucleotide
  source 1..33: mol_type = other DNA
cagagccttgtacacagtgatggaaacacctat

SEQ ID NO: 227 - skipped

SEQ ID NO: 228 - DNA, 27 bp, synthetic construct
  misc_feature 1..27: note = Synthetic polynucleotide
  source 1..27: mol_type = other DNA
tctcaaagtacacatgttccttacacg

SEQ ID NO: 229 - DNA, 24 bp, synthetic construct
  misc_feature 1..24: note = Synthetic polynucleotide
  source 1..24: mol_type = other DNA
ggctacacctttactagctacacg

SEQ ID NO: 230 - DNA, 24 bp, synthetic construct
  misc_feature 1..24: note = Synthetic polynucleotide
  source 1..24: mol_type = other DNA
attaatcctagcagtacttatact

SEQ ID NO: 231 - DNA, 30 bp, synthetic construct
  misc_feature 1..30: note = Synthetic polynucleotide
  source 1..30: mol_type = other DNA
tcaagaggggaactgggagggtttgcttac

SEQ ID NO: 232 - DNA, 33 bp, synthetic construct
  misc_feature 1..33: note = Synthetic polynucleotide
  source 1..33: mol_type = other DNA
cagagcattgtacatagtaatggaaacacctat

SEQ ID NO: 233 - skipped

SEQ ID NO: 234 - DNA, 27 bp, synthetic construct
  misc_feature 1..27: note = Synthetic polynucleotide
  source 1..27: mol_type = other DNA
tttcaaggttcacatgttccattcacg

SEQ ID NO: 235 - DNA, 24 bp, synthetic construct
  misc_feature 1..24: note = Synthetic polynucleotide
  source 1..24: mol_type = other DNA
ggatacatattcactcactatatt

SEQ ID NO: 236 - DNA, 24 bp, synthetic construct
  misc_feature 1..24: note = Synthetic polynucleotide
  source 1..24: mol_type = other DNA
attaatccttacaatgatggtact

SEQ ID NO: 237 - DNA, 39 bp, synthetic construct
  misc_feature 1..39: note = Synthetic polynucleotide
  source 1..39: mol_type = other DNA
gcaagatactacggctacccttactattctatggactac

SEQ ID NO: 238 - DNA, 36 bp, synthetic construct
  misc_feature 1..36: note = Synthetic polynucleotide
  source 1..36: mol_type = other DNA
cagagtctgctcaacagtagaacccgaaagaactac

SEQ ID NO: 239 - skipped

SEQ ID NO: 240 - DNA, 24 bp, synthetic construct
  misc_feature 1..24: note = Synthetic polynucleotide
  source 1..24: mol_type = other DNA
aagcaatcttattatctgtacacg

SEQ ID NO: 241 - DNA, 27 bp, synthetic construct
  misc_feature 1..27: note = Synthetic polynucleotide
  source 1..27: mol_type = other DNA
ggctactccatcaccagtggttattac

SEQ ID NO: 242 - DNA, 21 bp, synthetic construct
  misc_feature 1..21: note = Synthetic polynucleotide
  source 1..21: mol_type = other DNA
atcagctacgatggtggcatt

SEQ ID NO: 243 - DNA, 30 bp, synthetic construct
  misc_feature 1..30: note = Synthetic polynucleotide
  source 1..30: mol_type = other DNA
gcaagatttggtaagggggctatggactac

SEQ ID NO: 244 - DNA, 21 bp, synthetic construct
  misc_feature 1..21: note = Synthetic polynucleotide
  source 1..21: mol_type = other DNA
tcaagtgtaagttccagttac

SEQ ID NO: 245 - skipped

SEQ ID NO: 246 - DNA, 27 bp, synthetic construct
  misc_feature 1..27: note = Synthetic polynucleotide
  source 1..27: mol_type = other DNA
caccagtatcatcgttccccacccacg

SEQ ID NO: 247 - DNA, 24 bp, synthetic construct
  misc_feature 1..24: note = Synthetic polynucleotide
  source 1..24: mol_type = other DNA
ggttactcattcactggctacacc

SEQ ID NO: 248 - DNA, 24 bp, synthetic construct
  misc_feature 1..24: note = Synthetic polynucleotide
  source 1..24: mol_type = other DNA
attaatccttacaatggtggtact

SEQ ID NO: 249 - DNA, 36 bp, synthetic construct
  misc_feature 1..36: note = Synthetic polynucleotide
  source 1..36: mol_type = other DNA
gcaagaggggtctatgattacgacggatttacttac

SEQ ID NO: 250 - DNA, 33 bp, synthetic construct; identical to SEQ ID NO: 226
  misc_feature 1..33: note = Synthetic polynucleotide
  source 1..33: mol_type = other DNA

SEQ ID NO: 251 - skipped

SEQ ID NO: 252 - DNA, 27 bp, synthetic construct
  misc_feature 1..27: note = Synthetic polynucleotide
  source 1..27: mol_type = other DNA
tctcaaagtacacatgttccttacacg

SEQ ID NO: 253 - DNA, 24 bp, synthetic construct
  misc_feature 1..24: note = Synthetic polynucleotide
  source 1..24: mol_type = other DNA
ggctacacctttactacctacacg

SEQ ID NO: 254 - DNA, 24 bp, synthetic construct
  misc_feature 1..24: note = Synthetic polynucleotide
  source 1..24: mol_type = other DNA
attaatcctcgcagtggttatagt

SEQ ID NO: 255 - DNA, 30 bp, synthetic construct; identical to SEQ ID NO: 231
  misc_feature 1..30: note = Synthetic polynucleotide
  source 1..30: mol_type = other DNA

SEQ ID NO: 256 - DNA, 18 bp, synthetic construct
  misc_feature 1..18: note = Synthetic polynucleotide
  source 1..18: mol_type = other DNA
cagaccattggtacatgg

SEQ ID NO: 257 - skipped

SEQ ID NO: 258 - DNA, 27 bp, synthetic construct
  misc_feature 1..27: note = Synthetic polynucleotide
  source 1..27: mol_type = other DNA
caacaactttacagtattcctcggacg

SEQ ID NO: 259 - DNA, 24 bp, synthetic construct
  misc_feature 1..24: note = Synthetic polynucleotide
  source 1..24: mol_type = other DNA
ggatacagattcactgactacaac

SEQ ID NO: 260 - DNA, 24 bp, synthetic construct
  misc_feature 1..24: note = Synthetic polynucleotide
  source 1..24: mol_type = other DNA
attaaccctaacaatggtggtact

SEQ ID NO: 261 - DNA, 30 bp, synthetic construct
  misc_feature 1..30: note = Synthetic polynucleotide
  source 1..30: mol_type = other DNA
gcaagagattacttgtacttctttgactgc

SEQ ID NO: 262 - DNA, 33 bp, synthetic construct
  misc_feature 1..33: note = Synthetic polynucleotide
  source 1..33: mol_type = other DNA
cagagccttgtacacagtaatggaaacacctat

SEQ ID NO: 263 - skipped

SEQ ID NO: 264 - DNA, 27 bp, synthetic construct
  misc_feature 1..27: note = Synthetic polynucleotide
  source 1..27: mol_type = other DNA
tctcaaattacacatgttccgtacacg

SEQ ID NO: 265 - DNA, 24 bp, synthetic construct
  misc_feature 1..24: note = Synthetic polynucleotide
  source 1..24: mol_type = other DNA
ggttataccttcacagactattca

SEQ ID NO: 266 - DNA, 24 bp, synthetic construct
  misc_feature 1..24: note = Synthetic polynucleotide
  source 1..24: mol_type = other DNA
ataagcactgagactggtgagcca

SEQ ID NO: 267 - DNA, 30 bp, synthetic construct
  misc_feature 1..30: note = Synthetic polynucleotide
  source 1..30: mol_type = other DNA
actagaggtctatggtcctcgtttgcttac

SEQ ID NO: 268 - DNA, 30 bp, synthetic construct
  misc_feature 1..30: note = Synthetic polynucleotide
  source 1..30: mol_type = other DNA
aaaagtgtcagtacatctggctatagttat

SEQ ID NO: 269 - skipped

SEQ ID NO: 270 - DNA, 27 bp, synthetic construct
  misc_feature 1..27: note = Synthetic polynucleotide
  source 1..27: mol_type = other DNA
cagcacagtagggagcttccgctcacg

SEQ ID NO: 271 - DNA, 24 bp, synthetic construct
  misc_feature 1..24: note = Synthetic polynucleotide
  source 1..24: mol_type = other DNA
ggattcactttcagtagctttgga

SEQ ID NO: 272 - DNA, 24 bp, synthetic construct
  misc_feature 1..24: note = Synthetic polynucleotide
  source 1..24: mol_type = other DNA
attagtagtgacagtaggaccatc

SEQ ID NO: 273 - DNA, 33 bp, synthetic construct
  misc_feature 1..33: note = Synthetic polynucleotide
  source 1..33: mol_type = other DNA
gcaagagactacggtagaacctacgaggcttac

SEQ ID NO: 274 - DNA, 18 bp, synthetic construct
  misc_feature 1..18: note = Synthetic polynucleotide
  source 1..18: mol_type = other DNA
caggacattggaggaaat

SEQ ID NO: 275 - skipped

SEQ ID NO: 276 - DNA, 27 bp, synthetic construct
  misc_feature 1..27: note = Synthetic polynucleotide
  source 1..27: mol_type = other DNA
ctacagcgtaatgcgtatccgctcact

SEQ ID NO: 277 - DNA, 24 bp, synthetic construct
  misc_feature 1..24: note = Synthetic polynucleotide
  source 1..24: mol_type = other DNA
ggattcactttcagtagttatgcc

SEQ ID NO: 278 - DNA, 21 bp, synthetic construct
  misc_feature 1..21: note = Synthetic polynucleotide
  source 1..21: mol_type = other DNA
attagaagtggtggtaccacc

SEQ ID NO: 279 - DNA, 36 bp, synthetic construct
  misc_feature 1..36: note = Synthetic polynucleotide
  source 1..36: mol_type = other DNA
gcaaaagtgggcggtaacccctatcctatggactac

SEQ ID NO: 280 - DNA, 21 bp, synthetic construct
  misc_feature 1..21: note = Synthetic polynucleotide
  source 1..21: mol_type = other DNA
tcaagtataagttccaattac

SEQ ID NO: 281 - skipped

SEQ ID NO: 282 - DNA, 27 bp, synthetic construct
  misc_feature 1..27: note = Synthetic polynucleotide
  source 1..27: mol_type = other DNA
cagcagggtagtagtataccgctcacg

SEQ ID NO: 283 - DNA, 24 bp, synthetic construct
  misc_feature 1..24: note = Synthetic polynucleotide
  source 1..24: mol_type = other DNA
ggatacgccttcagtaattacttg

SEQ ID NO: 284 - DNA, 24 bp, synthetic construct
  misc_feature 1..24: note = Synthetic polynucleotide
  source 1..24: mol_type = other DNA
attaatcctggaagtggtggtact

SEQ ID NO: 285 - DNA, 45 bp, synthetic construct
  misc_feature 1..45: note = Synthetic polynucleotide
  source 1..45: mol_type = other DNA
gcaagatcatacttcggtagaagctacccctatactatggactac

SEQ ID NO: 286 - DNA, 30 bp, synthetic construct
  misc_feature 1..30: note = Synthetic polynucleotide
  source 1..30: mol_type = other DNA
caaagtgttgattatgatggtgataattat

SEQ ID NO: 287 - skipped

SEQ ID NO: 288 - DNA, 27 bp, synthetic construct
  misc_feature 1..27: note = Synthetic polynucleotide
  source 1..27: mol_type = other DNA
cagcaaagtaatgaggatccattcacg

SEQ ID NO: 289 - DNA, 24 bp, synthetic construct
  misc_feature 1..24: note = Synthetic polynucleotide
  source 1..24: mol_type = other DNA
ggctacacattcactgattatgct

SEQ ID NO: 290 - DNA, 24 bp, synthetic construct
  misc_feature 1..24: note = Synthetic polynucleotide
  source 1..24: mol_type = other DNA
attagtacatactctggtaataca

SEQ ID NO: 291 - DNA, 39 bp, synthetic construct
  misc_feature 1..39: note = Synthetic polynucleotide
  source 1..39: mol_type = other DNA
gcaagaaggggcgattacagcctctatgctatggactac

SEQ ID NO: 292 - DNA, 36 bp, synthetic construct
  misc_feature 1..36: note = Synthetic polynucleotide
  source 1..36: mol_type = other DNA
caaagtgttttattcagttcaaatcagaaaaactac

SEQ ID NO: 293 - skipped

SEQ ID NO: 294 - DNA, 24 bp, synthetic construct
  misc_feature 1..24: note = Synthetic polynucleotide
  source 1..24: mol_type = other DNA
catcaatacctctcctcgcgcacg

SEQ ID NO: 295 - DNA, 24 bp, synthetic construct
  misc_feature 1..24: note = Synthetic polynucleotide
  source 1..24: mol_type = other DNA
ggattcactttcagtagctttgga

SEQ ID NO: 296 - DNA, 24 bp, synthetic construct
  misc_feature 1..24: note = Synthetic polynucleotide
  source 1..24: mol_type = other DNA
attagtagtgacagtaggaccatc

SEQ ID NO: 297 - DNA, 33 bp, synthetic construct; identical to SEQ ID NO: 273
  misc_feature 1..33: note = Synthetic polynucleotide
  source 1..33: mol_type = other DNA

SEQ ID NO: 298 - DNA, 30 bp, synthetic construct
  misc_feature 1..30: note = Synthetic polynucleotide
  source 1..30: mol_type = other DNA
gaaagtgttgataattatggcattagtttt

SEQ ID NO: 299 - skipped

SEQ ID NO: 300 - DNA, 27 bp, synthetic construct
  misc_feature 1..27: note = Synthetic polynucleotide
  source 1..27: mol_type = other DNA
cagcaaagtaaggaggttccgctcacg

SEQ ID NO: 301 - DNA, 24 bp, synthetic construct
  misc_feature 1..24: note = Synthetic polynucleotide
  source 1..24: mol_type = other DNA
ggattccctttcagtagctctgcc

SEQ ID NO: 302 - DNA, 21 bp, synthetic construct
  misc_feature 1..21: note = Synthetic polynucleotide
  source 1..21: mol_type = other DNA
attaatagtgatggtaacacc

SEQ ID NO: 303 - DNA, 39 bp, synthetic construct
  misc_feature 1..39: note = Synthetic polynucleotide
  source 1..39: mol_type = other DNA
acaagaaacggggactataggtacgacgagtttgcttac

SEQ ID NO: 304 - DNA, 21 bp, synthetic construct
  misc_feature 1..21: note = Synthetic polynucleotide
  source 1..21: mol_type = other DNA
tcaagtgtaagttccagttac

SEQ ID NO: 305 - skipped

SEQ ID NO: 306 - DNA, 27 bp, synthetic construct
  misc_feature 1..27: note = Synthetic polynucleotide
  source 1..27: mol_type = other DNA
caccagtatcatcgttccccacccacg

SEQ ID NO: 307 - DNA, 24 bp, synthetic construct
  misc_feature 1..24: note = Synthetic polynucleotide
  source 1..24: mol_type = other DNA
ggctacacctttactggctactgg

SEQ ID NO: 308 - DNA, 24 bp, synthetic construct
  misc_feature 1..24: note = Synthetic polynucleotide
  source 1..24: mol_type = other DNA
attaatcctagcactggttatact

SEQ ID NO: 309 - DNA, 36 bp, synthetic construct
  misc_feature 1..36: note = Synthetic polynucleotide
  source 1..36: mol_type = other DNA
gcaagagaggggattactactgtgctggttgactac

SEQ ID NO: 310 - DNA, 36 bp, synthetic construct; identical to SEQ ID NO: 292
  misc_feature 1..36: note = Synthetic polynucleotide
  source 1..36: mol_type = other DNA

SEQ ID NO: 311 - skipped

SEQ ID NO: 312 - DNA, 24 bp, synthetic construct
  misc_feature 1..24: note = Synthetic polynucleotide
  source 1..24: mol_type = other DNA
catcaatacctctcctcgcgcacg

SEQ ID NO: 313 - DNA, 24 bp, synthetic construct
  misc_feature 1..24: note = Synthetic polynucleotide
  source 1..24: mol_type = other DNA
ggttactctttcactggctacaat

SEQ ID NO: 314 - DNA, 24 bp, synthetic construct
  misc_feature 1..24: note = Synthetic polynucleotide
  source 1..24: mol_type = other DNA
attgatccttactatggtggttct

SEQ ID NO: 315 - DNA, 42 bp, synthetic construct
  misc_feature 1..42: note = Synthetic polynucleotide
  source 1..42: mol_type = other DNA
gcaagagagaggtcgggctacgttttctctgctatggactac

SEQ ID NO: 316 - DNA, 36 bp, synthetic construct; identical to SEQ ID NO: 292
  misc_feature 1..36: note = Synthetic polynucleotide
  source 1..36: mol_type = other DNA

SEQ ID NO: 317 - skipped

SEQ ID NO: 318 - DNA, 24 bp, synthetic construct
  misc_feature 1..24: note = Synthetic polynucleotide
  source 1..24: mol_type = other DNA
catcaatacctctcctcgcgcacg

SEQ ID NO: 319 - DNA, 24 bp, synthetic construct
  misc_feature 1..24: note = Synthetic polynucleotide
  source 1..24: mol_type = other DNA
ggttactcattcactggctacacc

SEQ ID NO: 320 - DNA, 24 bp, synthetic construct
  misc_feature 1..24: note = Synthetic polynucleotide
  source 1..24: mol_type = other DNA
attaatccttacaatggtgttact

SEQ ID NO: 321 - DNA, 48 bp, synthetic construct
  misc_feature 1..48: note = Synthetic polynucleotide
  source 1..48: mol_type = other DNA
acaagagatcccctttactacggctacagggactctactatggactac

SEQ ID NO: 322 - DNA, 33 bp, synthetic construct; identical to SEQ ID NO: 226
  misc_feature 1..33: note = Synthetic polynucleotide
  source 1..33: mol_type = other DNA

SEQ ID NO: 323 - skipped

SEQ ID NO: 324 - DNA, 27 bp, synthetic construct
  misc_feature 1..27: note = Synthetic polynucleotide
  source 1..27: mol_type = other DNA
tctcaaagtacacatgttccttacacg

SEQ ID NO: 325 - DNA, 24 bp, synthetic construct
  misc_feature 1..24: note = Synthetic polynucleotide
  source 1..24: mol_type = other DNA
ggctacacctttactagctacacg

SEQ ID NO: 326 - DNA, 24 bp, synthetic construct
  misc_feature 1..24: note = Synthetic polynucleotide
  source 1..24: mol_type = other DNA
attaatcctagcagtacgtatact

SEQ ID NO: 327 - DNA, 30 bp, synthetic construct; identical to SEQ ID NO: 231
  misc_feature 1..30: note = Synthetic polynucleotide
  source 1..30: mol_type = other DNA

SEQ ID NO: 328 - DNA, 18 bp, synthetic construct
  misc_feature 1..18: note = Synthetic polynucleotide
  source 1..18: mol_type = other DNA
cagggcattagaggtaat

SEQ ID NO: 329 - skipped

SEQ ID NO: 330 - DNA, 27 bp, synthetic construct
  misc_feature 1..27: note = Synthetic polynucleotide
  source 1..27: mol_type = other DNA
ctacagcgtaatgcgtatcctctcacg

SEQ ID NO: 331 - DNA, 24 bp, synthetic construct
  misc_feature 1..24: note = Synthetic polynucleotide
  source 1..24: mol_type = other DNA
ggattcactttcagtagttttgcc

SEQ ID NO: 332 - DNA, 21 bp, synthetic construct
  misc_feature 1..21: note = Synthetic polynucleotide
  source 1..21: mol_type = other DNA
attagaagtggtggtattacc

SEQ ID NO: 333 - DNA, 39 bp, synthetic construct
  misc_feature 1..39: note = Synthetic polynucleotide
  source 1..39: mol_type = other DNA
gcaagagttagtacggctacgtactatggtatggactac

SEQ ID NO: 334 - DNA, 325 bp, synthetic construct
  misc_feature 1..325: note = Synthetic polynucleotide
  source 1..325: mol_type = other DNA
caaattgttctcacccagtctccagcaatcatgtctgcatctctaggggaacgggtcaccatgacctgcactgccagctccagtgtaagttccacttactttcactggtaccaacagaagccaggatcctcccccaaactctggatttatagcacatccaacctggcttctggagtcccacgtcgcttcagtggcagtgcgtctgggacctcttactctctcacaatcagcagcatggaggctgaagatgctgccacttattattgccaccagtatcatcgttccccgctcacgttcggtgctgggaccaagctggagctgaaac

SEQ ID NO: 335 - DNA, 349 bp, synthetic construct
  misc_feature 1..349: note = Synthetic polynucleotide
  source 1..349: mol_type = other DNA
caggtccagctgcagcagtctgcagctgaactggcaagacctggggcctcagtgaagatgtcctgcaaggcttctggctacacctttactacctacacgatgcactgggtaaaacagaggcctggacagggtctggaatggattggattcattaatcctagcagtggatatactgactacaatcagaagttcaaggacaggaccacattgactgcagacaaatcctccagcacagtctacatgcaactgagtagcctgacatctgaggactctgcggtctattactgtgcaaacggggattactacgtcgcttactggggccaagggactctggtcactgtctctgcag

SEQ ID NO: 336 - DNA, 322 bp, synthetic construct
  misc_feature 1..322: note = Synthetic polynucleotide
  source 1..322: mol_type = other DNA
gacatccagatgactcagtctccagcctccctatctgcatctgtgggagaaactgtcaccatcacatgtcgaataagcgagaatatttacagttatttagcatggtatcagcagaaacagggaaaatctcctcagctcctggtctataatgcaaaaatcttagtagaaggtgtgccatcaaggttcagtggcagtggatcaggcacacagttttctctgaagatcaacagcctgcagcctgaagattttgggaattattactgtcaacatcattatactgttccgtggacgttcggtggaggcaccaaactggaaatcaaac

SEQ ID NO: 337 - DNA, 352 bp, synthetic construct
  misc_feature 1..352: note = Synthetic polynucleotide
  source 1..352: mol_type = other DNA
gaagtgaagcttgaggagtctggaggaggcttggtgcaacctggaggatccatgaaactctcctgtgttgcctctggtttcactttcagtgattactggatgaactgggtccgccagtctccagagaaggggcttgaatgggttgctcaaattagattgaaatctgataattatgcaacacattatgcggagtctgtgaaagggaggttcaccatctcaagagatgattccaaaagaagtgtctacctgcaaatgaacaacttaagggctgaagacactggaacttattactgcaatgatggccccccctcggggtgttggggccaaggcaccactctcatagtctcctcag

SEQ ID NO: 338 - DNA, 322 bp, synthetic construct
  misc_feature 1..322: note = Synthetic polynucleotide
  source 1..322: mol_type = other DNA
gacatccagatgactcagtctccagcctccctatctgcatctgtgggagaaactgtcaccatcacatgtcgaataagtgagaatatttacagttatttagcatggtatcagcagaaacagggaaaatctcctcagctcctggtctataatgcaaaaatcttagtagaaggtgtgccatcaaggttcagtggcagtggatcaggcacacagttttctctgaagatcaacagcctgcagcctgaagattttgggaattattactgtcaacatcattatactgttccgtggacgttcggtggaggcaccaaactggaaatcaaac

SEQ ID NO: 339 - DNA, 352 bp, synthetic construct
  misc_feature 1..352: note = Synthetic polynucleotide
  source 1..352: mol_type = other DNA
gaagtgaagcttgaggagtctggaggaggcttggtgcaacctggaggatccatgaaactctcctgtgttgcctctggattcactttcagtaattactggatgaactgggtccgccagtctccagagaaggggcttgaatgggttgctcaaattagattgaaatctgataattatgcaacacattatgcggagtctgtgaaagggaggttcaccatctcaagagatgattccaaaagaagtgtctacctgcaaatgaacaacttaagggctgaagacactggaacttattactgcaatgatggccccccctcggggtgttggggccaaggcaccactctcatagtctcctcag

SEQ ID NO: 340 - DNA, 337 bp, synthetic construct
  misc_feature 1..337: note = Synthetic polynucleotide
  source 1..337: mol_type = other DNA
gatgttgtgatgacccaaactccactctccctgcctgtcagtcttggagatcaagcctccatctcttgcagatctagtcagagccttgtacacagtgatggaaacacctatttaaattggtacctgcagaagccaggccagtctccaaagctcctgatctacaaagtttccaaccgtttttctggggtcccagacaggttcagtggcagtggatcagggacagatttcacactcaagatcaggagagtggaggctgaggatctgggagtttatttctgctctcaaagtacacatgttccttacacgttcggaggggggaccaagctggaaataaaac

SEQ ID NO: 341 - DNA, 352 bp, synthetic construct
  misc_feature 1..352: note = Synthetic polynucleotide
  source 1..352: mol_type = other DNA
caggtccagttgcagcagtctggggctgaactggcaagacctggggcctcagtgaagatgtcctgcaaggcttctggctacacctttactagctacacgatgcactggataaaacagagacctggacagggtcaggaatggattggatacattaatcctagcagtacttatactcattacattaagaaattcaaggacaaggccacattgactgcagacaaatcctccagcacagcctacatgcaactgcgcagcctgacatctgaggactctgcagtctattactgttcaagaggggaactgggagggtttgcttactggggccaagggactctggtcactgtctctgcag

SEQ ID NO: 342 - DNA, 337 bp, synthetic construct
  misc_feature 1..337: note = Synthetic polynucleotide
  source 1..337: mol_type = other DNA
gatgttttgatgacccaaactccactctccctgcctgtcagtcttggagatcaaccctccatctcttgcagatctagtcagagcattgtacatagtaatggaaacacctatttagattggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaggttggaaataaaac

SEQ ID NO: 343 - DNA, 361 bp, synthetic construct
  misc_feature 1..361: note = Synthetic polynucleotide
  source 1..361: mol_type = other DNA
gaagtccagctgcagcagtctggacctgagctggtaaagcctggggcttcagtgaagatgtcctgcaaggcttctggatacatattcactcactatattatgcactgggtgaagcagaagcctgggcagggccttgagtggattggatgtattaatccttacaatgatggtactaagtacaatgagaagttcaaaggcaaggccacactgacttcagacaaatcctccagcacagcctacatggagctcagcagcctgacctctgaggactctgcggtctattactgtgcaagatactacggctacccttactattctatggactactggggtcaaggaacctcagtcaccgtctcctcag

SEQ ID NO: 344 - DNA, 337 bp, synthetic construct
  misc_feature 1..337: note = Synthetic polynucleotide
  source 1..337: mol_type = other DNA
gccattgtgatgttccagtctccatcctccctggttgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttattatctgtacacgttcggaggggggaccaagctggaaataaaac

SEQ ID NO: 345 - DNA, 352 bp, synthetic construct
  misc_feature 1..352: note = Synthetic polynucleotide
  source 1..352: mol_type = other DNA
gatgtgcagcttcaggagtcaggacctggcctcgtgaaatcttctcagtctctgtctctcacctgctctgtcactggctactccatcaccagtggttattactggaaatggatccggcagtttccaggaaacaaactggaatggatgggctacatcagctacgatggtggcattaactacaacccatctctcaaaaatcgaatctccatcactcgtgacacatctaagaaccagtttttcctgaagttgaattctgtgactactgaggacacagccaaatattactgtgcaagatttggtaagggggctatggactactggggtcaaggaacctcagtcaccgtctcctcag

SEQ ID NO: 346 - DNA, 325 bp, synthetic construct
  misc_feature 1..325: note = Synthetic polynucleotide
  source 1..325: mol_type = other DNA
caaattgttctcacccagtctccagcaatcatgtctgcatctctgggggaccgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccacgttcggctcggggacaaagttggaaataaaac

SEQ ID NO: 347 - DNA, 358 bp, synthetic construct
  misc_feature 1..358: note = Synthetic polynucleotide
  source 1..358: mol_type = other DNA
gaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtactaactacaaccagaagttcaagggcaaggccacattaactgtagacaagtcatccagcacagcctacatggagctcctcagtctgacatctgaggactctgcagtctattactgtgcaagaggggtctatgattacgacggatttacttactggggccaagggactctggtcactgtctctgcag

SEQ ID NO: 348 - DNA, 337 bp, synthetic construct
  misc_feature 1..337: note = Synthetic polynucleotide
  source 1..337: mol_type = other DNA
gatgttgtgatgacccaaactccactctccctgcctgtcagtcttggagatcaagcctccatctcttgcagatctagtcagagccttgtacacagtgatggaaacacctatttatattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccttacacgttcggaggggggaccaagctggaaataaaac

SEQ ID NO: 349 - DNA, 352 bp, synthetic construct
  misc_feature 1..352: note = Synthetic polynucleotide
  source 1..352: mol_type = other DNA
caggtccagctgcagcagtctggggctgaactggcaagacctggggcctcagtgaagatgtcctgcaaggcttctggctacacctttactacctacacgatgcactggttaaaacagaggcctggacagggtctggaatggattggatacattaatcctcgcagtggttatagtaattacaatcagaagttcaaggacaaggccacattgactgcagacaagtcctccaacacagcctacatgcaactgaacaccctgacatctgaggactctaaagtctattactgttcaagaggggaactgggagggtttgcttactggggccaagggactctggtcactgtctctgcag

SEQ ID NO: 350 - DNA, 322 bp, synthetic construct
  misc_feature 1..322: note = Synthetic polynucleotide
  source 1..322: mol_type = other DNA
gacattcagatgacccagtctcctgcctcccagtctgcatctctgggagaaagtgtcaccatcacatgcctggcaagtcagaccattggtacatggttagcatggtatcagcagaaaccagggaaatctcctcagctcctgatttatgctgcagccagcttggcagatggggtcccatcaaggttcagtggtagtggatctggcacaagattttctttcaagatcagcagcctacaggctgaagattttgtaagttattactgtcaacaactttacagtattcctcggacgttcggtggaggcaccaagctggaaatcaaac

SEQ ID NO: 351 - DNA, 352 bp, synthetic construct
  misc_feature 1..352: note = Synthetic polynucleotide
  source 1..352: mol_type = other DNA
gaggtccagctgcaacagtctggacctgagctggtgaagcctggggcttcagtgaagatgtcctgcaaggcttctggatacagattcactgactacaacatgcactgggtgaagcagagccatggaaagagccttgagtggattggatatattaaccctaacaatggtggtactaactacaaccaaaacttcaagggcaaggccacattgactgtgaacaagtcctccagcacagcctacatggagctccgcagcctgacatcggaggattctgcagcctattactgtgcaagagattacttgtacttctttgactgctggggccaaggcaccactctcacagtctcctcag

SEQ ID NO: 352 - DNA, 337 bp, synthetic construct
  misc_feature 1..337: note = Synthetic polynucleotide
  source 1..337: mol_type = other DNA
gatgttgtgatgacccaaactccactctccctgcctgtcagtcttggagatcaagcctccatctcttgcagagctagtcagagccttgtacacagtaatggaaacacctatttacattggttcctgcagaagccaggccagtctccaaagctcctgatctacaaagtttccaaccgattttctggggtcccagacaggttcagtggcagtggatcacggacagatttcacactcaagatcagcagagtggaggctgaggatctgggagtttatttctgctctcaaattacacatgttccgtacacgttcggaggggggaccaagctggaaataaaac

SEQ ID NO: 353 - DNA, 352 bp, synthetic construct
  misc_feature 1..352: note = Synthetic polynucleotide
  source 1..352: mol_type = other DNA
caaatccagttggtgcagtctggacctgcgctgaagaagcctggagagacagtcaagatctcctgcaaggcttctggttataccttcacagactattcaatgcactggataaagcaggctccaggaaagggtttaaagtggatgggctggataagcactgagactggtgagccaacatatgcagatggcttcaagggacggtttgacttctctttggaaacctctgccgacactgcctatttgtccatcaacaacctcacaaatgaggacacggctacatatttctgtactagaggtctatggtcctcgtttgcttactggggccaagggactctggtcactgtctctgcag

SEQ ID NO: 354 - DNA, 334 bp, synthetic construct
  misc_feature 1..334: note = Synthetic polynucleotide
  source 1..334: mol_type = other DNA
gacattgtgctgacacagtctcctgcttccttagctgtatctctggggcagagggccaccatctcatgcagggccagcaaaagtgtcagtacatctggctatagttatatacactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgctcacgttcggtgctgggaccaagctggagctgaaac

SEQ ID NO: 355 - DNA, 355 bp, synthetic construct
  misc_feature 1..355: note = Synthetic polynucleotide
  source 1..355: mol_type = other DNA
gatgtgcagctggtggagtctgggggaggcttggtgcagcctagagggtcccggaaactctcctgtgcagcctctggattcactttcagtagctttggaatgcactgggttcgtcaggctccagagaaggggctggagtgggtcgcatacattagtagtgacagtaggaccatctattatgcagacacagtgaagggccgattcaccatctccagagacaatcccacgaacaccctgttcctgcaaatgaccagtctcaggtctgaggacacggccatgtattactgtgcaagagactacggtagaacctacgaggcttactggggccaagggactctggtcactgtctctgcag

SEQ ID NO: 356 - DNA, 322 bp, synthetic construct
  misc_feature 1..322: note = Synthetic polynucleotide
  source 1..322: mol_type = other DNA
gacatccagatgattcagtctccatcgtccatgtttgcctctctgggagacagagtcagtctctcttgtcgggctagtcaggacattggaggaaatttagactggtatcagcagaaaccaggtggaactattaaactcctgatctactccacatccaatttaaattctggtgtcccatcaaggttcagtggcagtgggtctgggtcagattattctctcaccatcaccagcctggagtctgaagattttgcagactattactgtctacagcgtaatgcgtatccgctcactttcggtgctgggaccaagctggagctgaaac

SEQ ID NO: 357 - DNA, 355 bp, synthetic construct
  misc_feature 1..355: note = Synthetic polynucleotide
  source 1..355: mol_type = other DNA
gaagtgaaactgatggagtctgggggagacttagtgaagcctggagggtccctgaaactctcctgtgcagcctctggattcactttcagtagttatgccatgtcttgggttcgccagactccagagaagaggctggagtgggtcgcgtccattagaagtggtggtaccacctactatccagacagtgtgaagggccgattcaccatctccagagataatgccaggaacatcctgtacctgcgaatgagtagtctgaggtctgaggacacggccatatattactgtgcaaaagtgggcggtaacccctatcctatggactactggggtcaaggaacctcagtcaccgtctcctcag

SEQ ID NO: 358 - DNA, 325 bp, synthetic construct
  misc_feature 1..325: note = Synthetic polynucleotide
  source 1..325: mol_type = other DNA
gaaattgtgctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccgctcacgttcggtgctgggaccaagctggagctgaaac

SEQ ID NO: 359 - DNA, 367 bp, synthetic construct
  misc_feature 1..367: note = Synthetic polynucleotide
  source 1..367: mol_type = other DNA
caggtccagctgcagcagtctggagctgagctggtaaggcctgggacttcagtgaaggtgtcctgcaaggcttctggatacgccttcagtaattacttgatagagtgggttaagcagaggcctggacagggccttgagtggattggagtgattaatcctggaagtggtggtactaactacaatgagaagttcaagggcaaggcaacaatgactgcagacaaatcctccagcactgcctacatgcacctcagcaacctgacatctgaggactctgtggtctatttctgtgcaagatcatacttcggtagaagctacccctatactatggactactggggtcaaggaacctcagtcaccgtctcctcag

SEQ ID NO: 360 - DNA, 334 bp, synthetic construct
  misc_feature 1..334: note = Synthetic polynucleotide
  source 1..334: mol_type = other DNA
gatattgtgctgacccaatctccagcttctttggctgtgtctctagggcagagggccaccatctcctgcaaggccagccaaagtgttgattatgatggtgataattatgtgaactggtaccaacagaaagtaggacagccacccaaactcctcatctctgctgcatccaatctagaatctgggatcccagccaggtttagtggcagtgggtctgggacagacttcaccctcaacatccatcctgtggaggaggaggatgctgcaacctattactgtcagcaaagtaatgaggatccattcacgttcggctcggggacaaagttggaaataaaac

SEQ ID NO: 361 - DNA, 361 bp, synthetic construct
  misc_feature 1..361: note = Synthetic polynucleotide
  source 1..361: mol_type = other DNA
caggtccagctgcagcagtctgggcctgagctggtgaggcctggggtctcagtgaagatttcctgcaagggttccggctacacattcactgattatgctatgcactgggtgaagcagagtcatgcaaagagtctagagtggattggagttattagtacatactctggtaatacaaactacaaccagaagtttcaggacaaggccaccatgactgtagacaaatcctccagcacagcctatatggcacttgccagattgacatctgacgattctgccatctattactgtgcaagaaggggcgattacagcctctatgctatggactactggggtcaaggaacctcagtcaccgtctcctcag

SEQ ID NO: 362 - DNA, 337 bp, synthetic construct
  misc_feature 1..337: note = Synthetic polynucleotide
  source 1..337: mol_type = other DNA
aacattatgatgacacagtcgccatcatctctggctgtgtctgcaggagaaaaggtcactatgagctgtaagtccagtcaaagtgttttattcagttcaaatcagaaaaactacttggcctggtaccagcagaaaccagggcagtctcctagactgctgatctactgggcatccactagggaatctggtgtccctgatcgcttcacaggcagtggatctgggacagattttactcttaccatcagcaatgttcaagctgaagacctggcagtttattactgtcatcaatacctctcctcgcgcacgttcggtgctgggaccaagctggagctgaaac

SEQ ID NO: 363 - DNA, 355 bp, synthetic construct; identical to SEQ ID NO: 355
  misc_feature 1..355: note = Synthetic polynucleotide
  source 1..355: mol_type = other DNA

SEQ ID NO: 364 - DNA, 334 bp, synthetic construct
  misc_feature 1..334: note = Synthetic polynucleotide
  source 1..334: mol_type = other DNA
gacattgtgctgacccaatctccagcttctttggctctgtctctagggcagagggccaccatctcctgcagagccagcgaaagtgttgataattatggcattagttttatgaactggttccaacagaaacccggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgctcacgttcggtgctgggaccaagctggagctgaaac

SEQ ID NO: 365 - DNA, 358 bp, synthetic construct
  misc_feature 1..358: note = Synthetic polynucleotide
  source 1..358: mol_type = other DNA
gaagtgaggctggtggagtctgggggaggcttgatgcagcctggagggtccctgaaactcccctgtgcagcctctggattccctttcagtagctctgccatgtcttgggttcgccagactccagagaagaggctggagtgggtcgcatccattaatagtgatggtaacacctactatcccgacagtgtgaagggccgattcaccatctccagagatagtgccaggaacatcctgtacctccaaatgagcagtctgaggtctgaggacacggccatgtattactgtacaagaaacggggactataggtacgacgagtttgcttactggggccaagggactctggtcactgtctctgcag

SEQ ID NO: 366 - DNA, 325 bp, synthetic construct
  misc_feature 1..325: note = Synthetic polynucleotide
  source 1..325: mol_type = other DNA
caaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccacgttcggagctgggaccaagctggagctgaaac

SEQ ID NO: 367 - DNA, 358 bp, synthetic construct
  misc_feature 1..358: note = Synthetic polynucleotide
  source 1..358: mol_type = other DNA
caggtccagcttcagcagtctggggctgaactggcaaaacctggggcctcagtgaagatgtcctgcaaggcttctggctacacctttactggctactggatgcactgggtaaaacagaggcctggacagggtctggaatggcttggatacattaatcctagcactggttatactgagtccaatcagaagttcaaggacaaggccacattgactgcagacaaatcctccaccacagcctacatgcaactgagaagcctgacacctgaggactctgcagtctattactgtgcaagagaggggattactactgtgctggttgactactggggccaaggcaccactctcacagtctcctcag

SEQ ID NO: 368 - DNA, 337 bp, synthetic construct; identical to SEQ ID NO: 362
  misc_feature 1..337: note = Synthetic polynucleotide
  source 1..337: mol_type = other DNA

SEQ ID NO: 369 - DNA, 364 bp, synthetic construct
  misc_feature 1..364: note = Synthetic polynucleotide
  source 1..364: mol_type = other DNA
caggtgcagctgaagcagtctggacctgagctggagaagcctggcgcttcagtgaagatatcctgcaaggcttctggttactctttcactggctacaatatgaactgggtgaagcagagcaatggaaagagccttgagtggattggaaatattgatccttactatggtggttctacctacaaccagaagttcacgggcaaggccacattgactgtagacaaatcctccagcacagcctacatgcagctcaagagcctgacatctgaggactctgcagtgtattactgtgcaagagagaggtcgggctacgttttctctgctatggactactggggtcaaggaacctcagtcaccgtctcctcag

SEQ ID NO: 370 - DNA, 337 bp, synthetic construct; identical to SEQ ID NO: 362
  misc_feature 1..337: note = Synthetic polynucleotide
  source 1..337: mol_type = other DNA

SEQ ID NO: 371 - DNA, 370 bp, synthetic construct
  misc_feature 1..370: note = Synthetic polynucleotide
  source 1..370: mol_type = other DNA
gaggtccagctgcaacagtctggacctgagctggtgaagcctggaggttcaatgaagatatcctgcaaggcttctggttactcattcactggctacaccatgaactgggtgaagcggagccatggaaagaaccttgagtggattggacttattaatccttacaatggtgttactacctacaaccagaacttcaagggcaaggccacattagctgtagacaagtcatccagcacagcctacatggagctcctcggtctgacatctgaggactctgcagtctattactgtacaagagatcccctttactacggctacagggactctactatggactactggggtcaaggaacctcagtcaccgtctcctcag

SEQ ID NO: 372 - DNA, 337 bp, synthetic construct; identical to SEQ ID NO: 340
  misc_feature 1..337: note = Synthetic polynucleotide
  source 1..337: mol_type = other DNA

SEQ ID NO: 373 - DNA, 352 bp, synthetic construct
  misc_feature 1..352: note = Synthetic polynucleotide
  source 1..352: mol_type = other DNA
gaggtccagctgcagcagtctggggctgaactggcaagacctggggcctcagtgaagatgtcctgcaaggcttctggctacacctttactagctacacgatgcactggataaaacagagacctggacagggtcaggaatggattggatacattaatcctagcagtacgtatactcattacattaagaagttcaaggacaaggccacattgactgcagacaaatcctccagcacagcctacatgcaactgcgcagcctgacatctgaggactctgcagtctattactgttcaagaggggaactgggagggtttgcttactggggccaagggactctggtcactgtctctgcag

SEQ ID NO: 374 - DNA, 322 bp, synthetic construct
  misc_feature 1..322: note = Synthetic polynucleotide
  source 1..322: mol_type = other DNA
gacatccagatgattcagtctccatcgtccatgtttgcctctctgggagacagagtcagtctctcttgtcgggctagtcagggcattagaggtaatttagactggtatcagcagaaaccaggtggaactattaaactcctgatctactccacatccattttaaattctggtgtcccatcaaggttcagtggcagtgggtctgggtcagattattctctcaccatcaccagcctagagtctgaagattttgcagactattactgtctacagcgtaatgcgtatcctctcacgttcggttctgggaccaagctggagctgaaac

SEQ ID NO: 375 - DNA, 358 bp, synthetic construct
  misc_feature 1..358: note = Synthetic polynucleotide
  source 1..358: mol_type = other DNA
gaagtgaagttggtggagtctgggggaggcttaatgaagcctggagggtccctgaaactctcctgtgcggcctctggattcactttcagtagttttgccttgtcttgggttcgccagactccagagaagaggctggagtgggtcgcatccattagaagtggtggtattacctaccatgcagacagtgtgaagggccgattcaccatctccagagataatgccgggaacatcctgtacctgcaaatgaacagtctgaggtctgaggacacggccatgtatttctgtgcaagagttagtacggctacgtactatggtatggactactggggtcaaggaacctcagtcaccgtctcctcag

SEQ ID NO: 376 - protein, 116 aa, synthetic construct
  REGION 1..116: note = Synthetic peptide
QVQLVQSGAEVKKPGASVKVSCKASGYTFTTYTMHWVRQAPGQGLEWMGFINPSSGYTDYAQKFQGRVTMTRDTSTSTVYMELSSLRSEDTAVYYCARGDYYVAYWGQGTLVTVSS

SEQ ID NO: 377 - protein, 116 aa, synthetic construct
  REGION 1..116: note = Synthetic peptide
QVQLVQSGAEVKKPGASVKVSCKASGYTFTTYTMHWVRQAPGQGLEWMGFINPSSGYTDYAQKFQGRVTMTADTSTSTVYMELSSLRSEDTAVYYCANGDYYVAYWGQGTLVTVSS

SEQ ID NO: 378 - protein, 116 aa, synthetic construct
  REGION 1..116: note = Synthetic peptide
QVQLVQSGAEVKKPGASVKMSCKASGYTFTTYTMHWVRQAPGQGLEWIGFINPSSGYTDYAQKFQGRVTLTADTSTSTVYMELSSLRSEDTAVYYCANGDYYVAYWGQGTLVTVSS

SEQ ID NO: 379 - protein, 116 aa, synthetic construct
  REGION 1..116: note = Synthetic peptide
QVQLVQSGAEVKKPGASVKMSCKASGYTFTTYTMHWVRQAPGQGLEWIGFINPSSGYTDYAQKFQGRVTLTADKSTSTVYMELSSLRSEDTAVYYCANGDYYVAYWGQGTLVTVSS

SEQ ID NO: 380 - protein, 108 aa, synthetic construct
  REGION 1..108: note = Synthetic peptide
DIQMTQSPSSLSASVGDRVTITCTASSSVSSTYFHWYQQKPGKAPKLLIYSTSNLASGVPSRFSGSGSGTDFTLTISSLQPEDFATYYCHQYHRSPLTFGQGTKLEIK

SEQ ID NO: 381 - protein, 108 aa, synthetic construct
  REGION 1..108: note = Synthetic peptide
DIQMTQSPSSLSASVGDRVTITCTASSSVSSTYFHWYQQKPGKAPKLLIYSTSNLASGVPSRFSGSGSGTDYTLTISSLQPEDFATYYCHQYHRSPLTFGQGTKLEIK

SEQ ID NO: 382 - protein, 108 aa, synthetic construct
  REGION 1..108: note = Synthetic peptide
DIQLTQSPSSLSASVGDRVTMTCTASSSVSSTYFHWYQQKPGKAPKLLIYSTSNLASGVPSRFSGSGSGTDYTLTISSMQPEDAATYYCHQYHRSPLTFGQGTKLEIK

SEQ ID NO: 383 - protein, 108 aa, synthetic construct
  REGION 1..108: note = Synthetic peptide
DIQLTQSPSSLSASVGDRVTMTCTASSSVSSTYFHWYQQKPGKAPKLWIYSTSNLASGVPSRFSGSGSGTDYTLTISSMQPEDAATYYCHQYHRSPLTFGQGTKLEIK

SEQ ID NO: 384 - protein, 117 aa, synthetic construct
  REGION 1..117: note = Synthetic peptide
EVQLLESGGGLVQPGGSLRLSCAASGFTFSNYWMNWVRQAPGKGLEWVSQIRLKSDNYATHYADSVKGRFTISRDNSKNTLYLQMNSLRAEDTAVYYCAKGPPSGCWGQGTLVTVSS

SEQ ID NO: 385 - protein, 117 aa, synthetic construct
  REGION 1..117: note = Synthetic peptide
EVQLLESGGGLVQPGGSMRLSCAASGFTFSNYWMNWVRQAPGKGLEWVAQIRLKSDNYATHYADSVKGRFTISRDDSKNTVYLQMNSLRAEDTGVYYCNDGPPSGCWGQGTLLTVSS

SEQ ID NO: 386 - protein, 117 aa, synthetic construct
  REGION 1..117: note = Synthetic peptide
EVQLLESGGGLVQPGGSMRLSCAASGFTFSNYWMNWVRQAPGKGLEWVAQIRLKSDNYATHYADSVKGRFTISRDDSKNTVYLQMNSLRAEDTGVYYCNDGPPSGYWGQGTLLTVSS

SEQ ID NO: 387 - protein, 117 aa, synthetic construct
  REGION 1..117: note = Synthetic peptide
EVQLLESGGGLVQPGGSMRLSCAASGFTFSNYWMNWVRQAPGKGLEWVAQIRLKSDNYATHYADSVKGRFTISRDDSKNTVYLQMNSLRAEDTGVYYCNDGPPSGSWGQGTLLTVSS

SEQ ID NO: 388 - protein, 107 aa, synthetic construct
  REGION 1..107: note = Synthetic peptide
DIQMTQSPSSLSASVGDRVTITCRISENIYSYLAWYQQKPGKAPKLLIYNAKILVEGVPSRFSGSGSGTDFTLTISSLQPEDFATYYCQHHYTVPWTFGQGTKLEIK

SEQ ID NO: 389 - protein, 107 aa, synthetic construct
  REGION 1..107: note = Synthetic peptide
DIQMTQSPSSLSASVGDRVTITCRISENIYSYLAWYQQKPGKAPKLLVYNAKILVEGVPSRFSGSGSGTDFTLTISSLQPEDFGTYYCQHHYTVPWTFGQGTKLEIK

SEQ ID NO: 390 - protein, 107 aa, synthetic construct
  REGION 1..107: note = Synthetic peptide
DIQMTQSPSSLSASVGDRVTITCRISENIYSYLAWYQQKPGKAPKLLVYNAKSLVEGVPSRFSGSGSGTDFTLTISSLQPEDFGTYYCQHHYTVPWTFGQGTKLEIK

SEQ ID NO: 391 - protein, 117 aa, synthetic construct
  REGION 1..117: note = Synthetic peptide
QVQLVQSGAEVKKPGASVKVSCKASGYTFTSYTMHWVRQAPGQGLEWMGYINPSSTYTHYAQKFQGRVTMTRDTSTSTVYMELSSLRSEDTAVYYCARGELGGFAYWGQGTLVTVSS

SEQ ID NO: 392 - protein, 117 aa, synthetic construct
  REGION 1..117: note = Synthetic peptide
QVQLVQSGAEVKKPGASVKVSCKASGYTFTSYTMHWVRQAPGQGLEWMGYINPSSTYTHYAQKFQGRVTMTADTSTSTVYMELSSLRSEDTAVYYCSRGELGGFAYWGQGTLVTVSS

SEQ ID NO: 393 - protein, 117 aa, synthetic construct
  REGION 1..117: note = Synthetic peptide
QVQLVQSGAEVKKPGASVKMSCKASGYTFTSYTMHWIRQAPGQGLEWIGYINPSSTYTHYAQKFQGRATLTADTSTSTAYMELSSLRSEDTAVYYCSRGELGGFAYWGQGTLVTVSS

SEQ ID NO: 394 - protein, 117 aa, synthetic construct
  REGION 1..117: note = Synthetic peptide
QVQLVQSGAEVKKPGASVKMSCKASGYTFTSYTMHWIRQAPGQGQEWIGYINPSSTYTHYAQKFQGRATLTADTSTSTAYMELSSLRSEDTAVYYCSRGELGGFAYWGQGTLVTVSS

SEQ ID NO: 395 - protein, 117 aa, synthetic construct
  REGION 1..117: note = Synthetic peptide
QVQLVQSGAEVKKPGASVKMSCKASGYTFTSYTMHWIRQAPGQGLEWIGYINPSSTYTHYAQKFQGRATLTADKSTSTAYMELSSLRSEDTAVYYCSRGELGGFAYWGQGTLVTVSS

SEQ ID NO: 396 - protein, 117 aa, synthetic construct
  REGION 1..117: note = Synthetic peptide
QVQLVQSGAEVKKPGASVKMSCKASGYTFTSYTMHWIRQAPGQGQEWIGYINPSSTYTHYAQKFQGRATLTADKSTSTAYMELSSLRSEDTAVYYCSRGELGGFAYWGQGTLVTVSS

SEQ ID NO: 397 - protein, 112 aa, synthetic construct
  REGION 1..112: note = Synthetic peptide
DVVMTQSPLSLPVTLGQPASISCRSSQSLVHSDGNTYLNWFQQRPGQSPRRLIYKVSNRFSGVPDRFSGSGSGTDFTLKISRVEAEDVGVYYCSQSTHVPYTFGQGTKLEIK

SEQ ID NO: 398 - protein, 112 aa, synthetic construct
  REGION 1..112: note = Synthetic peptide
DVVMTQSPLSLPVTLGQPASISCRSSQSLVHSDGNTYLNWYQQRPGQSPRLLIYKVSNRFSGVPDRFSGSGSGTDFTLKISRVEAEDVGVYYCSQSTHVPYTFGQGTKLEIK

SEQ ID NO: 399 - protein, 112 aa, synthetic construct
  REGION 1..112: note = Synthetic peptide
DVVMTQSPLSLPVTLGQPASISCRSSQSLVHSEGNTYLNWYQQRPGQSPRLLIYKVSNRFSGVPDRFSGSGSGTDFTLKISRVEAEDVGVYYCSQSTHVPYTFGQGTKLEIK

SEQ ID NO: 400 - protein, 112 aa, synthetic construct
  REGION 1..112: note = Synthetic peptide
DVVMTQSPLSLPVTLGQPASISCRSSQSLVHSSGNTYLNWYQQRPGQSPRLLIYKVSNRFSGVPDRFSGSGSGTDFTLKISRVEAEDVGVYYCSQSTHVPYTFGQGTKLEIK

SEQ ID NO: 401 - protein, 112 aa, synthetic construct
  REGION 1..112: note = Synthetic peptide
DVVMTQSPLSLPVTLGQPASISCRSSQSLVHSEGSTYLNWYQQRPGQSPRLLIYKVSNRFSGVPDRFSGSGSGTDFTLKISRVEAEDVGVYYCSQSTHVPYTFGQGTKLEIK

SEQ ID NO: 402 - protein, 112 aa, synthetic construct
  REGION 1..112: note = Synthetic peptide
DVVMTQSPLSLPVTLGQPASISCRSSQSLVHSSGSTYLNWYQQRPGQSPRLLIYKVSNRFSGVPDRFSGSGSGTDFTLKISRVEAEDVGVYYCSQSTHVPYTFGQGTKLEIK

SEQ ID NO: 403 - protein, 117 aa, synthetic construct
  REGION 1..117: note = Synthetic peptide
QVQLVQSGAEVKKPGASVKVSCKASGYRFTDYNMHWVRQAPGQGLEWMGYINPNNGGTNYAQKFQGRVTMTRDTSTSTVYMELSSLRSEDTAVYYCARDYLYFFDCWGQGTLVTVSS

SEQ ID NO: 404 - protein, 117 aa, synthetic construct
  REGION 1..117: note = Synthetic peptide
QVQLVQSGAEVKKPGASVKVSCKASGYRFTDYNMHWVRQAPGQGLEWMGYINPNNGGTNYAQKFQGRVTMTVDTSTSTVYMELSSLRSEDTAVYYCARDYLYFFDCWGQGTLVTVSS

SEQ ID NO: 405 - protein, 117 aa, synthetic construct
  REGION 1..117: note = Synthetic peptide
QVQLVQSGAEVKKPGASVKMSCKASGYRFTDYNMHWVRQAPGQGLEWIGYINPNNGGTNYAQKFQGRATLTVDTSTSTAYMELSSLRSEDTAVYYCARDYLYFFDCWGQGTLLTVSS

SEQ ID NO: 406 - protein, 117 aa, synthetic construct
  REGION 1..117: note = Synthetic peptide
QVQLVQSGAEVKKPGASVKMSCKASGYRFTDYNMHWVRQAPGQGLEWIGYINPNNGGTNYAQKFQGRATLTVDKSTSTAYMELSSLRSEDTAVYYCARDYLYFFDCWGQGTLLTVSS

SEQ ID NO: 407 - protein, 117 aa, synthetic construct
  REGION 1..117: note = Synthetic peptide
QVQLVQSGAEVKKPGASVKMSCKASGYRFTDYNMHWVRQAPGQGLEWIGYINPNNGGTNYAQKFQGRATLTVNKSTSTAYMELSSLRSEDTAVYYCARDYLYFFDCWGQGTLLTVSS

SEQ ID NO: 408 - protein, 117 aa, synthetic construct
  REGION 1..117: note = Synthetic peptide
QVQLVQSGAEVKKPGASVKMSCKASGYRFTDYNMHWVRQAPGQGLEWIGYINPNNGGTNYAQKFQGRATLTVNTSTSTAYMELSSLRSEDTAVYYCARDYLYFFDCWGQGTLLTVSS

SEQ ID NO: 409 - protein, 107 aa, synthetic construct
  REGION 1..107: note = Synthetic peptide
DIQMTQSPSSLSASVGDRVTITCLASQTIGTWLAWYQQKPGKAPKLLIYAAASLADGVPSRFSGSGSGTDFTLTISSLQPEDFATYYCQQLYSIPRTFGQGTKLEIK

SEQ ID NO: 410 - protein, 107 aa, synthetic construct
  REGION 1..107: note = Synthetic peptide
DIQMTQSPSSLSASVGDRVTITCLASQTIGTWLAWYQQKPGKAPKLLIYAAASLADGVPSRFSGSGSGTDFTFTISSLQPEDFVTYYCQQLYSIPRTFGQGTKLEIK

SEQ ID NO: 411 - protein, 107 aa, synthetic construct
  REGION 1..107: note = Synthetic peptide
DIQMTQSPSSLSASVGDRVTITCRASQTIGTWLAWYQQKPGKAPKLLIYAAASLADGVPSRFSGSGSGTDFTFTISSLQPEDFVTYYCQQLYSIPRTFGQGTKLEIK

SEQ ID NO: 412 - protein, 118 aa, synthetic construct
  REGION 1..118: note = Synthetic peptide
EVQLVESGGGLVQPGGSLRLSCAASGFTFSSFGMHWVRQAPGKGLEWVSYISSDSRTIYYADSVKGRFTISRDNAKNSLYLQMNSLRAEDTAVYYCARDYGRTYEAYWGQGTLVTVSS

SEQ ID NO: 413 - protein, 118 aa, synthetic construct
  REGION 1..118: note = Synthetic peptide
EVQLVESGGGLVQPGGSLRLSCAASGFTFSSFGMHWVRQAPGKGLEWVAYISSDSRTIYYADSVKGRFTISRDNAKNSLYLQMNSLRAEDTAVYYCARDYGRTYEAYWGQGTLVTVSS

SEQ ID NO: 414 - protein, 111 aa, synthetic construct
  REGION 1..111: note = Synthetic peptide
DIVMTQSPDSLAVSLGERATINCRASKSVSTSGYSYIHWYQQKPGQPPKLLIYLASNLESGVPDRFSGSGSGTDFTLTISSLQAEDVAVYYCQHSRELPLTFGQGTKLEIK

SEQ ID NO: 415 - protein, 111 aa, synthetic construct
  REGION 1..111: note = Synthetic peptide
DIVLTQSPDSLAVSLGERATINCRASKSVSTSGYSYIHWYQQKPGQPPKLLIYLASNLESGVPDRFSGSGSGTDFTLTISSVQAEDVAVYYCQHSRELPLTFGQGTKLELK

SEQ ID NO: 416 - protein, 121 aa, synthetic construct
  REGION 1..121: note = Synthetic peptide
QVQLVQSGAEVKKPGASVKVSCKASGYSFTGYNMNWVRQAPGQGLEWMGNIDPYYGGSTYAQKFQGRVTMTRDTSTSTVYMELSSLRSEDTAVYYCARERSGYVFSAMDYWGQGTLVTVSS

SEQ ID NO: 417 - protein, 121 aa, synthetic construct
  REGION 1..121: note = Synthetic peptide
QVQLVQSGAEVKKPGASVKVSCKASGYSFTGYNMNWVRQAPGQGLEWMGNIDPYYGGSTYAQKFQGRVTMTVDTSTSTVYMELSSLRSEDTAVYYCARERSGYVFSAMDYWGQGTLVTVSS

SEQ ID NO: 418 - protein, 121 aa, synthetic construct
  REGION 1..121: note = Synthetic peptide
QVQLVQSGAEVKKPGASVKISCKASGYSFTGYNMNWVRQAPGQGLEWIGNIDPYYGGSTYAQKFQGRATLTVDTSTSTAYMELSSLRSEDTAVYYCARERSGYVFSAMDYWGQGTLVTVSS

SEQ ID NO: 419 - protein, 121 aa, synthetic construct
  REGION 1..121: note = Synthetic peptide
QVQLVQSGAEVKKPGASVKISCKASGYSFTGYNMNWVRQAPGQGLEWIGNIDPYYGGSTYAQKFQGRATLTVDKSTSTAYMELSSLRSEDTAVYYCARERSGYVFSAMDYWGQGTLVTVSS

SEQ ID NO: 420 - protein, 112 aa, synthetic construct
  REGION 1..112: note = Synthetic peptide
DIVMTQSPDSLAVSLGERATINCKSSQSVLFSSNQKNYLAWYQQKPGQPPKLLIYWASTRESGVPDRFSGSGSGTDFTLTISSLQAEDVAVYYCHQYLSSRTFGQGTKLEIK

SEQ ID NO: 421 - protein, 112 aa, synthetic construct
  REGION 1..112: note = Synthetic peptide
DIVMTQSPDSLAVSLGERVTMNCKSSQSVLFSSNQKNYLAWYQQKPGQPPKLLIYWASTRESGVPDRFSGSGSGTDFTLTISSVQAEDVAVYYCHQYLSSRTFGQGTKLEIK

SEQ ID NO: 422 - protein, 117 aa, synthetic construct
  REGION 1..117: note = Synthetic peptide
EVQLLESGGGLVQPGGSMRLSCAASGFTFSNYWMNWVRQAPGKGLEWVAQIRLKSDNYATHYADSVKGRFTISRDDSKNTVYLQMNSLRAEDTGVYYCNDGPPSGAWGQGTLLTVSS

SEQ ID NO: 423 - protein, 117 aa, synthetic construct
  REGION 1..117: note = Synthetic peptide
QVQLVQSGAEVKKPGASVKVSCKASGYTFTSYTMHWVRQAPGQGLEWMGYINPSSTYTHYIKKFKDRVTMTADTSTSTVYMELSSLRSEDTAVYYCSRGELGGFAYWGQGTLVTVSS

SEQ ID NO: 424 - protein, 117 aa, synthetic construct
  REGION 1..117: note = Synthetic peptide
QVQLVQSGAEVKKPGASVKMSCKASGYTFTSYTMHWIRQAPGQGLEWIGYINPSSTYTHYIKKFKDRATLTADTSTSTAYMELSSLRSEDTAVYYCSRGELGGFAYWGQGTLVTVSS

SEQ ID NO: 425 - protein, 117 aa, synthetic construct
  REGION 1..117: note = Synthetic peptide
QVQLVQSGAEVKKPGASVKMSCKASGYTFTSYTMHWIRQAPGQGQEWIGYINPSSTYTHYIKKFKDRATLTADTSTSTAYMELSSLRSEDTAVYYCSRGELGGFAYWGQGTLVTVSS

SEQ ID NO: 426 - protein, 117 aa, synthetic construct
  REGION 1..117: note = Synthetic peptide
QVQLVQSGAEVKKPGASVKMSCKASGYTFTSYTMHWIRQAPGQGLEWIGYINPSSTYTHYIKKFKDRATLTADKSTSTAYMELSSLRSEDTAVYYCSRGELGGFAYWGQGTLVTVSS

SEQ ID NO: 427 - protein, 117 aa, synthetic construct
  REGION 1..117: note = Synthetic peptide
QVQLVQSGAEVKKPGASVKMSCKASGYTFTSYTMHWIRQAPGQGQEWIGYINPSSTYTHYIKKFKDRATLTADKSTSTAYMELSSLRSEDTAVYYCSRGELGGFAYWGQGTLVTVSS

SEQ ID NO: 428 - protein, 116 aa, synthetic construct
  REGION 1..116: note = Synthetic peptide
  VARIANT 5: note = Xaa can be any naturally occurring amino acid
  VARIANT 8: note = Xaa can be any naturally occurring amino acid
  VARIANT 11..13: note = Xaa can be any naturally occurring amino acid
  VARIANT 20: note = Xaa can be any naturally occurring amino acid
  VARIANT 31: note = Xaa can be any naturally occurring amino acid
  VARIANT 33: note = Xaa can be any naturally occurring amino acid
  VARIANT 35: note = Xaa can be any naturally occurring amino acid
  VARIANT 38: note = Xaa can be any naturally occurring amino acid
  VARIANT 40: note = Xaa can be any naturally occurring amino acid
  VARIANT 48: note = Xaa can be any naturally occurring amino acid
  VARIANT 50: note = Xaa can be any naturally occurring amino acid
  VARIANT 55: note = Xaa can be any naturally occurring amino acid
  VARIANT 57: note = Xaa can be any naturally occurring amino acid
  VARIANT 59: note = Xaa can be any naturally occurring amino acid
  VARIANT 61: note = Xaa can be any naturally occurring amino acid
  VARIANT 65..68: note = Xaa can be any naturally occurring amino acid
  VARIANT 70: note = Xaa can be any naturally occurring amino acid
  VARIANT 72: note = Xaa can be any naturally occurring amino acid
  VARIANT 74..76: note = Xaa can be any naturally occurring amino acid
  VARIANT 79..80: note = Xaa can be any naturally occurring amino acid
  VARIANT 82: note = Xaa can be any naturally occurring amino acid
  VARIANT 87: note = Xaa can be any naturally occurring amino acid
  VARIANT 90..91: note = Xaa can be any naturally occurring amino acid
  VARIANT 98: note = Xaa can be any naturally occurring amino acid
  VARIANT 101: note = Xaa can be any naturally occurring amino acid
  VARIANT 116: note = Xaa can be any naturally occurring amino acid
QVQLXQSXAEXXXPGASVKXSCKASGYTFTXYXMXWVXQXPGQGLEWXGXINPSXGXTXYXQKFXXXXTXTXDXSXSTXXMXLSSLXSEXXAVYYCAXGDXYVAYWGQGTLVTVSX

SEQ ID NO: 429 - protein, 116 aa, synthetic construct
  REGION 1..116: note = Synthetic peptide
  VARIANT 20: note = Xaa can be any naturally occurring amino acid
  VARIANT 31: note = Xaa can be any naturally occurring amino acid
  VARIANT 35: note = Xaa can be any naturally occurring amino acid
  VARIANT 48: note = Xaa can be any naturally occurring amino acid
  VARIANT 50: note = Xaa can be any naturally occurring amino acid
  VARIANT 59: note = Xaa can be any naturally occurring amino acid
  VARIANT 67: note = Xaa can be any naturally occurring amino acid
  VARIANT 70: note = Xaa can be any naturally occurring amino acid
  VARIANT 74: note = Xaa can be any naturally occurring amino acid
  VARIANT 79: note = Xaa can be any naturally occurring amino acid
  VARIANT 98: note = Xaa can be any naturally occurring amino acid
  VARIANT 101: note = Xaa can be any naturally occurring amino acid
QVQLVQSGAEVKKPGASVKXSCKASGYTFTXYTMXWVRQAPGQGLEWXGXINPSSGYTXYAQKFQGXVTXTADXSTSTXYMELSSLRSEDTAVYYCAXGDXYVAYWGQGTLVTVSS

SEQ ID NO: 430 - protein, 108 aa, synthetic construct
  REGION 1..108: note = Synthetic peptide
  VARIANT 1: note = Xaa can be any naturally occurring amino acid
  VARIANT 3..4: note = Xaa can be any naturally occurring amino acid
  VARIANT 9..11: note = Xaa can be any naturally occurring amino acid
  VARIANT 15: note = Xaa can be any naturally occurring amino acid
  VARIANT 17: note = Xaa can be any naturally occurring amino acid
  VARIANT 21: note = Xaa can be any naturally occurring amino acid
  VARIANT 24: note = Xaa can be any naturally occurring amino acid
  VARIANT 27: note = Xaa can be any naturally occurring amino acid
  VARIANT 29: note = Xaa can be any naturally occurring amino acid
  VARIANT 32: note = Xaa can be any naturally occurring amino acid
  VARIANT 34..35: note = Xaa can be any naturally occurring amino acid
  VARIANT 43..44: note = Xaa can be any naturally occurring amino acid
  VARIANT 48: note = Xaa can be any naturally occurring amino acid
  VARIANT 51..56: note = Xaa can be any naturally occurring amino acid
  VARIANT 61: note = Xaa can be any naturally occurring amino acid
  VARIANT 67: note = Xaa can be any naturally occurring amino acid
  VARIANT 71..73: note = Xaa can be any naturally occurring amino acid
  VARIANT 79..81: note = Xaa can be any naturally occurring amino acid
  VARIANT 84: note = Xaa can be any naturally occurring amino acid
  VARIANT 90..91: note = Xaa can be any naturally occurring amino acid
  VARIANT 93..96: note = Xaa can be any naturally occurring amino acid
  VARIANT 101: note = Xaa can be any naturally occurring amino acid
  VARIANT 107: note = Xaa can be any naturally occurring amino acid
XIXXTQSPXXXSASXGXRVTXTCXASXSXSSXYXXWYQQKPGXXPKLXIYXXXXXXSGVPXRFSGSXSGTXXXLTISSXXXEDXATYYCXXYXXXXLTFGXGTKLEXK

SEQ ID NO: 431 - protein, 108 aa, synthetic construct
  REGION 1..108: note = Synthetic peptide
  VARIANT 4: note = Xaa can be any naturally occurring amino acid
  VARIANT 21: note = Xaa can be any naturally occurring amino acid
  VARIANT 32: note = Xaa can be any naturally occurring amino acid
  VARIANT 34: note = Xaa can be any naturally occurring amino acid
  VARIANT 48: note = Xaa can be any naturally occurring amino acid
  VARIANT 51: note = Xaa can be any naturally occurring amino acid
  VARIANT 53: note = Xaa can be any naturally occurring amino acid
  VARIANT 61: note = Xaa can be any naturally occurring amino acid
  VARIANT 67: note = Xaa can be any naturally occurring amino acid
  VARIANT 79: note = Xaa can be any naturally occurring amino acid
  VARIANT 84: note = Xaa can be any naturally occurring amino acid
  VARIANT 91: note = Xaa can be any naturally occurring amino acid
  VARIANT 93: note = Xaa can be any naturally occurring amino acid
DIQXTQSPSSLSASVGDRVTXTCTASSSVSSXYXHWYQQKPGKAPKLXIYXTXNLASGVPXRFSGSXSGTDYTLTISSXQPEDXATYYCHXYXRSPLTFGQGTKLEIK

SEQ ID NO: 432 - protein, 117 aa, synthetic construct
  REGION 1..117: note = Synthetic peptide
  VARIANT 3: note = Xaa can be any naturally occurring amino acid
  VARIANT 5: note = Xaa can be any naturally occurring amino acid
  VARIANT 18..19: note = Xaa can be any naturally occurring amino acid
  VARIANT 23: note = Xaa can be any naturally occurring amino acid
  VARIANT 31: note = Xaa can be any naturally occurring amino acid
  VARIANT 33: note = Xaa can be any naturally occurring amino acid
  VARIANT 35: note = Xaa can be any naturally occurring amino acid
  VARIANT 40: note = Xaa can be any naturally occurring amino acid
  VARIANT 42: note = Xaa can be any naturally occurring amino acid
  VARIANT 49..50: note = Xaa can be any naturally occurring amino acid
  VARIANT 52..59: note = Xaa can be any naturally occurring amino acid
  VARIANT 61: note = Xaa can be any naturally occurring amino acid
  VARIANT 64: note = Xaa can be any naturally occurring amino acid
  VARIANT 76: note = Xaa can be any naturally occurring amino acid
  VARIANT 79..81: note = Xaa can be any naturally occurring amino acid
  VARIANT 87: note = Xaa can be any naturally occurring amino acid
  VARIANT 94..95: note = Xaa can be any naturally occurring amino acid
  VARIANT 99: note = Xaa can be any naturally occurring amino acid
  VARIANT 106: note = Xaa can be any naturally occurring amino acid
  VARIANT 112: note = Xaa can be any naturally occurring amino acid
  VARIANT 114: note = Xaa can be any naturally occurring amino acid
EVXLXESGGGLVQPGGSXXLSCXASGFTFSXYXMXWVRQXPXKGLEWVXXIXXXXXXXXTXYAXSVKGRFTISRDXSKXXXYLQMNXLRAEDTXXYYCXDGPPSGXWGQGTXLXVSS

SEQ ID NO: 433 - protein, 117 aa, synthetic construct
  REGION 1..117: note = Synthetic peptide
  VARIANT 31: note = Xaa can be any naturally occurring amino acid
  VARIANT 35: note = Xaa can be any naturally occurring amino acid
  VARIANT 50: note = Xaa can be any naturally occurring amino acid
  VARIANT 55: note = Xaa can be any naturally occurring amino acid
  VARIANT 79: note = Xaa can be any naturally occurring amino acid
  VARIANT 99: note = Xaa can be any naturally occurring amino acid
  VARIANT 106: note = Xaa can be any naturally occurring amino acid
EVQLLESGGGLVQPGGSMRLSCAASGFTFSXYWMXWVRQAPGKGLEWVAXIRLKXDNYATHYADSVKGRFTISRDDSKXTVYLQMNSLRAEDTGVYYCXDGPPSGXWGQGTLLTVSS

SEQ ID NO: 434 - protein, 107 aa, synthetic construct
  REGION 1..107: note = Synthetic peptide
  VARIANT 9: note = Xaa can be any naturally occurring amino acid
  VARIANT 17..18: note = Xaa can be any naturally occurring amino acid
  VARIANT 25: note = Xaa can be any naturally occurring amino acid
  VARIANT 27..28: note = Xaa can be any naturally occurring amino acid
  VARIANT 30: note = Xaa can be any naturally occurring amino acid
  VARIANT 34: note = Xaa can be any naturally occurring amino acid
  VARIANT 40: note = Xaa can be any naturally occurring amino acid
  VARIANT 43: note = Xaa can be any naturally occurring amino acid
  VARIANT 45: note = Xaa can be any naturally occurring amino acid
  VARIANT 48: note = Xaa can be any naturally occurring amino acid
  VARIANT 50: note = Xaa can be any naturally occurring amino acid
  VARIANT 52..53: note = Xaa can be any naturally occurring amino acid
  VARIANT 55..56: note = Xaa can be any naturally occurring amino acid
  VARIANT 70: note = Xaa can be any naturally occurring amino acid
  VARIANT 72: note = Xaa can be any naturally occurring amino acid
  VARIANT 74: note = Xaa can be any naturally occurring amino acid
  VARIANT 76: note = Xaa can be any naturally occurring amino acid
  VARIANT 84..85: note = Xaa can be any naturally occurring amino acid
  VARIANT 90..91: note = Xaa can be any naturally occurring amino acid
  VARIANT 93..94: note = Xaa can be any naturally occurring amino acid
  VARIANT 97: note = Xaa can be any naturally occurring amino acid
  VARIANT 100: note = Xaa can be any naturally occurring amino acid
DIQMTQSPXSLSASVGXXVTITCRXSXXIXSYLXWYQQKXGKXPXLLXYXAXXLXXGVPSRFSGSGSGTXFXLXIXSLQPEDFXXYYCQXXYXXPWXFGXGTKLEIK

SEQ ID NO: 435 - protein, 107 aa, synthetic construct
  REGION 1..107: note = Synthetic peptide
  VARIANT 25: note = Xaa can be any naturally occurring amino acid
  VARIANT 34: note = Xaa can be any naturally occurring amino acid
  VARIANT 48: note = Xaa can be any naturally occurring amino acid
  VARIANT 53: note = Xaa can be any naturally occurring amino acid
  VARIANT 55: note = Xaa can be any naturally occurring amino acid
  VARIANT 84..85: note = Xaa can be any naturally occurring amino acid
  VARIANT 90: note = Xaa can be any naturally occurring amino acid
  VARIANT 93: note = Xaa can be any naturally occurring amino acid
DIQMTQSPSSLSASVGDRVTITCRXSENIYSYLXWYQQKPGKAPKLLXYNAKXLXEGVPSRFSGSGSGTDFTLTISSLQPEDFXXYYCQXHYXVPWTFGQGTKLEIK

SEQ ID NO: 436 - protein, 117 aa, synthetic construct
  REGION 1..117: note = Synthetic peptide
  VARIANT 5: note = Xaa can be any naturally occurring amino acid
  VARIANT 11..13: note = Xaa can be any naturally occurring amino acid
  VARIANT 20: note = Xaa can be any naturally occurring amino acid
  VARIANT 29: note = Xaa can be any naturally occurring amino acid
  VARIANT 31: note = Xaa can be any naturally occurring amino acid
  VARIANT 33: note = Xaa can be any naturally occurring amino acid
  VARIANT 37..38: note = Xaa can be any naturally occurring amino acid
  VARIANT 40: note = Xaa can be any naturally occurring amino acid
  VARIANT 45: note = Xaa can be any naturally occurring amino acid
  VARIANT 48: note = Xaa can be any naturally occurring amino acid
  VARIANT 50: note = Xaa can be any naturally occurring amino acid
  VARIANT 55..57: note = Xaa can be any naturally occurring amino acid
  VARIANT 59: note = Xaa can be any naturally occurring amino acid
  VARIANT 61..62: note = Xaa can be any naturally occurring amino acid
  VARIANT 65..68: note = Xaa can be any naturally occurring amino acid
  VARIANT 70: note = Xaa can be any naturally occurring amino acid
  VARIANT 72: note = Xaa can be any naturally occurring amino acid
  VARIANT 74: note = Xaa can be any naturally occurring amino acid
  VARIANT 76: note = Xaa can be any naturally occurring amino acid
  VARIANT 79: note = Xaa can be any naturally occurring amino acid
  VARIANT 82: note = Xaa can be any naturally occurring amino acid
  VARIANT 84: note = Xaa can be any naturally occurring amino acid
  VARIANT 87: note = Xaa can be any naturally occurring amino acid
  VARIANT 91: note = Xaa can be any naturally occurring amino acid
  VARIANT 97: note = Xaa can be any naturally occurring amino acid
  VARIANT 101: note = Xaa can be any naturally occurring amino acid
  VARIANT 117: note = Xaa can be any naturally occurring amino acid
QVQLXQSGAEXXXPGASVKXSCKASGYTXTXYXMHWXXQXPGQGXEWXGXINPSXXXTXYXXKFXXXXTXTXDXSXSTXYMXLXSLXSEDXAVYYCXRGEXGGFAYWGQGTLVTVSX

SEQ ID NO: 437 - protein, 117 aa, synthetic construct
  REGION 1..117: note = Synthetic peptide
  VARIANT 20: note = Xaa can be any naturally occurring amino acid
  VARIANT 29: note = Xaa can be any naturally occurring amino acid
  VARIANT 31: note = Xaa can be any naturally occurring amino acid
  VARIANT 37: note = Xaa can be any naturally occurring amino acid
  VARIANT 45: note = Xaa can be any naturally occurring amino acid
  VARIANT 48: note = Xaa can be any naturally occurring amino acid
  VARIANT 56: note = Xaa can be any naturally occurring amino acid
  VARIANT 59: note = Xaa can be any naturally occurring amino acid
  VARIANT 61..62: note = Xaa can be any naturally occurring amino acid
  VARIANT 65..66: note = Xaa can be any naturally occurring amino acid
  VARIANT 68: note = Xaa can be any naturally occurring amino acid
  VARIANT 70: note = Xaa can be any naturally occurring amino acid
  VARIANT 74: note = Xaa can be any naturally occurring amino acid
  VARIANT 79: note = Xaa can be any naturally occurring amino acid
  VARIANT 84: note = Xaa can be any naturally occurring amino acid
  VARIANT 97: note = Xaa can be any naturally occurring amino acid
  VARIANT 101: note = Xaa can be any naturally occurring amino acid
QVQLVQSGAEVKKPGASVKXSCKASGYTXTXYTMHWXRQAPGQGXEWXGYINPSSXYTXYXXKFXXRXTXTADXSTSTXYMELXSLRSEDTAVYYCXRGEXGGFAYWGQGTLVTVSS

SEQ ID NO: 438 - protein, 112 aa, synthetic construct
  REGION 1..112: note = Synthetic peptide
  VARIANT 7: note = Xaa can be any naturally occurring amino acid
  VARIANT 14: note = Xaa can be any naturally occurring amino acid
  VARIANT 17..18: note = Xaa can be any naturally occurring amino acid
  VARIANT 31: note = Xaa can be any naturally occurring amino acid
  VARIANT 33: note = Xaa can be any naturally occurring amino acid
  VARIANT 39: note = Xaa can be any naturally occurring amino acid
  VARIANT 41..42: note = Xaa can be any naturally occurring amino acid
  VARIANT 44: note = Xaa can be any naturally occurring amino acid
  VARIANT 50..51: note = Xaa can be any naturally occurring amino acid
  VARIANT 55: note = Xaa can be any naturally occurring amino acid
  VARIANT 57: note = Xaa can be any naturally occurring amino acid
  VARIANT 81: note = Xaa can be any naturally occurring amino acid
  VARIANT 88: note = Xaa can be any naturally occurring amino acid
  VARIANT 92: note = Xaa can be any naturally occurring amino acid
  VARIANT 94..96: note = Xaa can be any naturally occurring amino acid
  VARIANT 99..100: note = Xaa can be any naturally occurring amino acid
  VARIANT 105: note = Xaa can be any naturally occurring amino acid
DVVMTQXPLSLPVXLGXXASISCRSSQSLVXSXGNTYLXWXXQXPGQSPXXNPSXTXTHYSGVPDRFSGSGSGTDFTLKIXRVEAEDXGVYXCXXXTHXXYTFGXGTKLEIK

SEQ ID NO: 439 - protein, 112 aa, synthetic construct
  REGION 1..112: note = Synthetic peptide
  VARIANT 33: note = Xaa can be any naturally occurring amino acid
  VARIANT 39: note = Xaa can be any naturally occurring amino acid
  VARIANT 55: note = Xaa can be any naturally occurring amino acid
  VARIANT 57: note = Xaa can be any naturally occurring amino acid
  VARIANT 81: note = Xaa can be any naturally occurring amino acid
  VARIANT 95..96: note = Xaa can be any naturally occurring amino acid
DVVMTQSPLSLPVTLGQPASISCRSSQSLVHSXGNTYLXWYQQRPGQSPRLLIYXVXNRFSGVPDRFSGSGSGTDFTLKIXRVEAEDVGVYYCSXXTHVPYTFGQGTKLEIK

SEQ ID NO: 440 - protein, 120 aa, synthetic construct
  REGION 1..120: note = Synthetic peptide
  VARIANT 5: note = Xaa can be any naturally occurring amino acid
  VARIANT 9: note = Xaa can be any naturally occurring amino acid
  VARIANT 11..12: note = Xaa can be any naturally occurring amino acid
  VARIANT 20: note = Xaa can be any naturally occurring amino acid
  VARIANT 38: note = Xaa can be any naturally occurring amino acid
  VARIANT 40..41: note = Xaa can be any naturally occurring amino acid
  VARIANT 43..44: note = Xaa can be any naturally occurring amino acid
  VARIANT 48: note = Xaa can be any naturally occurring amino acid
  VARIANT 61: note = Xaa can be any naturally occurring amino acid
  VARIANT 65: note = Xaa can be any naturally occurring amino acid
  VARIANT 67..68: note = Xaa can be any naturally occurring amino acid
  VARIANT 70: note = Xaa can be any naturally occurring amino acid
  VARIANT 72: note = Xaa can be any naturally occurring amino acid
  VARIANT 74: note = Xaa can be any naturally occurring amino acid
  VARIANT 76: note = Xaa can be any naturally occurring amino acid
  VARIANT 79: note = Xaa can be any naturally occurring amino acid
  VARIANT 82: note = Xaa can be any naturally occurring amino acid
  VARIANT 84: note = Xaa can be any naturally occurring amino acid
  VARIANT 87: note = Xaa can be any naturally occurring amino acid
  VARIANT 91: note = Xaa can be any naturally occurring amino acid
  VARIANT 116: note = Xaa can be any naturally occurring amino acid
QVQLXQSGXEXXKPGASVKXSCKASGYSFTGYNMNWVXQXXGXXLEWXGNIDPYYGGSTYXQKFXGXXTXTXDXSXSTXYMXLXSLXSEDXAVYYCARERSGYVFSAMDYWGQGTXVTVS

SEQ ID NO: 441 - protein, 120 aa, synthetic construct
  REGION 1..120: note = Synthetic peptide
  VARIANT 20: note = Xaa can be any naturally occurring amino acid
  VARIANT 48: note = Xaa can be any naturally occurring amino acid
  VARIANT 68: note = Xaa can be any naturally occurring amino acid
  VARIANT 70: note = Xaa can be any naturally occurring amino acid
  VARIANT 79: note = Xaa can be any naturally occurring amino acid
QVQLVQSGAEVKKPGASVKXSCKASGYSFTGYNMNWVRQAPGQGLEWXGNIDPYYGGSTYAQKFQGRXTXTRDTSTSTXYMELSSLRSEDTAVYYCARERSGYVFSAMDYWGQGTLVTVS

SEQ ID NO: 442 - protein, 112 aa, synthetic construct
  REGION 1..112: note = Synthetic peptide
  VARIANT 1: note = Xaa can be any naturally occurring amino acid
  VARIANT 3: note = Xaa can be any naturally occurring amino acid
  VARIANT 9: note = Xaa can be any naturally occurring amino acid
  VARIANT 15: note = Xaa can be any naturally occurring amino acid
  VARIANT 18..19: note = Xaa can be any naturally occurring amino acid
  VARIANT 21..22: note = Xaa can be any naturally occurring amino acid
  VARIANT 49: note = Xaa can be any naturally occurring amino acid
  VARIANT 51: note = Xaa can be any naturally occurring amino acid
  VARIANT 69: note = Xaa can be any naturally occurring amino acid
  VARIANT 78: note = Xaa can be any naturally occurring amino acid
  VARIANT 84: note = Xaa can be any naturally occurring amino acid
  VARIANT 93: note = Xaa can be any naturally occurring amino acid
  VARIANT 105: note = Xaa can be any naturally occurring amino acid
  VARIANT 111: note = Xaa can be any naturally occurring amino acid
XIXMTQSPXSLAVSXGEXXTXXCKSSQSVLFSSNQKNYLAWYQQKPGQXPXLLIYWASTRESGVPDRFXGSGSGTDFXLTISSXQAEDVAVYXCHQYLSSRTFGXGTKLEXK

SEQ ID NO: 443 - protein, 112 aa, synthetic construct
  REGION 1..112: note = Synthetic peptide
  VARIANT 19: note = Xaa can be any naturally occurring amino acid
  VARIANT 21: note = Xaa can be any naturally occurring amino acid
  VARIANT 84: note = Xaa can be any naturally occurring amino acid
DIVMTQSPDSLAVSLGERXTXNCKSSQSVLFSSNQKNYLAWYQQKPGQPPKLLIYWASTRESGVPDRFSGSGSGTDFTLTISSXQAEDVAVYYCHQYLSSRTFGQGTKLEIK

SEQ ID NO: 444 - protein, 117 aa, synthetic construct
  REGION 1..117: note = Synthetic peptide
  VARIANT 1: note = Xaa can be any naturally occurring amino acid
  VARIANT 5: note = Xaa can be any naturally occurring amino acid
  VARIANT 9: note = Xaa can be any naturally occurring amino acid
  VARIANT 11..12: note = Xaa can be any naturally occurring amino acid
  VARIANT 20: note = Xaa can be any naturally occurring amino acid
  VARIANT 38: note = Xaa can be any naturally occurring amino acid
  VARIANT 40..41: note = Xaa can be any naturally occurring amino acid
  VARIANT 43..44: note = Xaa can be any naturally occurring amino acid
  VARIANT 48: note = Xaa can be any naturally occurring amino acid
  VARIANT 61: note = Xaa can be any naturally occurring amino acid
  VARIANT 63: note = Xaa can be any naturally occurring amino acid
  VARIANT 65: note = Xaa can be any naturally occurring amino acid
  VARIANT 67: note = Xaa can be any naturally occurring amino acid
  VARIANT 69..70: note = Xaa can be any naturally occurring amino acid
  VARIANT 72..74: note = Xaa can be any naturally occurring amino acid
  VARIANT 76: note = Xaa can be any naturally occurring amino acid
  VARIANT 79: note = Xaa can be any naturally occurring amino acid
  VARIANT 84: note = Xaa can be any naturally occurring amino acid
  VARIANT 87: note = Xaa can be any naturally occurring amino acid
  VARIANT 91: note = Xaa can be any naturally occurring amino acid
  VARIANT 93: note = Xaa can be any naturally occurring amino acid
  VARIANT 112..113: note = Xaa can be any naturally occurring amino acid
XVQLXQSGXEXXKPGASVKXSCKASGYRFTDYNMHWVXQXXGXXLEWXGYINPNNGGTNYXQXFXGXVXXTXXXSXSTXYMELXSLXSEDXAXYYCARDYLYFFDCWGQGTXXTVSS

SEQ ID NO: 445 - protein, 107 aa, synthetic construct
  REGION 1..107: note = Synthetic peptide
  VARIANT 9: note = Xaa can be any naturally occurring amino acid
  VARIANT 11: note = Xaa can be any naturally occurring amino acid
  VARIANT 15: note = Xaa can be any naturally occurring amino acid
  VARIANT 17..18: note = Xaa can be any naturally occurring amino acid
  VARIANT 43: note = Xaa can be any naturally occurring amino acid
  VARIANT 45: note = Xaa can be any naturally occurring amino acid
  VARIANT 70: note = Xaa can be any naturally occurring amino acid
  VARIANT 72..74: note = Xaa can be any naturally occurring amino acid
  VARIANT 80: note = Xaa can be any naturally occurring amino acid
  VARIANT 84..85: note = Xaa can be any naturally occurring amino acid
  VARIANT 100: note = Xaa can be any naturally occurring amino acid
DIQMTQSPXSXSASXGXXVTITCLASQTIGTWLAWYQQKPGKXPXLLIYAAASLADGVPSRFSGSGSGTXFXXXISSLQXEDFXXYYCQQLYSIPRTFGXGTKLEIK

SEQ ID NO: 446 - protein, 107 aa, synthetic construct
  REGION 1..107: note = Synthetic peptide
  VARIANT 1: note = Xaa can be any naturally occurring amino acid
  VARIANT 15: note = Xaa can be any naturally occurring amino acid
  VARIANT 18..19: note = Xaa can be any naturally occurring amino acid
  VARIANT 42: note = Xaa can be any naturally occurring amino acid
  VARIANT 49: note = Xaa can be any naturally occurring amino acid
  VARIANT 63: note = Xaa can be any naturally occurring amino acid
  VARIANT 75..76: note = Xaa can be any naturally occurring amino acid
  VARIANT 78: note = Xaa can be any naturally occurring amino acid
  VARIANT 80: note = Xaa can be any naturally occurring amino acid
  VARIANT 84: note = Xaa can be any naturally occurring amino acid
  VARIANT 88: note = Xaa can be any naturally occurring amino acid
  VARIANT 93: note = Xaa can be any naturally occurring amino acid
XVQLVESGGGLVQPXGSXXLSCAASGFTFSSFGMHWVRQAPXKGLEWVXYISSDSRTIYYADXVKGRFTISRDNXXNXLXLQMXSLRXEDTAXYYCARDYGRTYEAY

SEQ ID NO: 447 - protein, 111 aa, synthetic construct
  REGION 1..111: note = Synthetic peptide
  VARIANT 4: note = Xaa can be any naturally occurring amino acid
  VARIANT 9: note = Xaa can be any naturally occurring amino acid
  VARIANT 17: note = Xaa can be any naturally occurring amino acid
  VARIANT 22: note = Xaa can be any naturally occurring amino acid
  VARIANT 64: note = Xaa can be any naturally occurring amino acid
  VARIANT 78: note = Xaa can be any naturally occurring amino acid
  VARIANT 80..84: note = Xaa can be any naturally occurring amino acid
  VARIANT 87: note = Xaa can be any naturally occurring amino acid
  VARIANT 89: note = Xaa can be any naturally occurring amino acid
  VARIANT 104: note = Xaa can be any naturally occurring amino acid
  VARIANT 110: note = Xaa can be any naturally occurring amino acid
DIVXTQSPXSLAVSLGXRATIXCRASKSVSTSGYSYIHWYQQKPGQPPKLLIYLASNLESGVPXRFSGSGSGTDFTLXIXXXXXEDXAXYYCQHSRELPLTFGXGTKLEXK

SEQ ID NO: 448 - protein, 111 aa, synthetic construct
  REGION 1..111: note = Synthetic peptide
  VARIANT 4: note = Xaa can be any naturally occurring amino acid
  VARIANT 82: note = Xaa can be any naturally occurring amino acid
  VARIANT 110: note = Xaa can be any naturally occurring amino acid
DIVXTQSPDSLAVSLGERATINCRASKSVSTSGYSYIHWYQQKPGQPPKLLIYLASNLESGVPDRFSGSGSGTDFTLTISSXQAEDVAVYYCQHSRELPLTFGQGTKLEXK

SEQ ID NO: 449 - protein, 7 aa, synthetic construct
  REGION 1..7: note = Synthetic peptide
  VARIANT 6: note = Xaa is T, V, F, or D
SSVSSXY

SEQ ID NO: 450 - skipped

SEQ ID NO: 451 - protein, 9 aa, synthetic construct
  REGION 1..9: note = Synthetic peptide
  VARIANT 2: note = Xaa is Q, H or S
  VARIANT 4: note = Xaa is H, Y, Q, or S
HXYXRSPLT

SEQ ID NO: 452 - protein, 7 aa, synthetic construct
  REGION 1..7: note = Synthetic peptide
  VARIANT 5: note = Xaa is T, V, D, or S
YTFTXYT

SEQ ID NO: 453 - protein, 9 aa, synthetic construct
  REGION 1..9: note = Synthetic peptide
  VARIANT 2: note = Xaa is N, Q, H, D, S, R, or A
AXGDYYVAY

SEQ ID NO: 454 - protein, 9 aa, synthetic construct
  REGION 1..9: note = Synthetic peptide
  VARIANT 2: note = Xaa is H, Q, S, or T
  VARIANT 5: note = Xaa is T, S, N, or G
QXHYXVPWT

SEQ ID NO: 455 - protein, 7 aa, synthetic construct
  REGION 1..7: note = Synthetic peptide
  VARIANT 5: note = Xaa is N, S, R, q, s, or a
  VARIANT 7: note = Xaa is w, h, y, or f
FTFSXYX

SEQ ID NO: 456 - protein, 10 aa, synthetic construct
  REGION 1..10: note = Synthetic peptide
  VARIANT 5: note = Xaa is S, N, A, or T
  VARIANT 7: note = Xaa is Q, S,A, or N
IRLKXDXYAT

SEQ ID NO: 457 - protein, 8 aa, synthetic construct
  REGION 1..8: note = Synthetic peptide
  VARIANT 1: note = Xaa is N, D, A, or T
  VARIANT 8: note = Xaa is S, T, A, C, Y
XDGPPSGX

SEQ ID NO: 458 - protein, 11 aa, synthetic construct
  REGION 1..11: note = Synthetic peptide
  VARIANT 7: note = Xaa is D, N, E, Q, S, or A
  VARIANT 9: note = Xaa is Q, S, A, D, or N
QSLVHSXGXTY

SEQ ID NO: 459 - skipped

SEQ ID NO: 460 - protein, 9 aa, synthetic construct
  REGION 1..9: note = Synthetic peptide
  VARIANT 2: note = Xaa is Q, H, or T
  VARIANT 3: note = Xaa is S, G, T, or D
SXXTHVPYT

SEQ ID NO: 461 - protein, 9 aa, synthetic construct
  REGION 1..9: note = Synthetic peptide
  VARIANT 3: note = Xaa is F, Y, S, or T
  VARIANT 5: note = Xaa is S, T, Y , or D
YTXTXYTMH

SEQ ID NO: 462 - protein, 8 aa, synthetic construct
  REGION 1..8: note = Synthetic peptide
  VARIANT 2: note = Xaa is Q, S, A, or N
  VARIANT 6: note = Xaa is T, S, V, D, or G
IXPSSXYT

SEQ ID NO: 463 - protein, 10 aa, synthetic construct
  REGION 1..10: note = Synthetic peptide
  VARIANT 1: note = Xaa is S, A, T, or V
  VARIANT 5: note = Xaa is L, V, or F
XRGEXGGFAY

SEQ ID NO: 464 - protein, 6 aa, synthetic construct
  REGION 1..6: note = Synthetic peptide
  VARIANT 6: note = Xaa is W, H, Y, or F
QTIGYX

SEQ ID NO: 465 - protein, 8 aa, synthetic construct
  REGION 1..8: note = Synthetic peptide
  VARIANT 3: note = Xaa is R or T
  VARIANT 8: note = Xaa is N, Q, S, or A
GYXFTDYX

SEQ ID NO: 466 - protein, 8 aa, synthetic construct
  REGION 1..8: note = Synthetic peptide
  VARIANT 2: note = Xaa is N, Q, S, or A
  VARIANT 4: note = Xaa is N, Q, S, or A
  VARIANT 5: note = Xaa is N, Q, S, or A
IXPXXGGT

SEQ ID NO: 467 - protein, 8 aa, synthetic construct
  REGION 1..8: note = Synthetic peptide
  VARIANT 8: note = Xaa is C, Y, S, or A
DYLYFFDX

SEQ ID NO: 468 - protein, 8 aa, synthetic construct
  REGION 1..8: note = Synthetic peptide
  VARIANT 4: note = Xaa is D, E, S, or A
ISSXSRTI

SEQ ID NO: 469 - protein, 9 aa, synthetic construct
  REGION 1..9: note = Synthetic peptide
DYGRTYEAY

SEQ ID NO: 470 - protein, 12 aa, synthetic construct
  REGION 1..12: note = Synthetic peptide
  VARIANT 8: note = Xaa is Q, S, A, D, or N
  VARIANT 11: note = Xaa is Q, S, A, D, or N
QSVLFSSXQKXY

SEQ ID NO: 471 - skipped

SEQ ID NO: 472 - protein, 8 aa, synthetic construct
  REGION 1..8: note = Synthetic peptide
  VARIANT 8: note = Xaa is N, Q, S, or A
GYSFTFYX

SEQ ID NO: 473 - protein, 8 aa, synthetic construct
  REGION 1..8: note = Synthetic peptide
  VARIANT 2: note = Xaa is A, S, E, or D
IXPYYGGS

SEQ ID NO: 474 - protein, 12 aa, synthetic construct
  REGION 1..12: note = Synthetic peptide
ERSGYVFSAMDY

SEQ ID NO: 475 - protein, 8 aa, synthetic construct
  REGION 1..8: note = Synthetic peptide
  VARIANT 2: note = Xaa is Q, S, A, or N
IXPSSGYT

SEQ ID NO: 476 - protein, 6 aa, synthetic construct
  REGION 1..6: note = Synthetic peptide
  VARIANT 2: note = Xaa is Q, S, A, D, or N
EXIYSY

SEQ ID NO: 477 - skipped

SEQ ID NO: 478 - protein, 116 aa, synthetic construct
  REGION 1..116: note = Synthetic peptide
  VARIANT 20: note = Xaa is M or V
  VARIANT 31: note = Xaa is T, V, D, or S
  VARIANT 48: note = Xaa is I or M
  VARIANT 52: note = Xaa is Q, S, A, or N
  VARIANT 70: note = Xaa is L or M
  VARIANT 74: note = Xaa is T or K
  VARIANT 98: note = Xaa is N, Q, H, D, S, R,or A
QVQLVQSGAEVKKPGASVKXSCKASGYTFTXYTMHWVRQAPGQGLEWXGFIXPSSGYTDYAQKFQGRVTXTADXSTSTVYMELSSLRSEDTAVYYCAXGDYYVAYWGQGTLVTVSS

SEQ ID NO: 479 - protein, 108 aa, synthetic construct
  REGION 1..108: note = Synthetic peptide
  VARIANT 4: note = Xaa is L or M
  VARIANT 21: note = Xaa is M or I
  VARIANT 32: note = Xaa is T, V, F, or D
  VARIANT 48: note = Xaa is W or L
  VARIANT 51: note = Xaa is S, t, Q or A
  VARIANT 53: note = Xaa is S,T, D or Q
  VARIANT 79: note = Xaa is M or L
  VARIANT 84: note = Xaa is A or F
  VARIANT 91: note = Xaa is Q, H or S
  VARIANT 93: note = Xaa is H, Y, Q, or S
DIQXTQSPSSLSASVGDRVTXTCTASSSVSSXYFHWYQQKPGKAPKLXIYXTXNLASGVPSRFSGSGSGTDYTLTISSXQPEDXATYYCHXYXRSPLTFGQGTKLEIK

SEQ ID NO: 480 - protein, 117 aa, synthetic construct
  REGION 1..117: note = Synthetic peptide
  VARIANT 31: note = Xaa is N, S, R, Q or A
  VARIANT 33: note = Xaa is W, H, Y or F
  VARIANT 55: note = Xaa is S, N, A, or T
  VARIANT 57: note = Xaa is Q, S, A, or N
  VARIANT 99: note = Xaa is N, D, or T
  VARIANT 106: note = Xaa is S, T, A, C, or Y
EVQLLESGGGLVQPGGSMRLSCAASGFTFSXYXMNWVRQAPGKGLEWVAQIRLKXDXYATHYADSVKGRFTISRDDSKNTVYLQMNSLRAEDTGVYYCXDGPPSGXWGQGTLLTVSS

SEQ ID NO: 481 - protein, 107 aa, synthetic construct
  REGION 1..107: note = Synthetic peptide
  VARIANT 28: note = Xaa is Q, S, A, D, or N
  VARIANT 48: note = Xaa is V or I
  VARIANT 50: note = Xaa is S, T, Q or A
  VARIANT 84: note = Xaa is G or A
  VARIANT 90: note = Xaa is H, Q, S or T
  VARIANT 93: note = Xaa is T, S, N, or G
DIQMTQSPSSLSASVGDRVTITCRISEXIYSYLAWYQQKPGKAPKLLXYXAKILVEGVPSRFSGSGSGTDFTLTISSLQPEDFXTYYCQXHYXVPWTFGQGTKLEIK

SEQ ID NO: 482 - protein, 117 aa, synthetic construct
  REGION 1..117: note = Synthetic peptide
  VARIANT 20: note = Xaa is M or V
  VARIANT 29: note = Xaa is F, Y, S or T
  VARIANT 31: note = Xaa is S, T, Y, or D
  VARIANT 37: note = Xaa is I or V
  VARIANT 45: note = Xaa is Q or L
  VARIANT 48: note = Xaa is I or M
  VARIANT 52: note = Xaa is Q, S, A, or N
  VARIANT 56: note = Xaa is T, S, V, D or G
  VARIANT 61: note = Xaa is I or A
  VARIANT 62: note = Xaa is K or Q
  VARIANT 65: note = Xaa is K or Q
  VARIANT 66: note = Xaa is D or G
  VARIANT 68: note = Xaa is A or V
  VARIANT 70: note = Xaa is L or M
  VARIANT 74: note = Xaa is T or K
  VARIANT 79: note = Xaa is A or V
  VARIANT 97: note = Xaa is S, A, T, or V
  VARIANT 101: note = Xaa is L, V, or F
QVQLVQSGAEVKKPGASVKXSCKASGYTXTXYTMHWXRQAPGQGXEWXGYIXPSSXYTHYXXKFXXRXTXTADXSTSTXYMELSSLRSEDTAVYYCXRGEXGGFAYWGQGTLVTVSS

SEQ ID NO: 483 - protein, 112 aa, synthetic construct
  REGION 1..112: note = Synthetic peptide
  VARIANT 33: note = Xaa is D, N, E, Q, S, or A
  VARIANT 35: note = Xaa is Q, S, A, D, or N
  VARIANT 55: note = Xaa is K, Q, or R
  VARIANT 57: note = Xaa is S, T, or V
  VARIANT 95: note = Xaa is Q, H, or T
  VARIANT 96: note = Xaa is S, G, T, or D
DVVMTQSPLSLPVTLGQPASISCRSSQSLVHSDGXTYLNWYQQRPGQSPRLLIYXVXNRFSGVPDRFSGSGSGTDFTLKISRVEAEDVGVYYCSXXTHVPYTFGQGTKLEIK

SEQ ID NO: 484 - protein, 121 aa, synthetic construct
  REGION 1..121: note = Synthetic peptide
  VARIANT 20: note = Xaa is I or V
  VARIANT 33: note = Xaa is N, Q, S, or A
  VARIANT 48: note = Xaa is I or M
  VARIANT 52: note = Xaa is A, S, E or D
  VARIANT 68: note = Xaa is A or V
  VARIANT 70: note = Xaa is L or M
  VARIANT 79: note = Xaa is A or V
QVQLVQSGAEVKKPGASVKXSCKASGYSFTGYXMNWVRQAPGQGLEWXGNIXPYYGGSTYAQKFQGRXTXTVDTSTSTXYMELSSLRSEDTAVYYCARERSGYVFSAMDYWGQGTLVTVSS

SEQ ID NO: 485 - protein, 112 aa, synthetic construct
  REGION 1..112: note = Synthetic peptide
  VARIANT 19: note = Xaa is V or A
  VARIANT 21: note = Xaa is M or I
  VARIANT 34: note = Xaa is Q, S, A, D, or N
  VARIANT 37: note = Xaa is Q, S, A, D, or N
  VARIANT 56: note = Xaa is H, Y, F, or W
  VARIANT 84: note = Xaa is V or L
DIVMTQSPDSLAVSLGERXTXNCKSSQSVLFSSXQKXYLAWYQQKPGQPPKLLIYXASTRESGVPDRFSGSGSGTDFTLTISSXQAEDVAVYYCHQYLSSRTFGQGTKLEIK

SEQ ID NO: 486 - protein, 118 aa, synthetic construct
  REGION 1..118: note = Synthetic peptide
  VARIANT 54: note = Xaa is D, E, S, or A
EVQLVESGGGLVQPGGSLRLSCAASGFTFSSFGMHWVRQAPGKGLEWVAYISSXSRTIYYADSVKGRFTISRDNAKNSLYLQMNSLRAEDTAVYYCARDYGRTYEAYWGQGTLVTVSS

SEQ ID NO: 487 - protein, 111 aa, synthetic construct
  REGION 1..111: note = Synthetic peptide
  VARIANT 4: note = Xaa is M or L
  VARIANT 82: note = Xaa is V or L
  VARIANT 110: note = Xaa is I or L
DIVXTQSPDSLAVSLGERATINCRASKSVSTSGYSYIHWYQQKPGQPPKLLIYLASNLESGVPDRFSGSGSGTDFTLTISSXQAEDVAVYYCQHSRELPLTFGQGTKLEIK

SEQ ID NO: 488 - protein, 117 aa, synthetic construct
  REGION 1..117: note = Synthetic peptide
  VARIANT 28: note = Xaa is R or T
  VARIANT 33: note = Xaa is N, Q, S or A
  VARIANT 52: note = Xaa is N, Q, S or A
  VARIANT 54: note = Xaa is N, Q, S or A
  VARIANT 55: note = Xaa is N, Q, S, or A
  VARIANT 106: note = Xaa is C, Y, S, or A
QVQLVQSGAEVKKPGASVKMSCKASGYXFTDYXMHWVRQAPGQGLEWIGYIXPXXGGTNYAQKFQGRATLTVDTSTSTAYMELSSLRSEDTAVYYCARDYLYFFDXWGQGTLLTVSS

SEQ ID NO: 489 - protein, 107 aa, synthetic construct
  REGION 1..107: note = Synthetic peptide
  VARIANT 24: note = Xaa is L or R
  VARIANT 32: note = Xaa is W, H, Y, or F
DIQMTQSPSSLSASVGDRVTITCLASQTIGTXLAWYQQKPGKAPKLLIYAAASLADGVPSRFSGSGSGTDFTFTISSLQPEDFVTYYCQQLYSIPRTFGQGTKLEIK

SEQ ID NO: 490 - protein, 117 aa, synthetic construct
  REGION 1..117: note = Synthetic Polypeptide
EVQLLESGGGLVQPGGSMRLSCAASGFTFSNYWMNWVRQAPGKGLEWIAQIRLKSDNYATHYTDSVKGRFTISRDDSKRTVYLQMNSLRAEDTGTYYCSDGPPSGSWGQGTLLTVSS

SEQ ID NO: 491 - protein, 117 aa, synthetic construct
  REGION 1..117: note = Synthetic Polypeptide
EVQLLESGGGLVQPGGSMRLSCAASGFTFSNYWMNWVRQAPGKGLEWIAQIRLKSDNYATHYADSVKGRFTISRDDSKRTVYLQMNSLRAEDTGTYYCSDGPPSGSWGQGTLLTVSS

SEQ ID NO: 492 - protein, 117 aa, synthetic construct
  REGION 1..117: note = Synthetic Polypeptide
EVQLLESGGGLVQPGGSMRLSCAASGFTFSNYWMNWVRQAPGKGLEWIAQIRLKSDNYVTHYTDSVKGRFTISRDDSKRTVYLQMNSLRAEDTGIYYCSDGPPSGSWGQGTLLTVSS

SEQ ID NO: 493 - protein, 117 aa, synthetic construct
  REGION 1..117: note = Synthetic Polypeptide
EVQLLESGGGLVQPGGSMRLSCAASGFTFSNYWMNWVRQAPGKGLEWIAQIRLKSDNYATHYTDSVKGRFTISRDDSKSTVYLQMNSLRAEDTGTYYCNDGPPSGSWGQGTLLTVSS

SEQ ID NO: 494 - protein, 117 aa, synthetic construct
  REGION 1..117: note = Synthetic Polypeptide
EVQLLESGGGLVQPGGSMRLSCAASGFTFSNYWMNWVRQAPGKGLEWLAQIRLKSDNYATHYTDSVKGRFTISRDDSKRTVYLQMNSLRAEDTGIYYCSDGPPSGSWGQGTLLTVSS

SEQ ID NO: 495 - protein, 117 aa, synthetic construct
  REGION 1..117: note = Synthetic Polypeptide
EVQLLESGGGLVQPGGSMRLSCAASGFTFSNYWMNWVRQAPGKGLEWLAQIRLKSDNYVTHYADSVKGRFTISRDDSKRTVYLQMNSLRAEDTGTYYCNDGPPSGSWGQGTLLTVSS

SEQ ID NO: 496 - protein, 116 aa, synthetic construct
  REGION 1..116: note = Synthetic Polypeptide
QVQLVQSGAEVKKPGASVKMSCKASGYTFTTYTIHWVRQAPGQGLEWIGFINPSSGYTEYAQKFQGRVTLTADKSTSTVYMELSSLRSEDTAVYFCANGDYYVAYWGQGTLVTVSS

SEQ ID NO: 497 - protein, 116 aa, synthetic construct
  REGION 1..116: note = Synthetic Polypeptide
QVQLVQSGAEVKKPGASVKMSCKASGYTFTTYTMHWVRQAPGQGLEWIGVINPSSGYTEYAQKFQGRVTLTADKSTSTVYMELSSLRSEDTAVYFCANGDYYVGYWGQGTLVTVSS

SEQ ID NO: 498 - protein, 116 aa, synthetic construct
  REGION 1..116: note = Synthetic Polypeptide
QVQLVQSGAEVKKPGASVKMSCKASGYTFTTYTIHWVRQAPGQGLEWIGVINPSSGYTDYAQKFQGRVTLTADKSTSTVYMELSSLRSEDTAVYFCANGDYYVGYWGQGTLVTVSS

SEQ ID NO: 499 - protein, 116 aa, synthetic construct
  REGION 1..116: note = Synthetic Polypeptide
QVQLVQSGAEVKKPGASVKMSCKASGYTFTTYTMHWVRQAPGQGLEWIGFINPSSGYTEYAQKFQGRVTLTADKSTSTVYMELSSLRSEDTAVYYCANGDYYVGYWGQGTLVTVSS

SEQ ID NO: 500 - protein, 116 aa, synthetic construct
  REGION 1..116: note = Synthetic Polypeptide
QVQLVQSGAEVKKPGASVKMSCKASGYTFTTYTIHWVRQAPGQGLEWIGVINPSSGYTEYAQKFQGRVTLTADKSTSTVYMELSSLRSEDTAVYFCANGDYYVAYWGQGTLVTVSS

SEQ ID NO: 501 - protein, 116 aa, synthetic construct
  REGION 1..116: note = Synthetic Polypeptide
QVQLVQSGAEVKKPGASVKMSCKASGYTFTTYTMHWVRQAPGQGLEWIGFINPSSGYTEYAQKFQGRVTLTADKSTSTVYMELSSLRSEDTAVYFCANGDYYVGYWGQGTLVTVSS

SEQ ID NO: 502 - protein, 117 aa, synthetic construct
  REGION 1..117: note = Synthetic Polypeptide
  VARIANT 48: note = Xaa is any aliphatic amino acid, optionally Ile or Leu
  VARIANT 59: note = Xaa is any aliphatic amino acid, optionally, Ala or Val
  VARIANT 63: note = Xaa is any aliphatic or any amino acid with a polar side chain, optionally, Ala or Thr
  VARIANT 79: note = Xaa is any basic amino acid or any amino acid with a polar side chain, optionally Arg or Ser
  VARIANT 95: note = Xaa is any aliphatic amino acid or any amino acid with a polar side chain, optionally, Thr or Ile
  VARIANT 99: note = Xaa is any acidic amino acid or any amino acid with a polar side chain, optionally, Ser or Asn
EVQLLESGGGLVQPGGSMRLSCAASGFTFSNYWMNWVRQAPGKGLEWXAQIRLKSDNYXTHYXDSVKGRFTISRDDSKXTVYLQMNSLRAEDTGXYYCXDGPPSGSWGQGTLLTVSS

SEQ ID NO: 503 - protein, 116 aa, synthetic construct
  REGION 1..116: note = Synthetic Polypeptide
  VARIANT 34: note = Xaa is any aliphatic amino acid or any amino acid comprising a sulfur containing side chain; optionally, Ile or Met
  VARIANT 50: note = Xaa is any aliphatic amino acid or any aromatic amino acid, optionally, Phe or Val
  VARIANT 59: note = Xaa is any acidic amino acid, optionally, Glu or Asp
  VARIANT 95: note = Xaa is any aromatic amino acid, optionally, Phe or Tyr
  VARIANT 104: note = Xaa is any aliphatic amino acid, optionally, Gly or Ala
QVQLVQSGAEVKKPGASVKMSCKASGYTFTTYTXHWVRQAPGQGLEWIGXINPSSGYTXYAQKFQGRVTLTADKSTSTVYMELSSLRSEDTAVYXCANGDYYVXYWGQGTLVTVSS

SEQ ID NO: 504 - protein, 8 aa, synthetic construct
  REGION 1..8: note = Synthetic Polypeptide
GYTFTTYT

SEQ ID NO: 505 - protein, 8 aa, synthetic construct
  REGION 1..8: note = Synthetic Polypeptide
INPSSGYT

SEQ ID NO: 506 - protein, 9 aa, synthetic construct
  REGION 1..9: note = Synthetic Polypeptide
  VARIANT 8: note = Xaa is any aliphatic amino acid, optionally, Gly or Ala
ANGDYYVXY

SEQ ID NO: 507 - protein, 8 aa, synthetic construct
  REGION 1..8: note = Synthetic Polypeptide
GFTFSNYW

SEQ ID NO: 508 - protein, 10 aa, synthetic construct
  REGION 1..10: note = Synthetic Polypeptide
  VARIANT 9: note = Xaa is any aliphatic amino acid, optionally, Ala or Val
IRLKSDNYXT

SEQ ID NO: 509 - protein, 8 aa, synthetic construct
  REGION 1..8: note = Synthetic Polypeptide
  VARIANT 1: note = Xaa is any acidic amino acid or any amino acid with a polar side chain, optionally, Ser or Asn
XDGPPSGS

SEQ ID NO: 510 - protein, 117 aa, synthetic construct
  REGION 1..117: note = Synthetic Polypeptide
EVKLEESGGGLVQPGGSMKLSCVASGFTFSNYWMNWVRQSPEKGLEWVAQIRLKSDNYATHYAESVKGRFTISRDDSKSSVYLQMNNLRAEDTGIYYCNDGPPSGCWGQGTTLTVSS

SEQ ID NO: 511 - protein, 107 aa, synthetic construct
  REGION 1..107: note = Synthetic Polypeptide
DIQMTQSPASLSASVGETVTITCRASENIYSYLAWYQQKQGKSPQLLVYNAKILVEGVPSRFSGSGSGTQFSLKINSLQPEDFGNYYCQHHYTVPWTFGGGTKLEIK

SEQ ID NO: 512 - protein, 108 aa, synthetic construct
  REGION 1..108: note = Synthetic Polypeptide
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPLTFGAGTKLELK

SEQ ID NO: 513 - protein, 116 aa, synthetic construct
  REGION 1..116: note = Synthetic Polypeptide
QVQLQQSAAELARPGASVKMSCKASGYTFTSYTMHWVKQRPGQGLEWIGFINPSSGYTDYNQKFKDKTTLTADKSSSTAYMQLSSLTSEDSAVYYCANGDYYVAYWGQGTLVTVSA

SEQ ID NO: 514 - protein, 10 aa, synthetic construct
  REGION 1..10: note = Synthetic
GYTFTEYTMH

SEQ ID NO: 515 - protein, 10 aa, synthetic construct
  REGION 1..10: note = Synthetic
GVNPNSGDTS

SEQ ID NO: 516 - protein, 13 aa, synthetic construct
  REGION 1..13: note = Synthetic
PGGYDVGYYAMDY

SEQ ID NO: 517 - protein, 11 aa, synthetic construct
  REGION 1..11: note = Synthetic
RASQDINNYLN

SEQ ID NO: 518 - protein, 7 aa, synthetic construct
  REGION 1..7: note = Synthetic
FTSRLHS

SEQ ID NO: 519 - protein, 9 aa, synthetic construct
  REGION 1..9: note = Synthetic
QQGYPLPWT

SEQ ID NO: 520 - protein, 10 aa, synthetic construct
  REGION 1..10: note = Synthetic
GYTFTEYTMH

SEQ ID NO: 521 - protein, 10 aa, synthetic construct
  REGION 1..10: note = Synthetic
GVNPNSGDTS

SEQ ID NO: 522 - protein, 13 aa, synthetic construct
  REGION 1..13: note = Synthetic
PGGYDVGYYAMDY

SEQ ID NO: 523 - protein, 11 aa, synthetic construct
  REGION 1..11: note = Synthetic
RASQDINNYLN

SEQ ID NO: 524 - protein, 7 aa, synthetic construct
  REGION 1..7: note = Synthetic
STSRLHS

SEQ ID NO: 525 - protein, 9 aa, synthetic construct
  REGION 1..9: note = Synthetic
QQGYPLPWT

SEQ ID NO: 526 - protein, 10 aa, synthetic construct
  REGION 1..10: note = Synthetic
GYTFTEYTMH

SEQ ID NO: 527 - protein, 10 aa, synthetic construct
  REGION 1..10: note = Synthetic
GVNPNSGDTS

SEQ ID NO: 528 - protein, 13 aa, synthetic construct
  REGION 1..13: note = Synthetic
PGGYDVGYYAMDY

SEQ ID NO: 529 - protein, 11 aa, synthetic construct
  REGION 1..11: note = Synthetic
RASQDINNYLN

SEQ ID NO: 530 - protein, 7 aa, synthetic construct
  REGION 1..7: note = Synthetic
FTSRLHS

SEQ ID NO: 531 - protein, 9 aa, synthetic construct
  REGION 1..9: note = Synthetic
QQGYPLPWT

SEQ ID NO: 532 - protein, 10 aa, synthetic construct
  REGION 1..10: note = Synthetic
GYTFTEYTMH

SEQ ID NO: 533 - protein, 10 aa, synthetic construct
  REGION 1..10: note = Synthetic
GVNPNSGDTS

SEQ ID NO: 534 - protein, 13 aa, synthetic construct
  REGION 1..13: note = Synthetic
PGGYDVGYYAMDY

SEQ ID NO: 535 - protein, 11 aa, synthetic construct
  REGION 1..11: note = Synthetic
RASQDINNYLN

SEQ ID NO: 536 - protein, 7 aa, synthetic construct
  REGION 1..7: note = Synthetic
STSRLHS

SEQ ID NO: 537 - protein, 9 aa, synthetic construct
  REGION 1..9: note = Synthetic
QQGYPLPWT